(19) United States
(12) Patent Application Publication
(10) Pub. No.: US 2024/0299370 A1
(43) Pub. Date: Sep. 12, 2024

(54) COMPOSITIONS AND FORMULATIONS FOR TOPICAL USE OF AN AKT INHIBITOR FOR THE PREVENTION, TREATMENT, AND IMPROVEMENT OF SKIN DISEASES, CONDITIONS, AND DISORDERS

(71) Applicant: DermBiont, Inc., Boston, MA (US)

(72) Inventors: Mark DE SOUZA, Berkeley, CA (US); John KINCAID, Boston, MA (US); Karl BEUTNER, Fairfield, CA (US); Emma TAYLOR, San Francisco, CA (US); Florencia Maria GUIDALI BONJOUR, Wimbledon Chase (GB); Vanessa Alexandra COFRE URRUTIA, Clapham Common (GB); Charles Rodney GREENAWAY EVANS, Godalming (GB)

(21) Appl. No.: 18/605,099

(22) Filed: Mar. 14, 2024

Related U.S. Application Data
(63) Continuation of application No. PCT/US2022/076440, filed on Sep. 14, 2022.
(60) Provisional application No. 63/244,353, filed on Sep. 15, 2021.
(60) Provisional application No. 63/344,440, filed on May 20, 2022.
(60) Provisional application No. 63/400,014, filed on Aug. 22, 2022.

(51) Int. Cl. A61K 31/4439; A61K 9/00; A61K 47/10; A61K 47/38; A61P 17/08

(52) U.S. Cl. CPC A61K 31/4439; A61K 9/0014; A61K 47/10; A61K 47/38; A61P 17/08

(57) ABSTRACT

Provided herein is a composition including a compound having the structure:

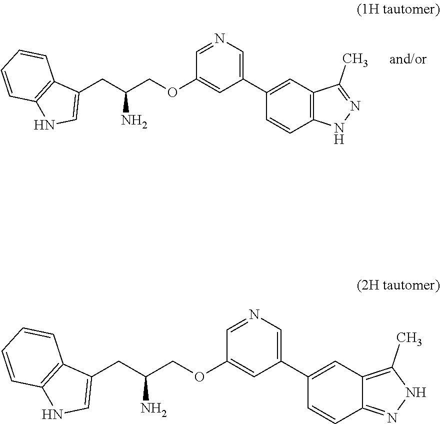 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

a pharmaceutically acceptable salt(s) thereof, and one or more excipients selected from (a)-(g): a) C₂₋₆ alcohol; b) an organic solvent and/or a penetration enhancer; c) an antioxidant; d) a preservative; e) water; f) a pH adjuster; and g) a gelling agent, wherein C₂₋₆ alcohol, the organic solvent and/or a penetration enhancer, the antioxidant, the preservative, water, the pH adjuster, and the gelling agent are defined and described herein. Methods of treating a skin disease, condition or disorder, in particular seborrheic keratosis, with the compositions are also provided herein.

### CROSS-REFERENCE

This application is a continuation of International Application No.: PCT/US2022/076440 filed on Sep. 14, 2022, which claims the benefit of U.S. Provisional application 63/244,353 filed Sep. 15, 2021, U.S. Provisional application 63/344,440 filed May 20, 2022, and U.S. Provisional application 63/400,014 filed Aug. 22, 2022; all of which are incorporated by reference herein in their entirety.

### BACKGROUND

Seborrheic Keratoses (SKs) are benign skin tumors that generally develop as an individual ages, and are most commonly found in those over the age of 50. The growths effect over 80 million Americans, are non-cancerous, but can be a nuisance or cosmetically undesirable. Typically, SKs are found on the head, neck, or torso and manifest with a number of different morphologies such as raised, flat, scaly, waxy, and light tan to black in coloring. SKs can be anywhere from a few millimeters to several centimeters in size and can be found as a single occurrence or as multiple lesions on one individual. SKs are the result of a mutation and clonal expansion of keratinocytes that proliferate in the spinous layer of the epidermis. Though they are not malignant, SKs share many characteristics with malignant tumors and more that 80% of SKs have oncogene mutations). A number of these mutations are in genes that are involved in tumor growth, keratinocyte differentiation, and apoptosis or cell death.

Despite being due to clonal mutations SKs do not have any malignant potential because unlike skin cancers SKs have normal activity of the p53 tumor suppressor gene. Akt kinase, also known as Protein Kinase B (PKB), is a central node in the tyrosine kinase/PI3K/Akt/mTOR and Ras/mitogen-activated protein kinase pathways which are tumorigenesis/tumor suppression pathways. Akt is a family of serine/threonine kinases consisting of three isoforms, named Akt1, Akt2, or Akt3. Akt1 inhibits apoptotic mechanisms and is involved in cellular survival pathways, can promote protein synthesis, and is important in cellular pathways promoting tissue growth. Increased Akt activity can block the p53 pathway preventing apoptosis or normal cell death, while simultaneously promoting unregulated growth that causes tumors. SKs show elevated levels of Akt phosphorylation which would indicate Akt is more active in these cells leading to decreased cell death and increased growth and proliferation of benign tumor forming cells.

### BRIEF SUMMARY

Current methods of treatment for SKs include superficial curettage, freezing, or shave or tangential excision, or chemical ablation of SKs using a high concentration hydrogen peroxide (40% w/w), which can cause skin irritation and damage to healthy keratinocytes. An alternative strategy for treating SKs is targeting and inhibiting Akt, and subsequently the pathway regulating tumor development in SKs.

Akt inhibitors (referred herein as Compound 1), represented by the formulas:

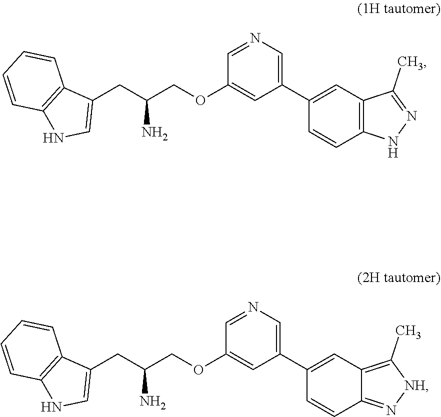 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

and
pharmaceutically acceptable salts thereof, are particularly difficult to solubilize into a composition that is easily administered, therapeutically effective, and is not irritating to the skin.

Common solvents used for formulating other small molecule compounds, such as light mineral oil or water, do not solubilize Compound 1 for use in a clinically acceptable composition. As such, there is urgent need for the development of a topical composition including Compound 1 that can be delivered topically with reduced dermal irritation to treat skin disorders such as seborrheic keratoses.

In a first aspect, the present disclosure provides a topical pharmaceutical composition. The topical pharmaceutical composition comprises a compound having the structure:

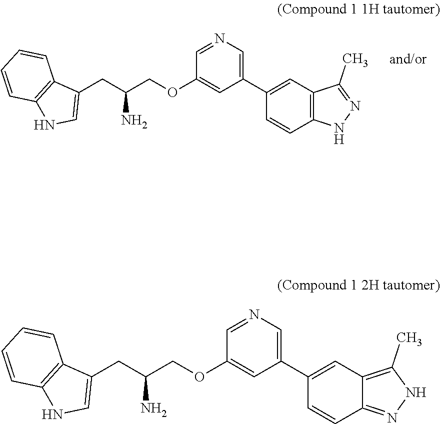 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

or a pharmaceutically acceptable salt or tautomer thereof, and one or more excipients selected from (a)-(g):
a) C₂₋₆ alcohol;
b) an organic solvent and/or a penetration enhancer;
c) an antioxidant;
d) a preservative;
e) water;
f) a pH adjuster; and
g) a gelling agent,
   wherein C₂₋₆ alcohol, the organic solvent and/or a penetration enhancer, the antioxidant, the preservative, water, the pH adjuster, and the gelling agent are defined and described herein. In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases, the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In a second aspect, the present disclosure provides a topical pharmaceutical composition, the composition comprising a compound having the structure:

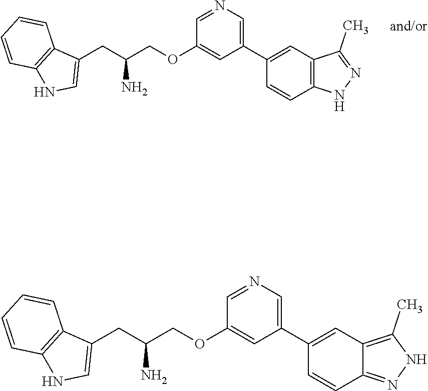 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

a pharmaceutically acceptable salt or tautomer thereof, and five or more excipients comprising ethanol, propylene glycol, 2-(2-ethoxyethoxy)ethanol, water, hydroxypropyl cellulose, optionally an antioxidant, and optionally a preservative, wherein the antioxidant and preservative are defined and described herein. In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In a third aspect, the present disclosure provides a method of treating a skin disease, condition or disorder in a subject in need thereof, the method comprising administering to the subject a composition, as described herein. In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases, the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In a fourth aspect, the present disclosure provides a kit comprising a topical pharmaceutical composition as described herein, in a tube, flexible aluminum tube or laminated plastic tube, with instructions for use.

In a further aspect, the present disclosure provides a method of treating a cutaneous lesion, the method comprising topically administering to the cutaneous lesion a composition comprising:
(i) a compound having the structure:

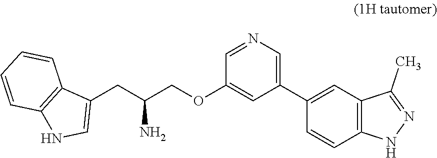 [SMILES: Cc1n[nH]c2ccc(-c3cncc(OC[C@@H](N)Cc4c[nH]c5ccccc45)c3)cc12]

or a salt thereof, and/or
(ii) a compound having the structure:

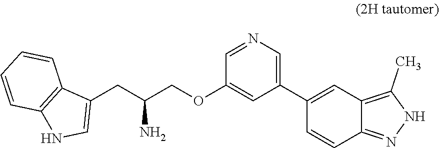 [SMILES: Cc1[nH]nc2ccc(-c3cncc(OC[C@@H](N)Cc4c[nH]c5ccccc45)c3)cc12]

or a salt thereof,
wherein the total amount of (i), (ii), or (i) and (ii) present in the composition is 0.1% to 1% by weight. Alternatively, the total amount of (i), (ii), or (i) and (ii) present in the composition is 0.01% to 10% or 0.01% to 5%.

In some embodiments, the cutaneous lesion is a keratosis. In some embodiments, the keratosis is a seborrheic keratosis. In some embodiments, the composition is administered one, two, three, or four times a day. In some embodiments, the composition is administered one, two, three, or four times a day for about 14 days to about 28 days. In some embodiments, the composition is administered one, two, three, four, or five times a week. In some embodiments, the composition is administered in a cycle comprising 1-4 daily administration for about 1-7 days, followed by no administration for about 1-7 days. In some embodiments, the cycle comprises twice daily administration for about 4 days, followed by no administration for about 4 days. In some embodiments, the cycle is repeated about 2-10 times. In some embodiments, the cycle is repeated about 7 times. In some embodiments, the composition is administered at least once daily for about 28 days (e.g., about once or twice a day). In some embodiments, the composition is administered once daily three times a week. In some embodiments, the composition is administered for a total duration of about 1, 2, 3, 4, 5, or 6 months.

In some embodiments, the method further comprises occlusion of the cutaneous lesion.

In some embodiments, the cutaneous lesion is present on a human subject. In some embodiments, the cutaneous lesion is present on the face, trunk, or an extremity, or a combination thereof, of the human subject. In some embodiments, treating comprises an improvement of one grade or more in Physician's Lesion Assessment (PLA) score as compared to before administering the composition. In some embodiments, the PLA score prior to administering is at least 2.

In some embodiments, treating comprises reducing the thickness of the cutaneous lesion to less than 1 mm. In some embodiments, the thickness of the cutaneous lesion prior administering is greater than or equal to 1 mm. In some embodiments, the length of the cutaneous lesion prior to administering is from 1 mm to 15 mm, and the width of the cutaneous lesion prior to administering is from 1 mm to 15 mm.

In some embodiments, treating comprises inducing apoptosis of a keratinocyte in the cutaneous lesion.

In some embodiments, apoptosis is measured by TUNEL assay.

In some embodiments, the composition is a gel formulation. In some embodiments, the composition comprises an alcohol, a gelling agent, or an antioxidant, or a combination of two or more thereof.

In a further aspect, the present disclosure provides a composition comprising:
(i) a compound having the structure:

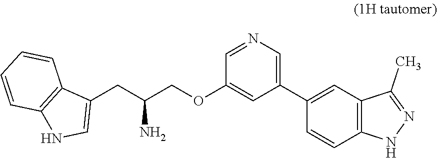 [SMILES: Cc1n[nH]c2ccc(-c3cncc(OC[C@@H](N)Cc4c[nH]c5ccccc45)c3)cc12]

or a salt thereof, and/or
(ii) a compound having the structure:

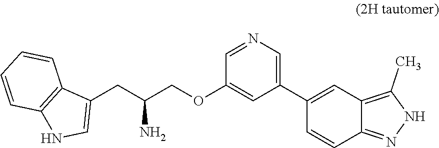 [SMILES: Cc1[nH]nc2ccc(-c3cncc(OC[C@@H](N)Cc4c[nH]c5ccccc45)c3)cc12]

or a salt thereof,
wherein the total amount of (i), (ii), or (i) and (ii) present in the composition is 0.1% to 1% by weight. Alternatively, the total amount of (i), (ii), or (i) and (ii) present in the composition is 0.01% to 10% or 0.01% to 5%.

In some embodiments, the composition is a gel formulation. In some embodiments, the composition comprises an alcohol, a gelling agent, or an antioxidant, or a combination of two or more thereof. In some embodiments, the composition further comprises a preservative. In some embodiments, the composition further comprises a gelling agent.

In a further aspect, the present disclosure provides a composition comprising:
(i) a compound having the structure:

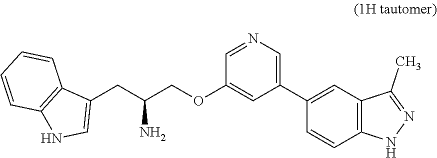 [SMILES: Cc1n[nH]c2ccc(-c3cncc(OC[C@@H](N)Cc4c[nH]c5ccccc45)c3)cc12]

or a salt thereof, and/or
(ii) a compound having the structure:

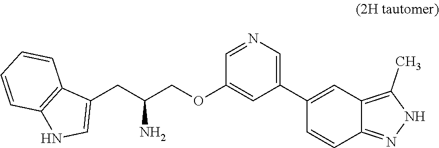 [SMILES: Cc1[nH]nc2ccc(-c3cncc(OC[C@@H](N)Cc4c[nH]c5ccccc45)c3)cc12]

or a salt thereof;
and one or more excipients selected from (a)-(e):
   a) alcohol;
   b) an organic solvent and/or a penetration enhancer;
   c) an antioxidant;
   d) a preservative; and
   e) a gelling agent.

In any of the foregoing and forthcoming aspects and embodiments, the composition may comprise an alcohol, organic solvent and/or penetration enhancer, an antioxidant, a preservative, or a gelling agent, or a combination of two or more thereof. The composition may be administered in the methods described herein.

In some embodiments, the composition comprises the alcohol. In some embodiments, the alcohol comprises ethanol, propanol, isopropanol, n-butanol, isobutanol, 2-butanol, or tert-butanol, propylene glycol, (2-(2-ethoxyethoxy)ethanol), phenoxyethanol, or a combination or two or more thereof. In some embodiments, the alcohol comprises a C₂₋₆ alcohol. In some embodiments, the C₂₋₆ alcohol comprises ethanol, propanol, isopropanol, n-butanol, isobutanol, 2-butanol, or tert-butanol, or a combination or two or more thereof. In some embodiments, the C₂₋₆ alcohol comprises ethanol. In some embodiments, the C₂₋₆ alcohol is present in an amount of 1% to 30%, from 5% to 30%, from 10% to 30%, from 5% to 20%, from 10% to 20%, or about 15% by weight of the composition. In some embodiments, the total amount of alcohol present in the composition is about 1% to about 80% by weight of the composition.

In some embodiments, the alcohol comprises an organic solvent and/or penetration enhancer.

In some embodiments, the composition comprises the organic solvent and/or penetration enhancer. In some embodiments, the organic solvent and/or penetration enhancer comprises a C₂₋₆ alkylene glycol, C₁₋₃ alkyl-(OCH₂CH₂)₁₋₅—OH, a polyethylene glycol, glycerol, a fatty alcohol, a fatty ester, or a fatty ether, or a combination of two or more thereof. In some embodiments, the C₂₋₆ alkylene glycol is propylene glycol; the C₁₋₃ alkyl-(OCH₂CH₂)₁₋₅—OH is 2-(2-ethoxyethoxy)ethanol; and the polyethylene glycol is PEG200, PEG400, or a combination thereof. In some embodiments, the organic solvent and/or penetration enhancer comprises propylene glycol and/or 2-(2-ethoxyethoxy)ethanol. In some embodiments, the organic solvent and/or penetration enhancer is present in an amount of from 50% to 99%, from 50% to 80%, from 50% to 70%, or about 60% by weight of the composition.

In some embodiments, the composition comprises the antioxidant. In some embodiments, the antioxidant comprises butylated hydroxytoluene, butylated hydroxyanisole, propyl gallate, or a combination of two or more thereof. In some embodiments, the antioxidant comprises butylated hydroxytoluene. In some embodiments, the antioxidant is present in an amount of from 0.01% to 5%, from 0.01% to 0.2%, from 0.01% to 0.10%, or about 0.05% by weight of the composition.

In some embodiments, the composition comprises the preservative. In some embodiments, the preservative comprises phenoxyethanol. In some embodiments, the preservative is present in an amount of from 0.5% to 5.0%, from 0.5% to 2%, or about 1% by weight of the composition. In some embodiments, the composition further comprises water in an amount of from 0% to 25%, from 5% to 25%, from 10% to 25%, from 15% to 25%, or about 20% by weight of the composition.

In some embodiments, the composition comprises the gelling agent. In some embodiments, the gelling agent comprises hydroxypropyl cellulose. In some embodiments, the hydroxypropyl cellulose has an average molecular weight of from 700,000 Da to 1,150,000 Da. In some embodiments, the gelling agent is present in an amount of from 0.5% to 5%, or about 2% by weight of the composition.

In some embodiments, the composition comprises ethanol, propylene glycol, 2-(2-ethoxyethoxy)ethanol, and hydroxypropyl cellulose. In some embodiments, ethanol is present in an amount of from 10% to 30%, from 10% to 20%, or about 15% by weight; propylene glycol is present in an amount of from 10% to 30%, from 10% to 20%, or about 15% by weight; 2-(2-ethoxyethoxy)ethanol is present in an amount of from 30% to 70%, from 40% to 60%, or about 47% by weight; and hydroxypropyl cellulose having an average molecular weight of from 700,000 Da to 1,150,000 Da is present in an amount of from 1% to 3% or about 2% by weight of the composition. In some embodiments, ethanol is present in an amount of about 15% by weight; propylene glycol is present in an amount of about 15% by weight; 2-(2-ethoxyethoxy)ethanol is present in an amount of about 47% by weight; and hydroxypropyl cellulose having an average molecular weight of about 850,000 Da is present in an amount of about 2% by weight of the composition.

In some embodiments, the composition further comprises an antioxidant and/or a preservative. In some embodiments, the antioxidant comprises butylated hydroxytoluene, butylated hydroxyanisole, or propyl gallate, or a combination of two or more thereof; and the preservative comprises phenoxyethanol. In some embodiments, the antioxidant comprises butylated hydroxytoluene in an amount of from 0.01% to 0.1% or about 0.05% by weight; and the preservative comprises phenoxyethanol in an amount of from 0.5% to 2%, or about 1% by weight. In some embodiments, the antioxidant comprises butylated hydroxytoluene in an amount of about 0.05% by weight; and the preservative comprises phenoxyethanol in an amount of about 1% by weight of the composition.

In some embodiments, the composition comprises a pH adjuster, optionally an acid, and further optionally a citric acid.

In some embodiments, propylene glycol is a super refined propylene glycol.

In some embodiments, 2-(2-ethoxyethoxy)ethanol is Transcutol HP having a purity of >99.90%.

In some embodiments, the total amount of (i), (ii), or (i) and (ii) present in the composition is from 0.01% to 10%, 0.1% to 10.0%, from 0.5% to 5%, from 0.5% to 2%, from 0.1% to 1%, or about 1% by weight or about 0.1% by weight of the composition.

In some embodiments, the total amount of (i), (ii), or (i) and (ii) is about 1% by weight of the composition.

In a further aspect, the present disclosure provides a composition comprising:
(i) a compound having the structure:

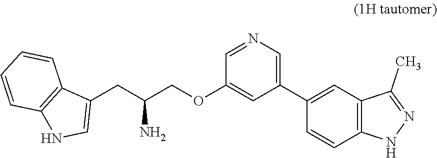 [SMILES: Cc1n[nH]c2ccc(-c3cncc(OC[C@@H](N)Cc4c[nH]c5ccccc45)c3)cc12]

or a salt thereof, and/or
(ii) a compound having the structure:

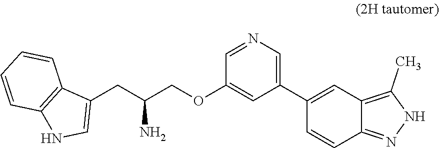 [SMILES: Cc1[nH]nc2ccc(-c3cncc(OC[C@@H](N)Cc4c[nH]c5ccccc45)c3)cc12]

or a salt thereof; and
ethanol, propylene glycol, 2-(2-ethoxyethoxy)ethanol, hydroxypropyl cellulose, and optionally an antioxidant, and optionally a preservative.

In some embodiments, the antioxidant, when present, comprises butylated hydroxytoluene; and the preservative, when present, comprises phenoxyethanol.

In some embodiments, the composition has a viscosity of about 10000 cp to about 30000 cp.

In some embodiments, the composition is a pharmaceutical composition. In some embodiments, the composition is a topical composition. In some embodiments, the composition is in the form of a gel, ointment, lotion, foam or emollient. In some embodiments, the composition is a component of a patch, tape, film, wafer, or bandage.

In one aspect, the present disclosure provides a method of treating a skin disease, condition or disorder in a subject in need thereof, comprising administering to the subject the composition described herein.

In some embodiments, the skin disease, condition or disorder is seborrheic keratosis, benign tumor, malignant tumor, parasite, virus of the skin, immune disease or disorder, or a bacterial, fungal or microbial infection. In some embodiments, the skin disease, condition or disorder is seborrheic keratosis. In some embodiments, the skin disease, condition or disorder is the benign tumor, wherein the benign tumor is a benign vascular tumor, benign fibrotic tumor, benign adipocyte tumor, benign sebaceous tumor, benign epidermal tumor, benign melanocytic lesion, or benign neural tumor; the skin disease, condition or disorder is the malignant tumor, wherein the malignant tumor is a malignant melanocytic tumor, malignant epidermal tumor, malignant vascular tumor, malignant metastatic tumor, malignant adipocyte tumor, malignant sebaceous tumor, or malignant fibrotic tumor; the skin disease, condition or disorder is the parasite, wherein the parasite is of the genus Trypanasoma or Lieshmania; the skin disease, condition or disorder is the virus, wherein the virus is molluscum contagiosum virus or human papilloma virus; or the skin disease, condition or disorder is the bacterial, fungal or microbial infection, wherein the bacterial, fungal or microbial infection is otitis media, Staphylococcus aureus infection, Mycobacterium infection, Porphyromonas infection, Salmonella infection, Chlamydia infection, tuberculosis, gingivitis or periodontal disease.

In some embodiments, the composition is applied topically to the head, scalp, face, ear(s), neck, chest, back, inframammary region(s), arm(s), leg(s), intertriginous zone(s), hand(s), foot or feet, or groin. In some embodiments, the composition is administered twice daily. In some embodiments, the composition is administered for about 2 weeks to about 8 weeks. In some embodiments, the composition is administered for about 4 weeks.

In some embodiments, the composition is administered in a pulsed cycle. In some embodiments, the composition is administered under occlusion.

In some embodiments, the skin disease, condition or disorder is skin pigmentation disorder. In some embodiments, the skin pigmentation disorder is Acanthosis nigricans.

In another aspect, the present disclosure provides a kit comprising a topical pharmaceutical composition in a tube, flexible aluminum tube or laminated plastic tube, with instructions for use.

### BRIEF DESCRIPTION OF THE DRAWINGS

FIG. 1A-FIG. 1D show local skin tolerability of four (4) compositions of Example 2 including Compound 1 and the respective placebos following once daily or every other day dermal administration for 14 days to Gottingen Minipigs, measured by scores of Erythema and Edema.

FIG. 2 shows explant TUNEL assay images. TUNEL (brown) demonstrates increased apoptosis in the keratinocytes of human seborrheic keratosis treated with the composition of Example 5 including 1% Compound 1 as compared with untreated and vehicle treated explants. Top row: 4× magnification, Bottom row 10× magnification.

FIG. 3A. Time-to-event KM plot of when lesions have 1 point drop in PLA scores, during the first 8 weeks post treatment, stratified by cohorts. For cohort 1, median response time of 1 point PLA drop is 42 days (28 to 42 days, 95% CI). For cohort 2 and 3, median response time of 1 point PLA drop is 21 days (cohort 2 95% CI: 21 to 35 days; cohort 3 95% CI: 21 to 56 days). Censoring time is set to be the last visit.

FIG. 3B. Time-to-event KM plot of when lesions have 1 point drop in PLA scores, during the first 8 weeks post treatment, in all cohorts. For all cohorts combined, median response time of 1 point PLA drop is 21 days (21 to 35 days, 95% CI).

FIG. 3C. Time-to-event KM plot of when lesions have 1 point drop in PLA scores, during the first 8 weeks post treatment. For lesions at PLA baseline=2, median response time of 1 point PLA drop is 28 days (21 to 42 days, 95% CI). For lesions at PLA baseline=3, median response time of 1 point PLA drop is 21 days (7 to 42 days, 95% CI).

FIG. 4A. Time-to-event KM plot of when lesions have 2 points drop in PLA scores, during the first 8 weeks post treatment, stratified by cohorts. For cohort 1, median response time of 2 points PLA drop is 84 days (84 to 84 days, 95% CI). For cohort 2, median response time of 2 points PLA drop is 63 days (42 to 98 days, 95% CI). For cohort 3, median response time of 2 points PLA drop is 98 days (56 to 98 days, 95% CI). Censoring time is set to be the last visit.

FIG. 4B. Time-to-event KM plot of when lesions have 2 points drop in PLA scores, during the first 8 weeks post treatment, in all cohorts. For all cohorts combined, median response time of 2 points PLA drop is 84 days (56 to 98 days, 95% CI).

FIG. 4C. Time-to-event KM plot of when lesions have 2 points drop in PLA scores, during the first 8 weeks post treatment. For lesions at PLA baseline=2, median response time of 2 points PLA drop is 84 days (70 to 98 days, 95% CI). For lesions at PLA baseline=3, median response time of 2 points PLA drop is 56 days (28 to 56 days, 95% CI).

FIG. 5A. Time-to-event KM plot of when lesions have PLA scores drop to 0, during the 8 weeks post treatment, stratified by cohorts. For cohort 1, median response time is >84 days. For cohort 2, median response time is 70 days (56 to 98 days, 95% CI). For cohort 3, median response time is >98 days. Censoring time is set to be the last visit.

FIG. 5B. Time-to-event KM plot of when lesions have PLA scores drop to 0. In all cohorts, the overall median response time is >84 days.

FIG. 5C. Time-to-event KM plot of when lesions have PLA scores drop to 0, for lesions at PLA baseline=2, median response time is 84 days (70 to 98 days, 95% CI); for lesions at PLA baseline=3, median response time is 84 days (56 to 98 days, 95% CI).

FIG. 6. Representative photos demonstrate the response of Composition Ex. 5—Compound 1 to an SK in Cohort 1 (BID for 14 days) and an SK in a background lentigo in Cohort 2 (BID for 28 days) respectively.

FIG. 7. Time-to-event KM plot of when patients have a 1 point drop in PLA scores, during the first 28 days of treatment, stratified by cohorts (0.1% facial application, 1% facial application, and 1% truncal three times a week application).

### DETAILED DESCRIPTION OF THE INVENTION

### General

Provided herein, in various aspects, are compositions including Compound 1 (as an Akt inhibitor) or salts thereof, and methods of using these compositions for the treatment of skin diseases, conditions, or disorders. The compositions may be administered topically, thereby treating the skin diseases, conditions, or disorders. The skin diseases, conditions or disorders include any one of seborrheic keratosis, benign tumors, malignant tumors, parasites, viruses of the skin, immune diseases or disorders, and a bacterial, fungal or microbial infection. In particular, the topical compositions are useful for treating seborrheic keratosis.

### Definitions

The abbreviations used herein have their conventional meaning within the chemical and biological arts.

“Alcohol” refers to an alkyl group (e.g., C₂₋₆ alkyl), as defined within, having a hydroxy group attached to a carbon of the chain. For example, alcohols useful in the present invention include, but are not limited to, ethanol, benzyl alcohol, propanol, isopropanol, butanol, isobutanol, tert-butanol, pentanol and hexanol, among others. Alcohols useful in the present invention are fully saturated. In some embodiments, the alcohol is C₂₋₆ alcohol.

“Alkylene glycol” refers to a compound having the formula of H—[O-alkylene]-OH, wherein the alkylene group has 2 to 6, 2 to 4, or 2 to 3 carbon atoms. In some embodiments, the alkylene glycol is a C₂₋₆ alkylene glycol. In some embodiments, the C₂₋₆ alkylene glycol is propylene glycol (1,2-propanediol). In some embodiments the glycol is butylene glycol and hexylene glycol.

“Di-alkylene glycol” refers to a compound having the formula of HO-(alkylene-O)₂—H, wherein the alkylene group has 2 to 6, 2 to 4, or 2 to 3 carbon atoms. In some embodiments, the di-alkylene glycol is a di-(C₂₋₆ alkylene) glycol. In some embodiments, the di-(C₂₋₆ alkylene) glycol is dipropylene glycol. Dipropylene glycol can include one or more isomers, for example 4-oxa-2,6-heptandiol, 2-(2-hydroxy-propoxy)-propan-1-ol, 2-(2-hydroxy-1-methyl-ethoxy)-propan-1-ol, and 3,3′-oxybis(propan-1-ol).

“Polyethylene glycol” refers to a polymer having the formula of HO—(CH₂CH₂O)ₙ—OH with variations in subscript “n”. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments the PEG is methoxy PEG (macrogol monomethyl ether or polyethylene glycol monomethyl ether). In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to PEG200, PEG300, PEG400, PEG600, and PEG900. The number following the “PEG” in the name refers to the average molecular weight of the polymer.

“USP Grade” excipients refers to excipients (e.g., alcohol, propylene glycol, polyethylene glycol, such as PEG200 and PEG400, and the like) meet or exceed requirements of the United States Pharmacopeia (USP).

“Super refined” excipients refer to excipients that are stripped of their impurities. Super refining removes polar impurities (including primary and secondary oxidation products) from an excipient without altering its chemical composition. The removal of these impurities helps to reduce excipient-Active Pharmaceutical Ingredient (API) interaction and subsequent API degradation, thereby maintaining both the stability of the drug and the final composition or formulation. In addition, the removal of these impurities can minimize cellular irritation, ideal for various drug administration routes. Super Refined excipients of the present invention include a super refined propylene glycol.

“Super refined propylene glycol” or “S.R. propylene glycol” refers to a highly purified propylene glycol that can enhance drug activity and composition (or formulation) stability. In some embodiments, S.R. propylene glycol has a purity of no less than about 99.5%, 99.6%, 99.7%, 99.8%, or 99.9%. In some embodiments, S.R. propylene glycol has a purity of no less than about 99.8% or 99.9%.

“Transcutol” is represented by the formula: CH₃CH₂OCH₂CH₂OCH₂CH₂OH, which has a preferred IUPAC name of 2-(2-ethoxyethoxy)ethanol. Other names for 2-(2-Ethoxyethoxy)ethanol includes diethylene glycol monoethyl ether (abbreviated as DGME or DEGEE), diethylene glycol ethyl ether (abbreviated as DEGEE), ethyldiglycol, dioxitol, 3,6-dioxa-1-octanol, Carbitol, Carbitol Cellosolve, Polysolv DE, or Dowanal DE. Transcutol includes “Transcutol P”, “Transcutol CG”, and “Transcutol HP”.

“Transcutol P” refers to a high purity grade of 2-(2-ethoxyethoxy)ethanol. “Transcutol CG” refers to a specific grade of 2-(2-ethoxyethoxy)ethanol, which is a powerful solubilizer and efficacy booster that has been used in the cosmetic and pharmaceutical industries. “Transcutol HP” refers to a highly purified grade of 2-(2-ethoxyethoxy)ethanol that can enhance drug activity and composition (or formulation) stability. In some embodiments, Transcutol P, CG, or HP has a purity of no less than about 99.5%, 99.6%, 99.7%, 99.8%, or 99.9%. In some embodiments, Transcutol P or HP has a purity of no less than 99.8% or 99.9%. In some embodiments, Transcutol HP has a purity of about 99.90%.

“Fatty alcohol” refers to a primary alcohol with a long aliphatic chain, which is either saturated or unsaturated. The fatty alcohol can also range from as few as 4-6 carbons to as many as 22-26 carbons. The fatty alcohol includes, but is not limited to, capric alcohol, undecyl alcohol, lauryl alcohol, tridecyl alcohol, myristyl alcohol, pentadecyl alcohol, cetyl alcohol, palmitoleyl alcohol (unsaturated), heptadecyl alcohol, stearyl alcohol, oleyl alcohol (unsaturated), nonadecyl alcohol, arachidyl alcohol, heneicosyl alcohol, behenyl alcohol, erucyl alcohol (unsaturated), and lignoceryl alcohol.

“Fatty ester” or “fatty acid ester” refers to a type of ester that results from the combination of a fatty acid with an alcohol. When the alcohol is a polyethylene glycol, the fatty ester refers to a polyoxyethylene fatty ester or a polyoxyethylene fatty acid ester.

“Fatty ether” refers to a type of ether that results from the combination of a fatty alcohol with a second alcohol. When the second alcohol is a polyethylene glycol, the fatty ether refers to a polyoxyethylene fatty ether.

“Polysorbate” refers a type of fatty ester that results from an ethoxylated sorbitan (a polyethylene glycol derivative of sorbitol) with a fatty acid. Examples of polysorbates include Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate), and Polysorbate 80 (polyoxyethylene (20) sorbitan monooleate). Suitable polysorbates include, but are not limited to the Tween™ series (available from Uniqema), which includes Tween 20 (polyoxyethylene (20) sorbitan monolaurate), Tween 40 (polyoxyethylene (20) sorbitan monopalmitate), Tween 60 (polyoxyethylene (20) sorbitan monostearate), and Tween 80 (polyoxyethylene (20) sorbitan monooleate). Other suitable polysorbates include the ones listed in R. C. Rowe and P. J. Shesky, Handbook of pharmaceutical excipients, (2006), 5th ed., which is incorporated herein by reference in its entirety.

“Glyceride” refers to a fatty ester when the alcohol component is glycerol. The glyceryl fatty esters (or glycerides) produced can be monoglycerides, diglycerides, or triglycerides. “Monoglyceride” is glyceride consisting of one fatty acid chain covalently bonded to a glycerol molecule through an ester linkage. “Diglyceride” is glyceride consisting of two fatty acid chains covalently bonded to a glycerol molecule through ester linkages. “Triglyceride” is glyceride consisting of three fatty acid chains covalently bonded to a glycerol molecule through ester linkages.

“Salt” refers to acid or base salts of the compounds of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Tautomer” refers to one of two or more structural isomers which exist in equilibrium and which are readily converted from one form to another. Compound 1 can exist in 1H and/or 2H tautomer forms, as shown below:

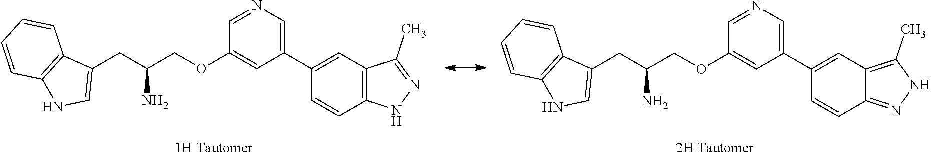 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

As used herein, “Compound 1” includes the 1H tautomer, the 2H tautomer, and mixtures of the 1H tautomer and 2H tautomer. Compound 1 also includes salts of 1H tautomer, the 2H tautomer, and mixtures of salts of the 1H tautomer and 2H tautomer.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Hydrate” refers to a compound that is complexed to water molecule. The compounds of the present invention can be complexed with 12 water molecule or from 1 to 10 water molecules.

“Composition” or “formulation” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the composition (or formulation) and not deleterious to the recipient thereof.

For any one of topical compositions as described herein, the content of water refers to a total amount by weight including the portion from a pH adjusting solution (when present) (e.g., 0.1 M, 0.5 M, or 1 M solution of citric acid in water), ethanol (if ethanol is not absolute ethanol), and the final Q.S. 100 (Q.S stands for quantum satis).

“A relative purity of the compound in the topical composition” refers to the purity of the compound (e.g., Compound 1) at a certain time point (e.g., 8 weeks) stored under stressed conditions (e.g., 40° C.) or under normal storage conditions (e.g., room temperature or 25° C.) as compared to an initial purity of the compound at time zero (i.e., day 0). As always, the relative purity of the compound at time zero (i.e., day 0) is set as 100%.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Substantially free of . . . ” refers to a composition containing no more than 1% by weight of other excipients, such as a di-(C₂₋₆ alkylene) glycol, glycerol, a fatty alcohol, a fatty ester (e.g., Polysorbate), a fatty ether, or combinations thereof, each of which is defined and described herein. Polyethylene glycol (e.g., PEG200 and/or PEG400) and/or C₁₋₃ alkyl-(OCH₂CH₂)₁₋₅—OH (e.g., 2-(2-ethoxyethoxy)ethanol or Transcutol HP) contain impurities including ethylene glycol and/or diethylene glycol. When the polyethylene glycol (e.g., PEG200 and/or PEG400) and/or C₁₋₃ alkyl-(OCH₂CH₂)₁₋₅—OH (e.g., 2-(2-ethoxyethoxy)ethanol or Transcutol HP) are present in a composition, the composition contains no more than 0.5% by weight of ethylene glycol and/or diethylene glycol as impurities. In some embodiments, when the polyethylene glycol (e.g., PEG200 and/or PEG400) and/or C₁₋₃ alkyl-(OCH₂CH₂)₁₋₅—OH (e.g., 2-(2-ethoxyethoxy)ethanol or Transcutol HP) are present in a composition, the composition contains no more than 0.25% by weight of ethylene glycol and/or diethylene glycol as impurities.

“Inhibition”, “inhibits” and “inhibitor” refer to a compound that prohibits or a method of prohibiting, a specific action or function.

“Administering” refers to providing a composition or formulation to a subject (e.g., a patient, such as a human patient) via a desired route, such as via topical administration. Topical administration may comprise, for example, application of a composition in the form of a gel, ointment, lotion, foam, emollient, or as a component of a patch, tape, film, wafer, or bandage to a surface of a subject, such as to the skin of the subject. The area over which the composition is applied may vary based upon, e.g., the condition of the subject as well as the characteristics of the composition (e.g., form, drug load, etc.).

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Patient” or “subject” refers to a living organism suffering from or prone to a disease or condition that can be treated by administration of a pharmaceutical composition as provided herein. Non-limiting examples include humans, other mammals, bovines, rats, mice, dogs, cats, primates, goat, sheep, cows, deer, and other non-mammalian animals. In some embodiments, the patient or subject is human.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Topical Compositions

As will be appreciated, some excipients of the topical compositions described herein can possess multiple functions. For example, a given substance may act as both a solvent and an enhancer, both an antioxidant and a stabilizer, both an emulsifier and a surfactant, both an emulsifier and a thickening agent, and so on. In some such cases, the function of a given substance can be considered singular, even though its properties may allow multiple functionality.

In certain aspects, the present disclosure provides a composition comprising:
(i) a compound having the structure:

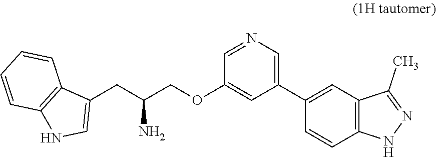 [SMILES: Cc1n[nH]c2ccc(-c3cncc(OC[C@@H](N)Cc4c[nH]c5ccccc45)c3)cc12]

or a salt thereof, and/or
(ii) a compound having the structure:

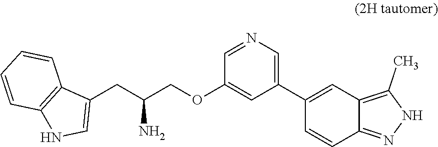 [SMILES: Cc1[nH]nc2ccc(-c3cncc(OC[C@@H](N)Cc4c[nH]c5ccccc45)c3)cc12]

or a salt thereof,
wherein the total amount of (i), (ii), or (i) and (ii) present in the composition is 0.1% to 1% by weight. Alternatively, the total amount of (i), (ii), or (i) and (ii) present in the composition is 0.01% to 10% or 0.01% to 5%.

In some embodiments, the composition is a gel formulation. In some embodiments, the composition comprises an alcohol, a gelling agent, or an antioxidant, or a combination of two or more thereof. In some embodiments, the composition further comprises a preservative. In some embodiments, the composition further comprises a gelling agent.

In certain aspects, the present disclosure provides a composition comprising:
(i) a compound having the structure:

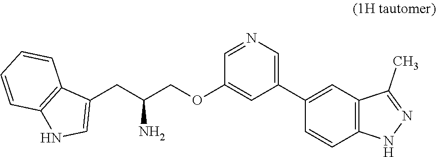 [SMILES: Cc1n[nH]c2ccc(-c3cncc(OC[C@@H](N)Cc4c[nH]c5ccccc45)c3)cc12]

or a salt thereof, and/or
(ii) a compound having the structure:

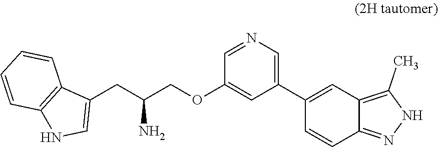 [SMILES: Cc1[nH]nc2ccc(-c3cncc(OC[C@@H](N)Cc4c[nH]c5ccccc45)c3)cc12]

or a salt thereof;
and one or more excipients selected from (a)-(e):
   a) alcohol;
   b) an organic solvent and/or a penetration enhancer;
   c) an antioxidant;
   d) a preservative; and
   e) a gelling agent.

In some embodiments, the composition comprises the alcohol. In some embodiments, the alcohol comprises ethanol, propanol, isopropanol, n-butanol, isobutanol, 2-butanol, or tert-butanol, propylene glycol, (2-(2-ethoxyethoxy)ethanol), phenoxyethanol, or a combination or two or more thereof. In some embodiments, the alcohol comprises a C₂₋₆ alcohol. In some embodiments, the C₂₋₆ alcohol comprises ethanol, propanol, isopropanol, n-butanol, isobutanol, 2-butanol, or tert-butanol, or a combination or two or more thereof. In some embodiments, the C₂₋₆ alcohol comprises ethanol. In some embodiments, the C₂₋₆ alcohol is present in an amount of 1% to 30%, from 5% to 30%, from 10% to 30%, from 5% to 20%, from 10% to 20%, or about 15% by weight of the composition.

In some embodiments, the total amount of alcohol present in the composition is about 1% to about 80% by weight of the composition.

In some embodiments, the alcohol comprises an organic solvent and/or penetration enhancer.

In some embodiments, the composition further comprises the organic solvent and/or penetration enhancer. In some embodiments, the organic solvent and/or penetration enhancer comprises a C₂₋₆ alkylene glycol, C₁₋₃ alkyl-(OCH₂CH₂)₁₋₅—OH, a polyethylene glycol, glycerol, a fatty alcohol, a fatty ester, or a fatty ether, or a combination of two or more thereof. In some embodiments, the C₂₋₆ alkylene glycol is propylene glycol; the C₁₋₃ alkyl-(OCH₂CH₂)₁₋₅—OH is 2-(2-ethoxyethoxy)ethanol; and the polyethylene glycol is PEG200, PEG400, or a combination thereof.

In some embodiments, the organic solvent and/or penetration enhancer comprises propylene glycol and/or 2-(2-ethoxyethoxy)ethanol. In some embodiments, the organic solvent and/or penetration enhancer is present in an amount of from 50% to 99%, from 50% to 80%, from 50% to 70%, or about 60% by weight of the composition.

In some embodiments, the composition further comprises the antioxidant. In some embodiments, the antioxidant comprises butylated hydroxytoluene, butylated hydroxyanisole, propyl gallate, or a combination of two or more thereof. In some embodiments, the antioxidant comprises butylated hydroxytoluene. In some embodiments, the antioxidant is present in an amount of from 0.010% to 5%, from 0.01% to 0.2%, from 0.01% to 0.1%, or about 0.05% by weight of the composition.

In some embodiments, comprising the preservative. In some embodiments, the preservative comprises phenoxyethanol. In some embodiments, the preservative is present in an amount of from 0.5% to 5.0%, from 0.5% to 2%, or about 1% by weight of the composition. In some embodiments, the composition further comprises water in an amount of from 0% to 25%, from 5% to 25%, from 10% to 25%, from 15% to 25%, or about 20% by weight of the composition.

In some embodiments, the composition comprises the gelling agent. In some embodiments, the gelling agent comprises hydroxypropyl cellulose. In some embodiments, the hydroxypropyl cellulose has an average molecular weight of from 700,000 Da to 1,150,000 Da. In some embodiments, the gelling agent is present in an amount of from 0.5% to 5%, or about 2% by weight of the composition.

In some embodiments, the composition comprises ethanol, propylene glycol, 2-(2-ethoxyethoxy)ethanol, and hydroxypropyl cellulose

In some embodiments, ethanol is present in an amount of from 10% to 30%, from 10% to 20%, or about 15% by weight; propylene glycol is present in an amount of from 10% to 30%, from 10% to 20%, or about 15% by weight; 2-(2-ethoxyethoxy)ethanol is present in an amount of from 30% to 70%, from 40% to 60%, or about 47% by weight; and hydroxypropyl cellulose having an average molecular weight of from 700,000 Da to 1,150,000 Da is present in an amount of from 1% to 3% or about 2% by weight.

In some embodiments, ethanol is present in an amount of about 15% by weight; propylene glycol is present in an amount of about 15% by weight; 2-(2-ethoxyethoxy)ethanol is present in an amount of about 47% by weight; and hydroxypropyl cellulose having an average molecular weight of about 850,000 Da is present in an amount of about 2% by weight.

In some embodiments, the composition further comprises an antioxidant and/or a preservative. In some embodiments, the antioxidant comprises butylated hydroxytoluene, butylated hydroxyanisole, or propyl gallate, or a combination of two or more thereof; and the preservative comprises phenoxyethanol. In some embodiments, the antioxidant comprises butylated hydroxytoluene in an amount of from 0.01% to 0.1% or about 0.05% by weight; and the preservative comprises phenoxyethanol in an amount of from 0.5% to 2%, or about 1% by weight. In some embodiments, the antioxidant comprises butylated hydroxytoluene in an amount of about 0.05% by weight; and the preservative comprises phenoxyethanol in an amount of about 1% by weight.

In some embodiments, the composition comprises a pH adjuster, optionally an acid, and further optionally a citric acid.

In some embodiments, propylene glycol is a super refined propylene glycol.

In some embodiments, 2-(2-ethoxyethoxy)ethanol is Transcutol HP having a purity of >99.90%.

In some embodiments, the total amount of (i), (ii), or (i) and (ii) present in the composition is from 0.01% to 10%, 0.1% to 10.0%, from 0.5% to 5%, from 0.5% to 2%, from 0.1% to 1%, or about 1% by weight or about 0.1% by weight of the composition.

In some embodiments, the total amount of (i), (ii), or (i) and (ii) is about 1% by weight of the composition.

In certain aspects, the present disclosure provides a composition comprising:
(i) a compound having the structure:

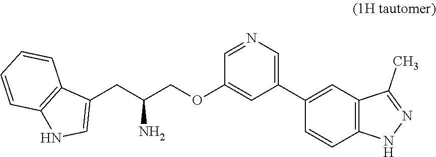 [SMILES: Cc1n[nH]c2ccc(-c3cncc(OC[C@@H](N)Cc4c[nH]c5ccccc45)c3)cc12]

or a salt thereof, and/or
(ii) a compound having the structure:

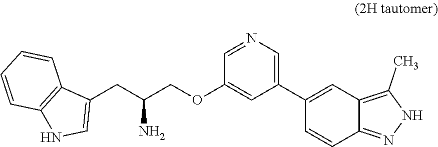 [SMILES: Cc1[nH]nc2ccc(-c3cncc(OC[C@@H](N)Cc4c[nH]c5ccccc45)c3)cc12]

or a salt thereof; and
ethanol, propylene glycol, 2-(2-ethoxyethoxy)ethanol, hydroxypropyl cellulose, and optionally an antioxidant, and optionally a preservative.

In some embodiments, the antioxidant, when present, comprises butylated hydroxytoluene; and the preservative, when present, comprises phenoxyethanol.

In some embodiments, the composition has a viscosity of about 10000 cp to about 30000 cp.

In some embodiments, the composition is a pharmaceutical composition. In some embodiments, the composition is a topical composition. In some embodiments, the composition is in the form of a gel, ointment, lotion, foam or emollient. In some embodiments, the composition is a component of a patch, tape, film, wafer, or bandage.

In certain aspects, the present disclosure provides a topical pharmaceutical composition. The topical pharmaceutical composition comprises a compound having the structure:
(i)

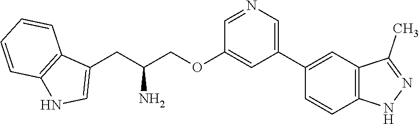 [SMILES: Cc1n[nH]c2ccc(-c3cncc(OC[C@@H](N)Cc4c[nH]c5ccccc45)c3)cc12]

and/or
(ii)

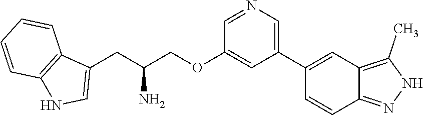 [SMILES: Cc1[nH]nc2ccc(-c3cncc(OC[C@@H](N)Cc4c[nH]c5ccccc45)c3)cc12]

or a pharmaceutically acceptable salt or tautomer thereof, and one or more excipients selected from (a)-(g):
a) C₂₋₆ alcohol;
b) an organic solvent and/or a penetration enhancer;
c) an antioxidant;
d) a preservative;
e) water;
f) a pH adjuster; and
g) a gelling agent.

In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments, the total amount of (i), (ii), or (i) and (ii) present in the composition is from 0.01% to 10%, 0.1% to 10.0%, from 0.5% to 5%, from 0.5% to 2%, from 0.1% to 1%, or about 1% by weight or about 0.1% by weight of the composition.

In some embodiments, the total amount of (i), (ii), or (i) and (ii) is about 1% by weight of the composition.

In some embodiments, the composition comprises ethanol, propylene glycol, 2-(2-ethoxyethoxy)ethanol, and hydroxypropyl cellulose.

In some embodiments, ethanol is present in an amount of from 10% to 30%, from 10% to 20%, or about 15% by weight; propylene glycol is present in an amount of from 10% to 30%, from 10% to 20%, or about 15% by weight; 2-(2-ethoxyethoxy)ethanol is present in an amount of from 30% to 70%, from 40% to 60%, or about 47% by weight; and hydroxypropyl cellulose having an average molecular weight of from 700,000 Da to 1,150,000 Da is present in an amount of from 1% to 3% or about 2% by weight.

In some embodiments, ethanol is present in an amount of about 15% by weight; propylene glycol is present in an amount of about 15% by weight; 2-(2-ethoxyethoxy)ethanol is present in an amount of about 47% by weight; and hydroxypropyl cellulose having an average molecular weight of about 850,000 Da is present in an amount of about 2% by weight.

### Excipients

### A. Alcohol

In some embodiments, a composition herein comprises an alcohol. In some embodiments, the alcohol comprises ethanol, propanol, isopropanol, n-butanol, isobutanol, 2-butanol, or tert-butanol, propylene glycol, (2-(2-ethoxyethoxy)ethanol), phenoxyethanol, or a combination or two or more thereof. In some embodiments, the alcohol comprises a C₂₋₆ alcohol. In some embodiments, the C₂₋₆ alcohol comprises ethanol, propanol, isopropanol, n-butanol, isobutanol, 2-butanol, or tert-butanol, or a combination or two or more thereof. In some embodiments, the C₂₋₆ alcohol comprises ethanol. In some embodiments, the C₂₋₆ alcohol is present in an amount of 1% to 30%, from 5% to 30%, from 10% to 30%, from 5% to 20%, from 10% to 20%, or about 15% by weight of the composition.

In some embodiments, the total amount of alcohol present in the composition is about 1% to about 80% by weight of the composition.

In some embodiments, the alcohol comprises an organic solvent and/or penetration enhancer.

In some embodiments, the alcohol comprises ethanol. In some embodiments, the alcohol comprises propylene glycol. In some embodiments, the alcohol comprises a transcutol. In some embodiments, the alcohol comprises phenoxyethanol. In some embodiments, the total amount of alcohol present in the composition is about 1% to about 80% by weight of the composition, or about 30-80%, 40-80%, 50-80%, 60-80%, 70-80%, 70%, 71%, 72%, 73%, 74%, 75%, 76%, 77%, 78%, 79%, or 80%.

### C₂-6 Alcohol

In some embodiments, the composition comprises C₂₋₆ alcohol. In some embodiments, the C₂₋₆ alcohol comprises ethanol, propanol, isopropanol, n-butanol, isobutanol, 2-butanol, benzyl alcohol, hexanol, pentanol, or tert-butanol, or combinations thereof. In some embodiments, the C₂₋₆ alcohol is ethanol or isopropanol. In some embodiments, the C₂₋₆ alcohol is ethanol. In some embodiments, the composition comprises ethanol.

In some embodiments, the composition does not include C₂₋₆ alcohol. In some embodiments, the composition does not include ethanol.

In some embodiments, the composition comprises C₂₋₆ alcohol in an amount of from 0.0% to 90%, from 5% to 40%, from 10% to 30%, from 5% to 20%, from 10% to 20%, or about 15% by weight. In some embodiments, C₂₋₆ alcohol is present in the composition in an amount of from 0.0% to 20%, from 5% to 20%, from 10% to 20%, or about 15% by weight. In some embodiments, C₂₋₆ alcohol is present in an amount of from 10% to 30%, from 10% to 20%, or about 15% by weight. In some embodiments, C₂₋₆ alcohol is present in an amount of from 10% to 20% by weight. In some embodiments, C₂₋₆ alcohol is present in an amount of about 15% by weight.

In some embodiments, the composition comprises ethanol in an amount of from 0.0% to 90%, from 5% to 40%, from 10% to 30%, from 5% to 20%, from 10% to 20%, or about 15% by weight. In some embodiments, ethanol is present in the composition in an amount of from 0.0% to 20%, from 5% to 20%, from 10% to 20%, or about 15% by weight. In some embodiments, ethanol is present in an amount of from 10% to 30%, from 10% to 20%, or about 15% by weight. In some embodiments, ethanol is present in an amount of from 10% to 20% by weight. In some embodiments, ethanol is present in an amount of about 15% by weight.

B. Organic Solvent and/or Penetration Enhancer

In some embodiments, the composition comprises an organic solvent and/or penetration enhancer. Suitable solvents and/or penetration enhancers can include a C₂₋₆ alkylene glycol (e.g., propylene glycol), a di-(C₂₋₆ alkylene) glycol (e.g., dipropylene glycol), C₁₋₃ alkyl-(OCH₂CH₂)₁₋₅—OH (e.g., 2-(2-ethoxyethoxy)ethanol or Transcutol P), a polyethylene glycol (e.g., PEG200 and/or PEG400), glycerol, a fatty alcohol (e.g., octyldodecanol), diether of an anhydrosugar alcohol (e.g. dimethyl isosorbide), a fatty ester (e.g., diisopropyl adipate, isopropyl myristate, medium-chain triglycerides, sorbitan monooleate, or the like), or a fatty ether (e.g., Laureth-4), or combinations thereof. In some embodiments, the organic solvent and/or penetration enhancer is a C₂₋₆ alkylene glycol, C₁₋₃ alkyl-(OCH₂CH₂)₁₋₅—OH, a polyethylene glycol, glycerol, a fatty alcohol, a fatty ester, or a fatty ether, or combinations thereof.

In some embodiments, the organic solvent and/or penetration enhancer is an alcohol.

In some embodiments, the organic solvent and/or penetration enhancer comprises a C₂₋₆ alkylene glycol, C₁₋₃ alkyl-(OCH₂CH₂)₁₋₅—OH, a polyethylene glycol, glycerol, a fatty alcohol, a fatty ester, or a fatty ether, or a combination of two or more thereof. In some embodiments, the C₂₋₆ alkylene glycol is propylene glycol; the C₁₋₃ alkyl-(OCH₂CH₂)₁₋₅—OH is 2-(2-ethoxyethoxy)ethanol; and the polyethylene glycol is PEG200, PEG400, or a combination thereof.

In some embodiments, the organic solvent and/or penetration enhancer comprises propylene glycol and/or 2-(2-ethoxyethoxy)ethanol. In some embodiments, the organic solvent and/or penetration enhancer is present in an amount of from 50% to 99%, from 50% to 80%, from 50% to 70%, or about 60% by weight of the composition.

In some embodiments, propylene glycol is a super refined propylene glycol.

In some embodiments, 2-(2-ethoxyethoxy)ethanol is Transcutol HP having a purity of >99.90%.

In some embodiments, the organic solvent and/or penetration enhancer is a C₂₋₆ alkylene glycol and/or C₁₋₃ alkyl-(OCH₂CH₂)₁₋₅—OH. In some embodiments, the organic solvent and/or penetration enhancer comprises a C₂₋₆ alkylene glycol. In some embodiments, the organic solvent and/or penetration enhancer comprises C₁₋₃ alkyl-(OCH₂CH₂)₁₋₅—OH. In some embodiments, the organic solvent and/or penetration enhancer comprises a C₂₋₆ alkylene glycol and C₁₋₃ alkyl-(OCH₂CH₂)₁₋₅—OH. In some embodiments, the organic solvent and/or penetration enhancer is a mixture of a C₂₋₆ alkylene glycol and C₁₋₃ alkyl-(OCH₂CH₂)₁₋₅—OH. In some embodiments, the composition comprises a C₂₋₆ alkylene glycol and C₁₋₃ alkyl-(OCH₂CH₂)₁₋₅—OH.

In some embodiments, C₁₋₃ alkyl-(OCH₂CH₂)₁₋₅—OH is 2-(2-ethoxyethoxy)ethanol (i.e., Transcutol P). In some embodiments, the C₂₋₆ alkylene glycol is propylene glycol.

In some embodiments, the organic solvent and/or penetration enhancer is propylene glycol and/or 2-(2-ethoxyethoxy)ethanol. In some embodiments, the organic solvent and/or penetration comprises propylene glycol. In some embodiments, the organic solvent and/or penetration enhancer comprises 2-(2-ethoxyethoxy)ethanol. In some embodiments, the organic solvent and/or penetration enhancer comprises propylene glycol and 2-(2-ethoxyethoxy)ethanol. In some embodiments, the organic solvent and/or penetration enhancer is a mixture of propylene glycol and 2-(2-ethoxyethoxy)ethanol. In some embodiments, the composition comprises propylene glycol and 2-(2-ethoxyethoxy)ethanol.

In some embodiments, the organic solvent and/or penetration enhancer comprises a polyethylene glycol. In some embodiments, the polyethylene glycol is PEG200, PEG300, PEG400, PEG500, PEG600, PEG700, PEG800, or PEG900, or a combination thereof. In some embodiments the organic solvent and/or penetration enhancer is methoxy PEG (macrogol monomethyl ether or polyethylene glycol monomethyl ether). In some embodiments, the polyethylene glycol is PEG-200, or PEG-400, or a combination thereof. In some embodiments, the polyethylene glycol comprises PEG200. In some embodiments, the polyethylene glycol comprises PEG400. In some embodiments, the polyethylene glycol comprises a mixture of PEG200 and PEG400. In some embodiments, the polyethylene glycol is PEG-200 and/or PEG-400. In some embodiments, the organic solvent and/or penetration enhancer comprises PEG200. In some embodiments, the organic solvent and/or penetration enhancer comprises PEG400. In some embodiments, the organic solvent and/or penetration enhancer comprises a mixture of PEG200 and PEG400. In some embodiments, the composition comprises PEG200. In some embodiments, the composition comprises PEG400. In some embodiments, the composition comprises a mixture of PEG200 and PEG400.

In some embodiments, the organic solvent and/or penetration enhancer comprises a fatty alcohol. As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12-20, 14-20, 12-18, 14-18, or 16-18 carbons on average. Suitable fatty alcohols include, but are not limited to, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, oleyl alcohol, nonadecyl alcohol, arachidyl alcohol, heneicosyl alcohol, behenyl alcohol, erucyl alcohol, lignoceryl alcohol, octyldodecanol, or mixtures thereof. In some embodiments, the organic solvent and/or penetration enhancer comprises one or more fatty alcohols selected from capric alcohol, lauryl alcohol, myristyl alcohol, cetyl alcohol, palmitoleyl alcohol, stearyl alcohol, oleyl alcohol, arachidyl alcohol, heneicosyl alcohol, behenyl alcohol, erucyl alcohol, and lignoceryl alcohol. In some embodiments, the organic solvent and/or penetration enhancer comprises octyldodecanol. In some embodiments, the composition comprises octyldodecanol.

In some embodiments, the organic solvent and/or penetration enhancer comprises a fatty ester. In some embodiments, the fatty ester is a glyceryl fatty ester, ethylene glycol monoester and diester of a fatty acid, propylene glycol monoester and diester of a fatty acid, a sorbitan ester, a C₁₋₆ alkyl ester of a fatty acid, di-(C₁₋₆ alkyl) ester of adipic acid, or combinations thereof.

In some embodiments, the fatty ester is a glyceride. In some embodiments, the glyceride is monoglycerides, diglycerides, or triglycerides. The glycerides may be optionally substituted with sulfonic acid groups, or pharmaceutically acceptable salts thereof. Suitable fatty acids for deriving glycerides of fatty acids include, but are not limited to, those described herein. In some embodiments, the glyceride is a mono-glyceride of a fatty acid having 12 to 18 carbon atoms. In some embodiments, the glyceride is a diglyceride of a fatty acid having 12 to 18 carbon atoms. In some embodiments, the glyceride is a triglyceride of a fatty acid having 12 to 18 carbon atoms (e.g., also referred herein as a medium-chain triglyceride). In some embodiments, the organic solvent and/or penetration enhancer comprises a triglyceride of a fatty acid having 12 to 18 carbon atoms (e.g., also referred herein as a medium-chain triglyceride). In some embodiments, the composition comprises a triglyceride of a fatty acid having 12 to 18 carbon atoms (e.g., also referred herein as a medium-chain triglyceride).

In some embodiments, the fatty ester is an ethylene glycol monoester of a fatty acid, a propylene glycol monoester of a fatty acid, or a C₁₋₆ alkyl ester of a fatty acid. In some embodiments, the fatty ester is an ethylene glycol monoester, a propylene glycol monoester, or a C₁₋₄ alkyl ester of a fatty acid. Suitable fatty acids for deriving any one of the ethylene glycol monoester, propylene glycol monoester, and the C₁₋₄ alkyl ester of fatty acids include, but are not limited to, those described herein. In some embodiments, the fatty ester is an ethylene glycol monoester, a propylene glycol monoester, or a C₁₋₄ alkyl ester of a fatty acid having 12 to 18 carbon atoms. Non-limiting examples of esters of a fatty acid include a laurate, a myristate, a palmitate, a stearate, or an oleate. In some embodiments, the fatty ester is isopropyl myristate. In some embodiments, the organic solvent and/or penetration enhancer comprises isopropyl myristate. In some embodiments, the composition comprises isopropyl myristate.

In some embodiments, the fatty ester is a sorbitan ester. Suitable fatty acids for deriving the sorbitan esters include, but are not limited to, those described herein. Suitable sorbitan esters include, but are not limited to, the Span™ series (available from Uniqema), which includes Span 20 (Sorbitan monolaurate), 40 (Sorbitan monopalmitate), 60 (sorbitan monostearate), 65 (sorbitan tristearate), 80 (sorbitan monooleate), and 85 (sorbitan trioleate). In some embodiments, the fatty ester is sorbitan monooleate. In some embodiments, the organic solvent and/or penetration enhancer comprises sorbitan monooleate. In some embodiments, the composition comprises sorbitan monooleate. In some embodiments the composition comprises polyoxyethylene sorbitol esters (e.g. Tween).

In some embodiments, the fatty ester is a di-(C₁₋₄ alkyl) ester of adipic acid (i.e., an adipate) or di-(C₁₋₄ alkyl) ester of sebacic acid (i.e., a sebacate). In some embodiments, the fatty ester is diisopropyl adipate. In some embodiments, the organic solvent and/or penetration enhancer comprises diisopropyl adipate. In some embodiments, the composition comprises diisopropyl adipate.

In some embodiments, the organic solvent and/or penetration enhancer comprises a fatty ether. In some embodiments, the organic solvent and/or penetration enhancer comprises a polyoxyethylene fatty ether. In some embodiments, the organic solvent and/or penetration enhancer comprises Laureth-4. In some embodiments, the composition comprises Laureth-4.

In some embodiments, the organic solvent and/or penetration enhancer is present in the composition in an amount of from 30% to 99%, from 40% to 99%, from 50% to 99%, from 50% to 80%, from 50% to 70%, or about 60% by weight. In some embodiments, the organic solvent and/or penetration enhancer is present in the composition in an amount of from 50% to 99%, from 50% to 80%, from 50% to 70%, or about 60% by weight. In some embodiments, the organic solvent and/or penetration enhancer is present in the composition in an amount of from 50% to 80%, from 50% to 70%, or about 60% by weight. In some embodiments, the organic solvent and/or penetration enhancer is present in the composition in an amount of from 50% to 70% or about 60% by weight. In some embodiments, the organic solvent and/or penetration enhancer is present in the composition in an amount of from 50% to 70% by weight. In some embodiments, the organic solvent and/or penetration enhancer is present in the composition in an amount of about 60% by weight.

In some embodiments, the organic solvent and/or penetration enhancer is propylene glycol and/or 2-(2-ethoxyethoxy)ethanol; and a total amount of which is present in the composition in an amount of from 50% to 80%, from 50% to 70%, or about 60% by weight. In some embodiments, the organic solvent and/or penetration enhancer is propylene glycol and/or 2-(2-ethoxyethoxy)ethanol; and a total amount of which is present in the composition in an amount of from 50% to 80%, from 50% to 70%, or about 60% by weight. In some embodiments, the organic solvent and/or penetration enhancer is propylene glycol and/or 2-(2-ethoxyethoxy)ethanol; and a total amount of which is present in the composition in an amount of from 50% to 70% or about 60% by weight. In some embodiments, the organic solvent and/or penetration enhancer is propylene glycol and/or 2-(2-ethoxyethoxy)ethanol; and a total amount of which is present in the composition in an amount of from 50% to 70% by weight. In some embodiments, the organic solvent and/or penetration enhancer is propylene glycol and/or 2-(2-ethoxyethoxy)ethanol; and a total amount of which is present in the composition in an amount of about 60% by weight.

In some embodiments, the organic solvent and/or penetration enhancer is a mixture of propylene glycol and 2-(2-ethoxyethoxy)ethanol; and a total amount of which is present in the composition in an amount of from 50% to 80%, from 50% to 70%, or about 60% by weight. In some embodiments, the organic solvent and/or penetration enhancer is a mixture of propylene glycol and 2-(2-ethoxyethoxy)ethanol; and a total amount of which is present in the composition in an amount of from 50% to 80%, from 50% to 70%, or about 60% by weight. In some embodiments, the organic solvent and/or penetration enhancer is a mixture of propylene glycol and 2-(2-ethoxyethoxy)ethanol; and a total amount of which is present in the composition in an amount of from 50% to 70% or about 60% by weight. In some embodiments, the organic solvent and/or penetration enhancer is a mixture of propylene glycol and 2-(2-ethoxyethoxy)ethanol; and a total amount of which is present in the composition in an amount of from 50% to 70% by weight. In some embodiments, the organic solvent and/or penetration enhancer is a mixture of propylene glycol and 2-(2-ethoxyethoxy)ethanol; and a total amount of which is present in the composition in an amount of about 60% by weight.

In some embodiments, propylene glycol is present in the composition in an amount of from 10% to 30%, from 10% to 20%, or about 15% by weight. In some embodiments, propylene glycol is present in the composition in an amount of from 10% to 20% or about 15% by weight. In some embodiments, propylene glycol is present in the composition in an amount of from 10% to 20% by weight. In some embodiments, propylene glycol is present in the composition in an amount of about 15% by weight.

In some embodiments, 2-(2-ethoxyethoxy)ethanol is present in the composition in an amount of from 30% to 70%, from 40% to 60%, or about 47% by weight. In some embodiments, 2-(2-ethoxyethoxy)ethanol is present in the composition in an amount of from 40% to 60% or about 47% by weight. In some embodiments, 2-(2-ethoxyethoxy)ethanol is present in the composition in an amount of from 40% to 60% by weight. In some embodiments, 2-(2-ethoxyethoxy)ethanol is present in the composition in an amount of about 47% by weight.

In some embodiments, the composition comprises propylene glycol and 2-(2-ethoxyethoxy)ethanol; propylene glycol is present in an amount of from 10% to 20% by weight; and 2-(2-ethoxyethoxy)ethanol is present in an amount of from 40% to 60% by weight. In some embodiments, the composition comprises propylene glycol and 2-(2-ethoxyethoxy)ethanol; propylene glycol is present in an amount of about 15% by weight; and 2-(2-ethoxyethoxy)ethanol is present in an amount of about 47% by weight.

In some embodiments, propylene glycol is a super refined propylene glycol.

In some embodiments, 2-(2-ethoxyethoxy)ethanol is Transcutol HP. In some embodiments, 2-(2-ethoxyethoxy)ethanol is Transcutol HP having a purity of >99.90%.

### C. Antioxidant

In some embodiments, the composition comprises an antioxidant. In some embodiments, the composition does not include an antioxidant.

In some embodiments, the composition comprises an antioxidant; and the antioxidant is butylated hydroxytoluene, butylated hydroxyanisole, propyl gallate, alpha tocopherol, ascorbyl palmitate, ascorbic acid, citric acid or salts thereof, sodium meta/bisulfite, or combinations thereof. In some embodiments, the antioxidant is butylated hydroxytoluene. In some embodiments, the antioxidant is butylated hydroxyanisole. In some embodiments, the antioxidant is propyl gallate. In some embodiments, the antioxidant is a mixture of butylated hydroxytoluene and butylated hydroxyanisole.

In some embodiments, the antioxidant is present in the composition in an amount of from 0.001% to 5% by weight. In some embodiments, the antioxidant is present in an amount of from 0.01% to 0.5%, from 0.01% to 0.2%, from 0.01% to 0.1%, or about 0.05% by weight. In some embodiments, the antioxidant is present in an amount of from 0.01% to 0.5% by weight. In some embodiments, the antioxidant is present in an amount of from 0.01% to 0.2% by weight. In some embodiments, the antioxidant is present in an amount of from 0.01% to 0.1% by weight. In some embodiments, the antioxidant is present in an amount of about 0.05% by weight. In some embodiments, the antioxidant is present in an amount of about 0.1% by weight. In some embodiments, the antioxidant is butylated hydroxytoluene in an amount of from 0.01% to 0.1% or about 0.05% by weight. In some embodiments, the antioxidant is butylated hydroxytoluene in an amount of from 0.01% to 0.1% by weight. In some embodiments, the antioxidant is butylated hydroxytoluene in an amount of about 0.05% by weight. In some embodiments, the antioxidant is butylated hydroxyanisole in an amount of from 0.01% to 0.1% or about 0.05% by weight. In some embodiments, the antioxidant is butylated hydroxyanisole in an amount of from 0.01% to 0.1% by weight. In some embodiments, the antioxidant is butylated hydroxyanisole in an amount of about 0.05% by weight. In some embodiments, the antioxidant is propyl gallate in an amount of from 0.01% to 0.1% or about 0.05% by weight. In some embodiments, the antioxidant is propyl gallate in an amount of from 0.01% to 0.1% by weight. In some embodiments, the antioxidant is propyl gallate in an amount of about 0.05% by weight. In some embodiments, the antioxidant is a mixture of butylated hydroxytoluene and butylated hydroxyanisole, each of which is present in an amount of from 0.01% to 0.1% or about 0.05% by weight. In some embodiments, the antioxidant is a mixture of butylated hydroxytoluene and butylated hydroxyanisole, each of which is present in an amount of from 0.01% to 0.1% by weight. In some embodiments, the antioxidant is a mixture of butylated hydroxytoluene and butylated hydroxyanisole, each of which is present in an amount of about 0.05% by weight.

In some embodiments, the antioxidant comprises butylated hydroxytoluene, butylated hydroxyanisole, propyl gallate, or a combination of two or more thereof. In some embodiments, the antioxidant comprises butylated hydroxytoluene. In some embodiments, the antioxidant is present in an amount of from 0.01% to 5%, from 0.01% to 0.2%, from 0.01% to 0.1%, or about 0.05% by weight of the composition.

In some embodiments, the antioxidant, when present, comprises butylated hydroxytoluene; and the preservative, when present, comprises phenoxyethanol.

### D. Preservative

In some embodiments, the composition comprises a preservative. In some embodiments, the composition does not include a preservative.

In some embodiments, the composition comprises a preservative; and the preservative is benzyl alcohol, phenoxyethanol, methyl paraben, propyl paraben, butyl paraben, benzalkonium chloride, sodium benzoate, imidurea, chlorocresol, chloroxylenol, benzoic acid, sodium sulfite, sodium metabisulfite, boric acid, calcium acetate, or a combination thereof. In some embodiments, the preservative, when present, is benzyl alcohol. In some embodiments, the preservative, when present, is phenoxyethanol. In some embodiments, the preservative, when present, is a mixture of benzyl alcohol and phenoxyethanol.

In some embodiments, the preservative is present in the composition in an amount of from 0.01% to 5% by weight. In some embodiments, the preservative is present in an amount of from 0.5% to 5.0%, from 0.5% to 2%, or about 1% by weight. In some embodiments, the preservative is present in an amount of from 0.5% to 2% by weight. In some embodiments, the preservative is present in an amount of about 1% by weight. In some embodiments, the preservative is phenoxyethanol in an amount of from 0.5% to 5%, from 0.5% to 4%, from 0.5% to 3%, or from 0.5% to 2% by weight. In some embodiments, the preservative is phenoxyethanol in an amount of from 0.5% to 2% by weight. In some embodiments, the preservative is phenoxyethanol in an amount of about 1% by weight.

In some embodiments, the preservative comprises phenoxyethanol. In some embodiments, the preservative is present in an amount of from 0.5% to 5.0%, from 0.5% to 2%, or about 1% by weight of the composition.

In some embodiments, the composition further comprises an antioxidant and/or a preservative. In some embodiments, the antioxidant comprises butylated hydroxytoluene, butylated hydroxyanisole, or propyl gallate, or a combination of two or more thereof; and the preservative comprises phenoxyethanol. In some embodiments, the antioxidant comprises butylated hydroxytoluene in an amount of from 0.01% to 0.1% or about 0.05% by weight; and the preservative comprises phenoxyethanol in an amount of from 0.5% to 2%, or about 1% by weight. In some embodiments, the antioxidant comprises butylated hydroxytoluene in an amount of about 0.05% by weight; and the preservative comprises phenoxyethanol in an amount of about 1% by weight.

In some embodiments, the antioxidant, when present, comprises butylated hydroxytoluene; and the preservative, when present, comprises phenoxyethanol.

### E. Water

In some embodiments, the composition comprises water. In some embodiments, the composition does not include water.

In some embodiments, water is present in the composition an amount of from 0% to 80%, from 5% to 25%, from 10% to 25%, from 15% to 25%, or about 20% by weight. In some embodiments, the composition comprises water; and water is present in an amount of from 5% to 25%, from 10% to 25%, from 15% to 25%, or about 20% by weight. In some embodiments, water is present in an amount of from 10% to 25%, from 15% to 25%, or about 20% by weight. In some embodiments, water is present in an amount of from 10% to 25% by weight. In some embodiments, water is present in an amount of from 15% to 25% by weight. In some embodiments, water is present in an amount of about 20% by weight.

In some embodiments, the composition further comprises water in an amount of from 0% to 25%, from 5% to 25%, from 10% to 25%, from 15% to 25%, or about 20% by weight of the composition.

### F. pH Adjuster

In some embodiments, the composition comprises a pH adjuster. In some embodiments, the composition does not include a pH adjuster.

In some embodiments, the composition comprises a pH adjuster; and the pH adjuster is an acid.

In some embodiments, the acid is an organic acid. Suitable organic acids include, but are not limited to, citric acid, formic acid, lactic acid, benzoic acid, oxalic acid, acetic acid, and propionic acid. In some embodiments, the acid is an organic acid, such as citric acid, formic acid, lactic acid, benzoic acid, oxalic acid, acetic acid, or propionic acid. In some embodiments, the acid is an organic acid such as citric acid, formic acid, lactic acid, benzoic acid, acetic acid, or propionic acid. In some embodiments, the acid is an organic acid such as citric acid, formic acid, lactic acid, or acetic acid. In some embodiments, the acid is an organic acid such as citric acid or acetic acid. In some embodiments, the acid is an inorganic acid such as hydrochloric acid (HCl), boric acid (H₃BO₃), sulfuric acid (H₂SO₄), carbonic acid (H₂CO₃), or phosphoric acid (H₃PO₄). In some embodiments, the acid is an inorganic acid such as hydrochloric acid (HCl), boric acid (H₃BO₃), or phosphoric acid (H₃PO₄). In some embodiments, the acid is an inorganic acid such as hydrochloric acid (HCl) or phosphoric acid (H₃PO₄). In some embodiments, the acid is citric acid, acetic acid, or a combination thereof. In some embodiments, the acid is citric acid. In some embodiments, the acid is acetic acid.

The pH adjuster can be in an aqueous solution of an acid as described herein. In some embodiments, the pH adjuster is an aqueous solution of citric acid. In some embodiments, the pH adjuster is an aqueous solution of citric acid in a concentration of 0.1 M, 0.5 M, or 1 M.

### G. Gelling Agent

In some embodiments, the composition comprises a gelling agent (e.g., a polymer thickener). Gelling agents include, for example, hydrophilic and hydroalcoholic gelling agents frequently used in the cosmetic and pharmaceutical industries. In some embodiments, the gelling agent is a Carbopol (also referred to as a carbomer), carboxymethyl cellulose, ethylcellulose, gelatin, hydroxyethyl cellulose, hydroxypropyl cellulose, magnesium aluminum silicate (Veegum), methylcellulose, a poloxamer (Pluronics), polyvinyl alcohol, sodium alginate, dermacryl, acrylates, octylacrylamide copolymer, HPMC (hydroxypropyl methylcellulose), PVP (Polyvinylpyrrolidone), bentonite clay, tragacanth, xanthan gum, Sepineo P600, a polyethylene glycol having an average molecular weight of at least about 2500 Da, or a combination thereof. In some embodiments, the gelling agent comprises Sepineo P600. In some embodiments, the gelling agent is Sepineo P600. In some embodiments, the gelling agent comprises a polyethylene glycol having an average molecular weight of from about 2500 to 3500 Da (e.g., PEG3350). In some embodiments, the gelling agent is a polyethylene glycol having an average molecular weight of from about 2500 to 3500 Da (e.g., PEG3350). In some embodiments, the gelling agent comprises hydroxypropyl cellulose. In some embodiments, the gelling agent is hydroxypropyl cellulose.

In some embodiments, the hydroxypropyl cellulose has an average molecular weight of about 40,000 Dalton (Da), about 80,000 Da, about 100,000 Da, about 140,000 Da, about 180,000 Da, about 280,000 Da, about 370,000 Da, about 700,000 Da, about 850,000 Da, about 1,000,000 Da, about 1,150,000 Da, or about 2,500,000 Da. In some embodiments, the hydroxypropyl cellulose has an average molecular weight of about 140,000 Da, about 180,000 Da, about 280,000 Da, about 370,000 Da, about 700,000 Da, about 850,000 Da, about 1,000,000 Da, or about 1,150,000 Da. In some embodiments, the hydroxypropyl cellulose has an average molecular weight of about 700,000 Da, about 850,000 Da, about 1,000,000 Da, or about 1,150,000 Da. In some embodiments, the hydroxypropyl cellulose has an average molecular weight of from about 700,000 Da to about 1,150,000 Da.

Hydroxypropyl cellulose (HPC) includes, for example Nisso SSL, Nisso SL, Nisso L, Nisso LM, Nisso LMM, Nisso M, Nisso H, Nisso VH, Klucel ELF, Klucel EF, Klucel LF, Klucel JF, Klucel GF, Klucel MF, and Klucel HF.

Nisso SSL has an average molecular weight of about 40,000 Da; Nisso SL has an average molecular weight of about 100,000 Da; Nisso L has an average molecular weight of about 140,000 Da; Nisso LM has an average molecular weight of about 180,000 Da; Nisso LMM has an average molecular weight of about 280,000 Da; Nisso M has an average molecular weight of about 700,000 Da; Nisso H has an average molecular weight of about 1,000,000 Da; and Nisso VH has an average molecular weight of about 2,500,000 Da. Suitable particle sizes of Nisso HPC (i.e., Nisso SSL, Nisso SL, Nisso L, Nisso LM, Nisso LMM, Nisso M, Nisso H, and Nisso VH) in the composition include regular powder (40 mesh), fine powder (100 mesh), and super fine powder (300 mesh). See Technical date sheets of Nisso HPCs, the entirety of which is incorporated herein by reference.

In some embodiments, the hydroxypropyl cellulose is Nisso L, Nisso LM, Nisso LMM, Nisso M, or Nisso H. In some embodiments, the hydroxypropyl cellulose is Nisso LM, Nisso LMM, Nisso M, or Nisso H. In some embodiments, the hydroxypropyl cellulose is Nisso LMM, Nisso M, or Nisso H. In some embodiments, the hydroxypropyl cellulose is Nisso M or Nisso H. In some embodiments, the hydroxypropyl cellulose is Nisso M. In some embodiments, the hydroxypropyl cellulose is Nisso H.

Klucel ELF has an average molecular weight of about 40,000 Da; Klucel EF has an average molecular weight of about 80,000 Da; Klucel LF has an average molecular weight of about 95,000 Da; Klucel JF has an average molecular weight of about 140,000 Da; Klucel GF has an average molecular weight of about 370,000 Da; Klucel MF has an average molecular weight of about 850,000 Da; and Klucel HF has an average molecular weight of about 1,150,000 Da. Suitable particle sizes of Klucel HPC in the composition include regular grade and fine grade. See Technical date sheets of Klucel HPC products, the entirety of which is incorporated herein by reference.

In some embodiments, the hydroxypropyl cellulose is Klucel JF, Klucel GF, Klucel MF, or Klucel HF. In some embodiments, the hydroxypropyl cellulose is Klucel GF, Klucel MF, or Klucel HF. In some embodiments, the hydroxypropyl cellulose is Klucel GF. In some embodiments, the hydroxypropyl cellulose is Klucel MF. In some embodiments, the hydroxypropyl cellulose is Klucel HF.

When the gelling agent is present in the composition, in some embodiments, the composition has a viscosity of from 5,000 cP to 100,000 cP. When the gelling agent is present, in some embodiments, the composition has a viscosity of from 5,000 cP to 50,000 cP. When the gelling agent is present, in some embodiments, the composition has a viscosity of from 5,000 cP to 40,000 cP. When the gelling agent is present, in some embodiments, the composition has a viscosity of from 5,000 cP to 30,000 cP. When the gelling agent is present, in some embodiments, the composition has a viscosity of from 10,000 cP to 30,000 cP. When the gelling agent is present, in some embodiments, the composition has a viscosity of from 15,000 cP to 30,000 cP. When the gelling agent is present, in some embodiments, the composition has a viscosity of from 20,000 cP to 30,000 cP. In some embodiments, the composition has a viscosity of about 10000 cp to about 30000 cp.

When the hydroxypropyl cellulose is present, in some embodiments, the composition has a viscosity of from 5,000 cP to 100,000 cP. When the hydroxypropyl cellulose is present, in some embodiments, the composition has a viscosity of from 5,000 cP to 50,000 cP. When the hydroxypropyl cellulose is present, in some embodiments, the composition has a viscosity of from 5,000 cP to 40,000 cP. When the hydroxypropyl cellulose is present, in some embodiments, the composition has a viscosity of from 5,000 cP to 30,000 cP. When the hydroxypropyl cellulose is present, in some embodiments, the composition has a viscosity of from 10,000 cP to 30,000 cP. When the hydroxypropyl cellulose is present, in some embodiments, the composition has a viscosity of from 15,000 cP to 30,000 cP. When the hydroxypropyl cellulose is present, in some embodiments, the composition has a viscosity of from 20,000 cP to 30,000 cP. In some embodiments, the composition has a viscosity of about 10000 cp to about 30000 cp.

In some embodiments, the gelling agent is present in the composition in an amount of from 0.5% to 30% by weight, while the composition has a viscosity of from 5,000 cP to 100,000 cP. In some embodiments, the gelling agent is present in an amount of from 0.5% to 30% by weight, while the composition has a viscosity of from 5,000 cP to 50,000 cP. In some embodiments, the gelling agent is present in an amount of from 0.5% to 30% by weight, while the composition has a viscosity of from 5,000 cP to 40,000 cP. In some embodiments, the gelling agents are present in an amount of from 0.5% to 30% by weight, while the composition has a viscosity of from 5,000 cP to 30,000 cP. In some embodiments, the gelling agents are present in an amount of from 0.5% to 30% by weight, while the composition has a viscosity of from 10,000 cP to 30,000 cP. In some embodiments, the gelling agents are present in an amount of from 0.5% to 30% by weight, while the composition has a viscosity of from 15,000 cP to 30,000 cP. In some embodiments, the gelling agents are present in an amount of from 0.5% to 30% by weight, while the composition has a viscosity of from 20,000 cP to 30,000 cP. In some embodiments, the composition has a viscosity of about 10000 cp to about 30000 cp.

In some embodiments, the hydroxypropyl cellulose is present in an amount of from 0.5% to 5%, from 5% to 10%, from 10% to 20%, or from 20% to 30% by weight, while the composition has a viscosity of from 5,000 cP to 100,000 cP. When a hydroxypropyl cellulose having an average molecular weight of less than about 700,000 Da is used, in some embodiments, the hydroxypropyl cellulose is present in an amount of 5% to 30% by weight, while the composition has a viscosity of from 5,000 cP to 100,000 cP. In some embodiments, the hydroxypropyl cellulose is present in an amount of from 0.5% to 5%, from 0.5% to 4%, from 0.5% to about 3%, from 0.5% to 2%, from 1% to 5%, from 1% to 4%, from 1% to 3%, from 1% to 2%, or from 2% to 5% by weight while the composition has a viscosity of from 5,000 cP to 50,000 cP. In some embodiments, the hydroxypropyl cellulose is present in an amount of from 0.5% to 5%, from 0.5% to 4%, from 0.5% to about 3%, from 0.5% to 2%, from 1% to 5%, from 1% to 4%, from 1% to 3%, from 1% to 2%, or from 2% to 5% by weight while the composition has a viscosity of from 5,000 cP to 40,000 cP. In some embodiments, the hydroxypropyl cellulose is present in an amount of from 0.5% to 5%, from 0.5% to 4%, from 0.5% to about 3%, from 0.5% to 2%, from 1% to 5%, from 1% to 4%, from 1% to 3%, from 1% to 2%, or from 2% to 5% by weight while the composition has a viscosity of from 5,000 cP to 30,000 cP. In some embodiments, the hydroxypropyl cellulose is present in an amount of from 0.5% to 4%, from 0.5% to 3%, from 0.5% to 2%, from 1% to 5%, from 1% to 4%, from 1% to 3%, from 1% to 2%, or from 2% to 5% by weight, while the composition has a viscosity of from 10,000 cP to 30,000 cP. In some embodiments, the hydroxypropyl cellulose is present in an amount of from 0.5% to 4%, from 0.5% to 3%, from 0.5% to 2%, from 1% to 5%, from 1% to 4%, from 1% to 3%, from 1% to 2%, or from 2% to 5% by weight, while the composition has a viscosity of from 15,000 cP to 30,000 cP. In some embodiments, the hydroxypropyl cellulose is present in an amount of from 0.5% to 4%, from 0.5% to 3%, from 0.5% to 2%, from 1% to 5%, from 1% to 4%, from 1% to 3%, from 1% to 2%, or from 2% to 5% by weight, while the composition has a viscosity of from 20,000 cP to 30,000 cP. In some embodiments, the composition has a viscosity of about 10000 cp to about 30000 cp.

In some embodiments, the hydroxypropyl cellulose having an average molecular weight of from 700,000 Da to 1,150,000 Da is present in an amount of from 0.5% to about 2% by weight of the composition. In some embodiments, the hydroxypropyl cellulose having an average molecular weight of from 700,000 Da to 1,150,000 Da is present in an amount of from 0.5% to 2% by weight, while the composition has a viscosity of from about 5,000 cP to about 50,000 cP. In some embodiments, the hydroxypropyl cellulose having an average molecular weight of from 700,000 Da to 1,150,000 Da is present in an amount of from 0.5% to 2% by weight, while the composition has a viscosity of from about 5,000 cP to about 40,000 cP. In some embodiments, the hydroxypropyl cellulose having an average molecular weight of from 700,000 Da to 1,150,000 Da is present in an amount of from 0.5% to 2% by weight, while the composition has a viscosity of from about 5,000 cP to about 30,000 cP. In some embodiments, the hydroxypropyl cellulose having an average molecular weight of from 700,000 Da to 1,150,000 Da is present in an amount of from 0.5% to 2% by weight, while the composition has a viscosity of from 10,000 cP to 30,000 cP. In some embodiments, the hydroxypropyl cellulose having an average molecular weight of from 700,000 Da to 1,150,000 Da is present in an amount of from 0.5% to 2% by weight, while the composition has a viscosity of from 15,000 cP to 30,000 cP. In some embodiments, the hydroxypropyl cellulose having an average molecular weight of from 700,000 Da to 1,150,000 Da is present in an amount of from 0.5% to 2% by weight, while the composition has a viscosity of from 20,000 cP to 30,000 cP. In some embodiments, the composition has a viscosity of about 10000 cp to about 30000 cp.

In some embodiments, the hydroxypropyl cellulose having an average molecular weight of from 700,000 Da to 1,150,000 Da is present in an amount of about 1% by weight of the composition. In some embodiments, the hydroxypropyl cellulose having an average molecular weight of from 700,000 Da to 1,150,000 Da is present in an amount of about 2% by weight of the composition.

In some embodiments, the gelling agent comprises hydroxypropyl cellulose. In some embodiments, the hydroxypropyl cellulose has an average molecular weight of from 700,000 Da to 1,150,000 Da. In some embodiments, the gelling agent is present in an amount of from 0.5% to 5%, or about 2% by weight of the composition.

### Apparent pH Value

When the composition is a non-aqueous formulation, the pH value of the composition is an apparent pH value. When the composition includes water, the composition includes substantial amounts of other excipients (e.g., C₂₋₆ alcohol, organic solvents and/or penetration enhancers, a gelling agent). Therefore, the pH value of a partially aqueous solutions can be regarded only as an apparent pH value. According to US Pharmacopeia (USP) chapter <791>, the apparent pH value of an non-aqueous or a partially aqueous solution is anticipated for variability, which may be up to approximately 1 pH unit. See USP chapter <791>, the entirety of which is incorporated herein by reference for all purposes.

In some embodiments, when a pH adjuster is absent from a composition, the composition has an apparent pH value of from about 7.5 to about 9.5. In some embodiments, when a pH adjuster is absent from a composition, the composition has an apparent pH value of from about 7.5 to about 8.5. In some embodiments, when a pH adjuster is absent from a composition, the composition has an apparent pH value of from about 8.5 to about 9.5. In some embodiments, when a pH adjuster is absent from a composition, the composition has an apparent pH value of about 8. In some embodiments, when a pH adjuster is absent from a composition, the composition has an apparent pH value of about 9.

In some embodiments, when a pH adjuster is present in a composition, the composition has an apparent pH value of no more than about 7. In some embodiments, when a pH adjuster is present in a composition, the composition has an apparent pH value of from about 5 to about 7 or from about 6 to about 7. In some embodiments, when a pH adjuster is present in a composition, the composition has an apparent pH value of from about 6 to about 7.

### Compound

In any one of the compositions as described herein, the compound (i.e., Compound 1) can exist in a 1H tautomer form represented by the formula:

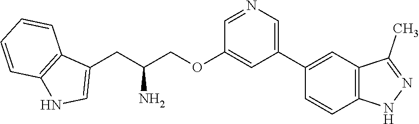 [SMILES: Cc1n[nH]c2ccc(-c3cncc(OC[C@@H](N)Cc4c[nH]c5ccccc45)c3)cc12]

or a salt thereof,
in a 2H tautomer form represented by the formula:

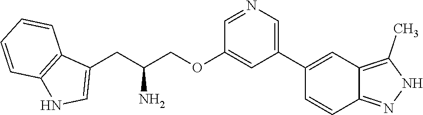 [SMILES: Cc1[nH]nc2ccc(-c3cncc(OC[C@@H](N)Cc4c[nH]c5ccccc45)c3)cc12]

or a salt thereof, or a mixture thereof.

In any one of the compositions as described herein, the compound can be a pharmaceutically acceptable salt of 1H tautomer, 2H tautomer, or a mixture thereof. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

In any one of the compositions as described herein, the compound can be in a solvate or hydrate form of 1H tautomer, 2H tautomer, or a mixture thereof.

In some embodiments, the compound is 1H tautomer or a pharmaceutically acceptable salt thereof.

In some embodiments, the compound is 2H tautomer or a pharmaceutically acceptable salt thereof.

In some embodiments, the compound is 1H tautomer, 2H tautomer, or a mixture thereof.

In some embodiments, the compound is a mixture of 1H tautomer and 2H tautomer or a pharmaceutically acceptable salt thereof. In some embodiments, the compound is a mixture of 1H tautomer and 2H tautomer or a pharmaceutically acceptable salt thereof, wherein 1H tautomer or a pharmaceutically acceptable salt thereof is present in the mixture in an amount of from 1% to 99%. In some embodiments, 1H tautomer or a pharmaceutically acceptable salt thereof is present in the mixture in an amount of from 50% to 99%, from 60% to 99%, from 70% to 99%, from 80% to 99%, or from 90% to 99%. In some embodiments, 1H tautomer or a pharmaceutically acceptable salt thereof is present in the mixture in an amount of from 80% to 99%.

In some embodiments, the compound is a mixture of 1H tautomer and 2H tautomer. In some embodiments, the compound is a mixture of 1H tautomer and 2H tautomer, wherein 1H tautomer is present in the mixture in an amount of from 1% to 99%. In some embodiments, 1H tautomer is present in the mixture in an amount of from 50% to 99%, from 60% to 99%, from 70% to 99%, from 80% to 99%, or from 90% to 99%. In some embodiments, 1H tautomer is present in the mixture in an amount of from 80% to 99%.

In some embodiments, the compound is present in the composition in an amount of from 0.01% to 10%, from 0.1% to 10.0%, from 0.5% to 5%, from 0.5% to 2%, or about 1% by weight. In some embodiments, the compound is present in an amount of from 0.5% to 5%, from 0.5% to 2%, or about 1% by weight. In some embodiments, the compound is present in an amount of from about 0.10% to about 1% by weight. In some embodiments, the compound is present in an amount of from 0.5% to 2%, or about 1% by weight. In some embodiments, the compound is present in an amount of about 1% by weight. In some embodiments, the compound is present in an amount of about 0.1% by weight.

### Embodiments

In some embodiments, the composition comprises three or more excipients from (a)-(g): a) C₂₋₆ alcohol; b) a C₂₋₆ alkylene glycol and C₁₋₃ alkyl-(OCH₂CH₂)₁₋₅—OH; c) an antioxidant; d) a preservative; e) water; f) a pH adjuster; and g) a gelling agent. In some embodiments, the composition comprises four or more excipients from (a)-(g): a) C₂₋₆ alcohol; b) a C₂₋₆ alkylene glycol and C₁₋₃ alkyl-(OCH₂CH₂)₁₋₅—OH; c) an antioxidant; d) a preservative; e) water; f) a pH adjuster; and g) a gelling agent. In some embodiments, the composition comprises five or more excipients from (a)-(g): a) C₂₋₆ alcohol; b) a C₂₋₆ alkylene glycol and C₁₋₃ alkyl-(OCH₂CH₂)₁₋₅—OH; c) an antioxidant; d) a preservative; e) water; f) a pH adjuster; and g) a gelling agent. In some embodiments, the composition comprises six or more excipients from (a)-(g): a) C₂₋₆ alcohol; b) a C₂₋₆ alkylene glycol and C₁₋₃ alkyl-(OCH₂CH₂)₁₋₅—OH; c) an antioxidant; d) a preservative; e) water; f) a pH adjuster; and g) a gelling agent. The composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments, the composition comprises a) C₂₋₆ alcohol; b) a C₂₋₆ alkylene glycol and C₁₋₃ alkyl-(OCH₂CH₂)₁₋₅—OH; e) water; and g) a gelling agent. In some embodiments, the composition comprises a) C₂₋₆ alcohol; b) a C₂₋₆ alkylene glycol and C₁₋₃ alkyl-(OCH₂CH₂)₁₋₅—OH; c) an antioxidant; d) a preservative; e) water; and g) a gelling agent. In some embodiments, the composition comprises a) C₂₋₆ alcohol; b) a C₂₋₆ alkylene glycol and C₁₋₃ alkyl-(OCH₂CH₂)₁₋₅—OH; c) an antioxidant; d) a preservative; e) water; f) a pH adjuster; and g) a gelling agent. The composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments, the present disclosure provides a topical pharmaceutical composition (A) comprising a compound having the structure:

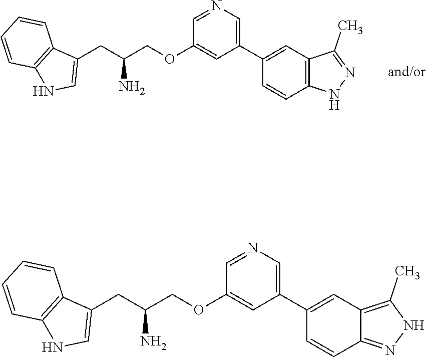 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

or a pharmaceutically acceptable salt or tautomer thereof, and four or more excipients from (a)-(g):
a) C₂₋₆ alcohol;
b) a C₂₋₆ alkylene glycol and C₁₋₃ alkyl-(OCH₂CH₂)₁₋₅—OH;
c) optionally an antioxidant;
d) optionally a preservative;
e) water;
f) optionally a pH adjuster; and
g) a gelling agent,
   wherein the compound, the C₂₋₆ alcohol, C₁₋₃ alkyl-(OCH₂CH₂)₁₋₅—OH, the antioxidant, the preservative, water, the pH adjuster, and the gelling agent are as described herein. The composition may be formulated at about 0.01% to about 10%, about 0.01% to about 5%, about 0.1% to about 10%, about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments of composition (A), the antioxidant is absent in the composition. In some embodiments of composition (A), the preservative is absent in the composition. In some embodiments of composition (A), the pH adjuster is absent in the composition. In some embodiments of composition (A), all of the antioxidant, the preservative, and the pH adjuster are absent in the composition.

In some embodiments of composition (A), one or more of the antioxidant, the preservative, and the pH adjuster are present in the composition. In some embodiments of composition (A), both the antioxidant and the preservative are present in the composition; and the pH adjuster is absent in the composition.

In some embodiments, the present disclosure provides a topical pharmaceutical composition (B) comprising a compound having the structure:

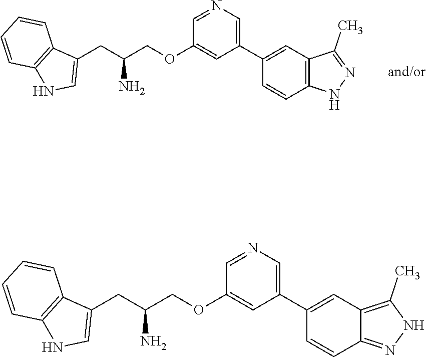 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

or a pharmaceutically acceptable salt or tautomer thereof, and excipients a), b), e), and g):
a) C₂₋₆ alcohol;
b) a C₂₋₆ alkylene glycol and C₁₋₃ alkyl-(OCH₂CH₂)₁₋₅—OH;
e) water; and
g) a gelling agent,
   wherein the compound, the C₂₋₆ alcohol, C₁₋₃ alkyl-(OCH₂CH₂)₁₋₅—OH, water, and the gelling agent are as described herein. In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments, the present disclosure provides a topical pharmaceutical composition (C) comprising a compound having the structure:

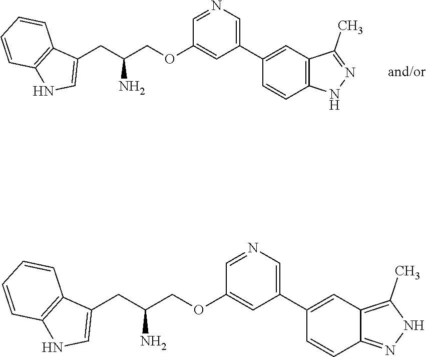 [SMILES: Cc1n[nH]c2ccc(-c3cncc(OC[C@@H](N)Cc4c[nH]c5ccccc45)c3)cc12 ; Cc1[nH]nc2ccc(-c3cncc(OC[C@@H](N)Cc4c[nH]c5ccccc45)c3)cc12]

or a pharmaceutically acceptable salt or tautomer thereof, and excipients a) to e) and g):
a) C₂₋₆ alcohol;
b) a C₂₋₆ alkylene glycol and C₁₋₃ alkyl-(OCH₂CH₂)₁₋₅—OH;
c) an antioxidant;
d) a preservative;
e) water; and
g) a gelling agent,
   wherein the compound, the C₂₋₆ alcohol, C₁₋₃ alkyl-(OCH₂CH₂)₁₋₅—OH, the antioxidant, the preservative, water, and the gelling agent are as described herein. In some embodiments, the composition may be formulated at about 0.01% to about 10%, about 0.01%, or about 10% Compound 1 (e.g., Compound 1 1H tautomer and/or Compound 1 2H tautomer) or a salt thereof. In some embodiments, the composition may be formulated at about 0.01% to about 5%, about 0.01%, or about 5% Compound 1 (e.g., Compound 1 1H tautomer and/or Compound 1 2H tautomer) or a salt thereof. In some embodiments, the composition may be formulated at about 0.1% to about 10%, about 0.1%, or about 10% Compound 1 (e.g., Compound 1 1H tautomer and/or Compound 1 2H tautomer) or a salt thereof. In some embodiments, the composition may be formulated at about 0.1% to about 1%, about 0.1%, or about 1% Compound 1 (e.g., Compound 1 1H tautomer and/or Compound 1 2H tautomer) or a salt thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks.

In some embodiments of any one of compositions (A), (B) and (C), the C₂₋₆ alcohol is present in the composition in an amount of from 10% to 30%, from 10% to 20%, or about 15% by weight. In some embodiments, the C₂₋₆ alcohol is present in an amount of from 10% to 30%. In some embodiments, the C₂₋₆ alcohol is present in an amount of from 10% to 20%. In some embodiments, the C₂₋₆ alcohol is present in an amount of about 15% by weight.

In some embodiments of any one of compositions (A), (B) and (C), the C₂₋₆ alkylene glycol is present in the composition in an amount of from 10% to 30%, from 10% to 20%, or about 15% by weight. In some embodiments, the C₂₋₆ alkylene glycol is present in an amount of from 10% to 30%. In some embodiments, the C₂₋₆ alkylene glycol is present in an amount of from 10% to 20%. In some embodiments, the C₂₋₆ alkylene glycol is present in an amount of about 15% by weight.

In some embodiments of any one of compositions (A), (B) and (C), C₁₋₃ alkyl-(OCH₂CH₂)₁₋₅—OH is present in an amount of from 30% to 70%, from 40% to 60%, or about 47% by weight. In some embodiments, C₁₋₃ alkyl-(OCH₂CH₂)₁₋₅—OH is present in an amount of from 30% to 70% by weight. In some embodiments, C₁₋₃ alkyl-(OCH₂CH₂)₁₋₅—OH is present in an amount of from 40% to 60% by weight. In some embodiments, C₁₋₃ alkyl-(OCH₂CH₂)₁₋₅—OH is present in an amount of about 47% by weight.

In some embodiments of any one of compositions (A), (B) and (C), water is present in an amount of from 10% to 30%, from 15% to 25%, or about 20% by weight. In some embodiments, water is present in an amount of from 10% to 30% by weight. In some embodiments, water is present in an amount of from 15% to 25% by weight. In some embodiments, water is present in an amount of about 20% by weight.

In some embodiments of any one of compositions (A), (B) and (C), the gelling agent is present in an amount of from 1% to 3% or about 2% by weight, while the composition has a viscosity of from 5,000 cP to 30,000 cP. In some embodiments, the gelling agent is present in an amount of from 1% to 3% or about 2% by weight, while the composition has a viscosity of from 10,000 cP to 30,000 cP. In some embodiments, the gelling agent is present in an amount of from 1% to 3% or about 2% by weight, while the composition has a viscosity of from 15,000 cP to 30,000 cP. In some embodiments, the gelling agent is present in an amount of from 1% to 3% or about 2% by weight, while the composition has a viscosity of from 20,000 cP to 30,000 cP.

In some embodiments of any one of compositions (A), (B) and (C), the C₂₋₆ alcohol is ethanol, the C₂₋₆ alkylene glycol is propylene glycol; C₁₋₃ alkyl-(OCH₂CH₂)₁₋₅—OH is 2-(2-ethoxyethoxy)ethanol; and the gelling agent is hydroxypropyl cellulose.

In some embodiments, the composition (A1) comprises the compound and four or more excipients from (a)-(g):
a) ethanol;
b) propylene glycol and 2-(2-ethoxyethoxy)ethanol;
c) optionally an antioxidant;
d) optionally a preservative;
e) water;
f) optionally a pH adjuster; and
g) hydroxypropyl cellulose.
   wherein the compound, the antioxidant, the preservative, and the pH adjuster are as described herein.

In some embodiments, the composition (B1) comprises the compound and excipients a), b), e), and g):
a) ethanol;
b) propylene glycol and 2-(2-ethoxyethoxy)ethanol;
e) water; and
g) hydroxypropyl cellulose,
   wherein the compound is as described herein.

In some embodiments, the composition (C1) comprises the compound and excipients a) to e) and g):
a) ethanol;
b) propylene glycol and 2-(2-ethoxyethoxy)ethanol;
c) an antioxidant;
d) a preservative;
e) water; and
g) hydroxypropyl cellulose,
   wherein the compound, the antioxidant and the preservative are as described herein.

In some embodiments of composition (A) or (A1), the pH adjuster, when present, is an acid. In some embodiment, the pH adjuster, when present, is citric acid.

In some embodiments of any one of compositions (A), (C), (A1), and (C1), the antioxidant, when present, is butylated hydroxytoluene, butylated hydroxyanisole, propyl gallate, or combinations thereof. In some embodiments, the antioxidant, when present, is butylated hydroxytoluene.

In some embodiments of any one of compositions (A), (C), (A1), and (C1), the preservative, when present, is phenoxyethanol.

In some embodiments of any one of compositions (A), (C), (A1), and (C1), the antioxidant, when present, is butylated hydroxytoluene, butylated hydroxyanisole, propyl gallate, or combinations thereof; and the preservative, when present, is phenoxyethanol. In some embodiments, the antioxidant, when present, is butylated hydroxytoluene; and the preservative, when present, is phenoxyethanol.

In some embodiments of any one of compositions (A), (C), (A1), and (C1), the antioxidant, when present, is present in an amount of from 0.01% to 0.1% or about 0.05% by weight. In some embodiments, the antioxidant, when present, is present in an amount of from 0.01% to 0.1% by weight. In some embodiments, the antioxidant, when present, is present in an amount of about 0.05% by weight.

In some embodiments of any one of compositions (A), (C), (A1), and (C1), the preservative, when present, is present in an amount of from 0.5% to 2% or about 1% by weight. In some embodiments, the preservative, when present, is present in an amount of from 0.5% to 2% by weight. In some embodiments, the preservative, when present, is present in an amount of about 1% by weight.

In some embodiments, the composition (C1a) comprises the compound and excipients a) to e) and g):
a) ethanol;
b) propylene glycol and 2-(2-ethoxyethoxy)ethanol;
c) butylated hydroxytoluene;
d) phenoxyethanol;
e) water; and
g) hydroxypropyl cellulose,
   wherein the compound is as described herein.

In some embodiments of any one of compositions (A1), (B1), (C1), and (C1a), ethanol is present in an amount of from 10% to 30%, from 10% to 20%, or about 15% by weight. In some embodiments, ethanol is present in an amount of from 10% to 30% by weight. In some embodiments, ethanol is present in an amount of from 10% to 20% by weight. In some embodiments, ethanol is present in an amount of about 15% by weight.

In some embodiments of any one of compositions (A1), (B1), (C1), and (C1a), propylene glycol is present in an amount of from 10% to 30%, from 10% to 20%, or about 15% by weight. In some embodiments, propylene glycol is present in an amount of from 10% to 30% by weight. In some embodiments, propylene glycol is present in an amount of from 10% to 20% by weight. In some embodiments, propylene glycol is present in an amount of about 15% by weight.

In some embodiments of any one of compositions (A1), (B1), (C1), and (C1a), 2-(2-ethoxyethoxy)ethanol is present in an amount of from 30% to 70%, from 40% to 60%, or about 47% by weight. In some embodiments, 2-(2-ethoxyethoxy)ethanol is present in an amount of from 30% to 70% by weight. In some embodiments, 2-(2-ethoxyethoxy)ethanol is present in an amount of from 40% to 60% by weight. In some embodiments, 2-(2-ethoxyethoxy)ethanol is present in an amount of about 47% by weight.

In some embodiments of any one of compositions (A1), (B1), (C1), and (C1a), water is present in an amount of from 10% to 30%, from 15% to 25%, or about 20% by weight. In some embodiments, water is present in an amount of from 10% to 30% by weight. In some embodiments, water is present in an amount of from 15% to 25% by weight. In some embodiments, water is present in an amount of about 20% by weight.

In some embodiments of any one of compositions (A1), (B1), (C1), (C1a), hydroxypropyl cellulose having an average molecular weight of from 700,000 Da to 1,150,000 Dais present in an amount of from 1% to 3% or about 2% by weight. In some embodiments, hydroxypropyl cellulose having an average molecular weight of from 700,000 Da to 1,150,000 Dais present in an amount of from 1% to 3% by weight. In some embodiments, hydroxypropyl cellulose having an average molecular weight of from 700,000 Da to 1,150,000 Da is present in an amount of about 10% by weight. In some embodiments, hydroxypropyl cellulose having an average molecular weight of from 700,000 Da to 1,150,000 Dais present in an amount of about 2% by weight. In some embodiments, hydroxypropyl cellulose is Klucel MF in an amount of about 2% by weight.

In some embodiments of composition (C1a), butylated hydroxytoluene is present in the composition in an amount of from 0.01% to 0.1% or about 0.05% by weight. In some embodiments, butylated hydroxytoluene is present in an amount of from 0.01% to 0.1% by weight. In some embodiments, butylated hydroxytoluene is present in an amount of about 0.5% by weight.

In some embodiments of composition (C1a), phenoxyethanol is present in the composition in an amount of from 0.5% to 2% or about 1% by weight. In some embodiments, phenoxyethanol is present in an amount of from 0.5% to 2% by weight. In some embodiments, phenoxyethanol is present in an amount of about 1% by weight.

In some embodiments of composition (C1a), butylated hydroxytoluene is present in an amount of about 0.5% by weight; and phenoxyethanol is present in an amount of about 1% by weight.

In some embodiments of compositions (A), (B), (C), (A1), (B1), (C1), and (C1a), propylene glycol is a super refined propylene glycol.

In some embodiments of compositions (A), (B), (C), (A1), (B1), (C1), and (C1a), 2-(2-ethoxyethoxy)ethanol is Transcutol HP having a purity of >99.90%.

In some embodiments of compositions (A), (B), (C), (A1), (B1), (C1), and (C1a), the compound is present in the composition in an amount of from 0.5% to 2% or about 1% by weight. In some embodiments, the compound is present in an amount of from 0.5% to 2% by weight, or about 0.1% to about 1% by weight. In some embodiments, the compound is present in an amount of about 1% by weight. In some embodiments, the compound is present in an amount of about 0.10% by weight.

### Forms of Compositions

Topical compositions useful for delivering the compound to a subject (e.g., to the skin of a subject) include, but are not limited to, foams, sprays, aerosols, creams, lotions, ointments, gels, solutions, emulsions, and suspensions. See, e.g., REMINGTON: THE SCIENCE AND PRACTICE OF PHARMACY; (Alfonso R. Gennaro ed. 19th ed. 1995); and Introduction to Pharmaceutical Dosage Forms, 4th ed., Lea & Febiger, Philadelphia (1985). In some embodiments, the topical composition used to deliver the compound is a gel, ointment, lotion, foam or emollient.

In some embodiments, the topical composition used to deliver the compound is a lotion or a cream. Creams and lotions that can be used as topical compositions and their preparation are disclosed in REMINGTON: THE SCIENCE AND PRACTICE OF PHARMACY 282-291 (Alfonso R. Gennaro ed. 19th ed. 1995), the relevant portions of which are hereby incorporated herein by reference.

In some embodiments, the topical composition used to deliver the compound is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the disclosure are disclosed in REMINGTON: THE SCIENCE AND PRACTICE OF PHARMACY 1517-1518 (Alfonso R. Gennaro ed. 19th ed. 1995), which is hereby incorporated herein by reference. Other suitable gels for use with the disclosure are disclosed in U.S. Pat. No. 6,387,383 (issued May 14, 2002), 6,517,847 (issued Feb. 11, 2003), and 6,468,989 (issued Oct. 22, 2002).

In some embodiments, the topical composition used to deliver the compound is an ointment. Ointments are oleaginous semisolids that contain little if any water. In some instances, the ointment is hydrocarbon based, such as a wax, petrolatum, or gelled mineral oil. Suitable ointments for use according to the present disclosure include those disclosed in REMINGTON: THE SCIENCE AND PRACTICE OF PHARMACY 1585-1591 (Alfonso R. Gennaro ed. 19th ed. 1995).

In some embodiments the topical composition used to deliver the compound is an emulsified gel. In some embodiments the topical composition used to deliver the compound is an emulsified spray.

In some embodiments, the topical composition may be achieved in the form of a patch, tape, film, wafer, or bandage including the topical composition as described herein. In some embodiments, the patch, tape, film, wafer, or bandage is in contact with the affected area on the skin. In some embodiments, the patch, tape, film, wafer, or bandage, when applied to a subject, is in contact with areas of the skin of the subject that are adjacent to the affected or target area.

In some embodiments, the topical administration may be achieved in the form of a patch, tape, film, wafer, or bandage including the topical composition as described herein. In some embodiments, the patch, tape, film, wafer, or bandage is in contact with the affected area on the skin. In some embodiments, the patch, tape, film, wafer, or bandage, when applied to a subject, is in contact with areas of the skin of the subject that are adjacent to the affected or target area.

In some embodiments, the patch, tape, film, wafer, or bandage includes an adhesive.

In some embodiments, the topical composition may be applied to a seborrheic keratosis using a tape or film which provides an occluding environment for the Seborrheic Keratosis. The tape or film may be adhesive tape, waterproof adhesive tape, or a plastic film (with or without an adhesive layer). In some embodiments the transparent film used to form an occlusion is Tegaderm.

In some embodiments, the composition is impregnated in the tape or film. In some embodiments, the tape or film is a foam-containing tape or film. Pores or interstitial spaces in the foam can be loaded with the composition as described herein. The foam can have an adhesive layer for adhering the foam to skin. If the foam is porous, an outer non-porous layer can be provided on the back of the foam to enhance tape occlusion.

In some embodiments, the composition may be a form of a slow-release wafer or dot, may also be made by using a porous foam, may have a quantity of the composition placed behind the porous foam, and may have a non-porous backing layer placed behind the composition. The non-porous backing layer can have a portion extending beyond the composition containing Compound 1 and the porous layer. This portion can include a quantity of adhesive for facilitating adhesion of the wafer or dot to the skin of a user peripheral to Seborrheic Keratoses being treated.

In some embodiments the composition for application may be covered by a film or thermosensitive gel material including but not limited to poly(ethylene glycol)/poly(propylene glycol) block copolymers (poloxamers), poly(ethylene glycol)/poly(butylenes glycol) block copolymers, poloxamer-g-poly(acrylic acid) and copolymers of Nisopropylacrylamide that exhibit a sol-to-gel transition in aqueous solutions. In some embodiments the film or thermosensitive gel material may be impregnated with the composition. In some embodiments the film or thermosensitive gel material impregnated with the composition may include but is not limited to poly(ethylene glycol)/poly(propylene glycol) block copolymers (poloxamers), poly(ethylene glycol)/poly(butylenes glycol) block copolymers, poloxamer-g-poly(acrylic acid) and copolymers of Nisopropylacrylamide that exhibit a sol-to-gel transition in aqueous solutions.

In some embodiments, the composition is a pharmaceutical composition. In some embodiments, the composition is a topical composition. In some embodiments, the composition is in the form of a gel, ointment, lotion, foam or emollient. In some embodiments, the composition is a component of a patch, tape, film, wafer, or bandage.

### Methods

In certain aspects, the present disclosure provides a method of treating a skin disease, condition or disorder in a subject in need thereof, the method comprising administering to the subject a composition, as described herein.

In one aspect, the present disclosure provides a method of treating a cutaneous lesion, the method comprising topically administering to the cutaneous lesion a composition comprising:
a compound having the structure:
(i)

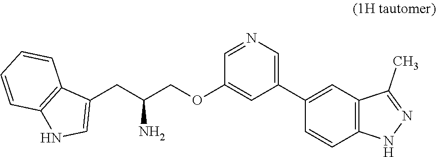 [SMILES: Cc1n[nH]c2ccc(-c3cncc(OC[C@@H](N)Cc4c[nH]c5ccccc45)c3)cc12]

or a salt thereof, and/or
a compound having the structure:
(ii)

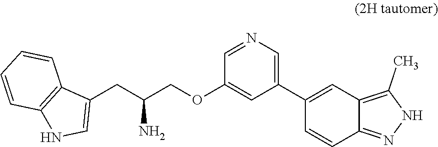 [SMILES: Cc1[nH]nc2ccc(-c3cncc(OC[C@@H](N)Cc4c[nH]c5ccccc45)c3)cc12]

or a salt thereof,
wherein the total amount of (i), (ii), or (i) and (ii) present in the composition is 0.1% to 1% by weight. Alternatively, the total amount of (i), (ii), or (i) and (ii) present in the composition is 0.01% to 10% or 0.01% to 5%.

In some embodiments, the cutaneous lesion is a keratosis. In some embodiments, the keratosis is a seborrheic keratosis. In some embodiments, the composition is administered one, two, three, or four times a day. In some embodiments, the composition is administered one, two, three, or four times a day for about 14 days to about 28 days. In some embodiments, the composition is administered one, two, three, four, or five times a week. In some embodiments, the composition is administered in a cycle comprising 1-4 daily administration for about 1-7 days, followed by no administration for about 1-7 days. In some embodiments, the cycle comprises twice daily administration for about 4 days, followed by no administration for about 4 days. In some embodiments, the cycle is repeated about 2-10 times. In some embodiments, the cycle is repeated about 7 times. In some embodiments, the composition is administered at least once daily for about 28 days (e.g., about once or twice a day). In some embodiments, the composition is administered once daily three times a week. In some embodiments, the composition is administered for a total duration of about 1, 2, 3, 4, 5, or 6 months.

In some embodiments, the method further comprises occlusion of the cutaneous lesion.

In some embodiments, the cutaneous lesion is present on a human subject. In some embodiments, the cutaneous lesion is present on the face, trunk, or an extremity, or a combination thereof, of the human subject.

Physician's Lesion Assessment (PLA) is the assessment of the severity of the target and non-target Seborrheic keratosis at a particular time point. The assessment can be referred to other evaluations (e.g., prior photographs) to assist with these assessments. This PLA assessment is a point scoring system used to assess disease severity. PLA is a four point scoring system comprising a score of 0 to 3. A score of 3 indicates at least 1 mm thickness of seborrheic keratosis, a score of 2 indicates less than 1 mm thickness of seborrheic keratosis, a score of 1 indicates a nearly clear but containing residual surface changes, suggesting of a seborrheic keratosis, and a score of 0 indicates no visible surface changes remained. In some embodiments, treating comprises an improvement of one grade or more in Physician's Lesion Assessment (PLA) score as compared to before administering the composition. In some embodiments, the PLA score prior to administering is at least 2.

In some embodiments, treating comprises reducing the thickness of the cutaneous lesion to less than 1 mm. In some embodiments, the thickness of the cutaneous lesion prior administering is greater than or equal to 1 mm. In some embodiments, the length of the cutaneous lesion prior to administering is from 1 mm to 15 mm, and the width of the cutaneous lesion prior to administering is from 1 mm to 15 mm.

In some embodiments, treating comprises inducing apoptosis of a keratinocyte in the cutaneous lesion.

Terminal deoxynucleotidyl transferase dUTP nick end labeling assay or TUNEL assay is a method for detecting or quantifying cellular apoptosis by labeling the 3′-hydroxyl termini in the double-strand DNA breaks of DNA fragmentation generated during apoptosis. The TUNEL assay utilizes the terminal deoxynucleotidyl transferase (TdT), which is an enzyme that catalyzes attachment of deoxynucleotides, tagged with a fluorochrome or another marker, to 3-hydroxyl termini of DNA double strand breaks. In some embodiments, apoptosis is measured by TUNEL assay.

In some embodiments, the composition is a gel formulation. In some embodiments, the composition comprises an alcohol, a gelling agent, or an antioxidant, or a combination of two or more thereof.

In one aspect, the present disclosure provides a method of treating a skin disease, condition or disorder in a subject in need thereof, comprising administering to the subject the composition described herein.

In some embodiments, the skin disease, condition or disorder is seborrheic keratosis, benign tumor, malignant tumor, parasite, virus of the skin, immune disease or disorder, or a bacterial, fungal or microbial infection. In some embodiments, the skin disease, condition or disorder is seborrheic keratosis. In some embodiments, the skin disease, condition or disorder is the benign tumor, wherein the benign tumor is a benign vascular tumor, benign fibrotic tumor, benign adipocyte tumor, benign sebaceous tumor, benign epidermal tumor, benign melanocytic lesion, or benign neural tumor; the skin disease, condition or disorder is the malignant tumor, wherein the malignant tumor is a malignant melanocytic tumor, malignant epidermal tumor, malignant vascular tumor, malignant metastatic tumor, malignant adipocyte tumor, malignant sebaceous tumor, or malignant fibrotic tumor; the skin disease, condition or disorder is the parasite, wherein the parasite is of the genus Trypanasoma or Lieshmania; the skin disease, condition or disorder is the virus, wherein the virus is molluscum contagiosum virus or human papilloma virus; or the skin disease, condition or disorder is the bacterial, fungal or microbial infection, wherein the bacterial, fungal or microbial infection is otitis media, Staphylococcus aureus infection, Mycobacterium infection, Porphyromonas infection, Salmonella infection, Chlamydia infection, tuberculosis, gingivitis or periodontal disease.

In some embodiments, the composition is applied topically to the head, scalp, face, ear(s), neck, chest, back, inframammary region(s), arm(s), leg(s), intertriginous zone(s), hand(s), foot or feet, or groin. In some embodiments, the composition is administered twice daily. In some embodiments, the composition is administered for about 2 weeks to about 8 weeks. In some embodiments, the composition is administered for about 4 weeks.

In some embodiments, the composition is administered in a pulsed cycle. In some embodiments, the composition is administered under occlusion.

In some embodiments, the skin disease, condition or disorder is skin pigmentation disorder. In some embodiments, the skin pigmentation disorder is Acanthosis nigricans.

In some embodiments, the skin disease, condition or disorder is seborrheic keratosis, benign tumors or skin conditions, malignant tumors, parasites, viruses of the skin, immune diseases or disorders, and a bacterial, fungal or microbial infection.

In some embodiments, the skin disease, condition or disorder is seborrheic keratosis.

In some embodiments, the compositions described herein are useful for treating benign tumors or skin conditions. In some embodiments, the benign tumors are benign vascular tumors, benign fibrotic tumors, benign adipocyte tumors, benign sebaceous tumors, benign epidermal tumors, benign melanocytic lesions, or benign neural tumors. Benign tumors or skin conditions include, but are not limited to, nodules, benign skin tumors, angiomas, hemangiomas, pyogenic granuloma, angiofibroma, tuberous sclerosis complex, angiomyofibroma, angiolipoma, dermatofibroma, fibroma, neurofibromas, scars, scar tissue, keloids, lipomas, acrochordons, melanoacanthoma, acanthoma, clear cell acanthoma, acanthosis nigricans, epidermoid cysts, pilar cysts, dermoid cyst, melanocytic nevi, epidermal nevi, verrucous epidermal nevi, lentigos, café au lait macules, neuroma, schwannoma, and neurolemmoma. In some embodiments, benign tumors are nodules, benign skin tumors, angiomas, hemangiomas, pyogenic granuloma, angiofibroma, tuberous sclerosis complex, angiomyofibroma, angiolipoma, dermatofibroma, fibroma, neurofibromas, scars, scar tissue, keloids, lipomas, acrochordons, melanoacanthoma, acanthoma, clear cell acanthoma, acanthosis nigricans, epidermoid cysts, pilar cysts, dermoid cyst, melanocytic nevi, epidermal nevi, verrucous epidermal nevi, lentigos, café au lait macules, neuroma, schwannoma, or neurolemmoma.

In some embodiments, the compositions described herein are useful for treating malignant tumors. In some embodiments, the malignant tumors are malignant melanocytic tumors, malignant epidermal tumors, malignant vascular tumors, malignant metastatic tumors, malignant adipocyte tumors, malignant sebaceous tumors, or malignant fibrotic tumors. Malignant tumors include, but are not limited to, melanomas, squamous cell carcinoma, keratoacanthoma, actinic keratoses, basal cell carcinomas, angiosarcoma, Kaposi sarcoma, cutaneious breast cancer, Merkel cell cancer, liposarcoma, sebaceoma, sebaceous carcinoma, dermatofibroma sarcoma protuberens, and fibrosarcoma. In some embodiments, malignant tumors are melanomas, squamous cell carcinoma, keratoacanthoma, actinic keratoses, basal cell carcinomas, angiosarcoma, Kaposi sarcoma, cutaneious breast cancer, Merkel cell cancer, liposarcoma, sebaceoma, sebaceous carcinoma, dermatofibroma sarcoma protuberens, or fibrosarcoma.

In some embodiments, the compositions described herein are useful for treating the skin disease, condition or disorder caused by a parasite. In some embodiments, the parasites are parasites of the genus Trypanasoma or Lieshmania. In some embodiments, the parasites are parasites of the genus Leishmania, Endotrypanum, Novymonas, Porcisia, or Zelonia. In some embodiments, the parasite is L. major, L. tropica, L. aethiopica, L. Mexicana, or L. braziliensis. In some embodiments, the disease or condition caused by or associated with the parasite is cutaneous or mucosal or mucocutaneous. In some embodiments, the parasite is Leishmania aethiopica, Leishmania amazonensis, Leishmania arabica, Leishmania aristidesi, Leishmania donovani, Leishmania forattinii, Leishmania gerbilli, Leishmania infantum, Leishmania killicki, Leishmania major, Leishmania Mexicana, Leishmania pifanoi, Leishmania tropica, Leishmania turanica, Leishmania venezeulensis, Leishmania waltoni, Leishmania enriettii, Leishmania macropodum, Leishmania martiniquensis, Leishmania orientalis, Leishmania adleri, Leishmania agamae, Leishmania ceramodactyli, Leishmania gulikae, Leishmania gymnodactyli, Leishmania helioscopi, Leishmania hemidactyli, Leishmania hoogstraali, Leishmania nicollei, Leishmania platycephala, Leishmania phrynocephali, Leishmania senegalensis, Leishmania sofieffi, Leishmania tarentolae, Leishmania zmeevi, Leishmania zuckermani, Leishmania braziliensis, Leishmania guyanensis, Leishmania lainsoni, Leishmania lindenbergi, Leishmania naiffi, Leishmania panamensis, Leishmania peruviana, Leishmania shawi, Leishmania utingensis, Endotrypanum colombiensis, Endotrypanum equatorensis, Endotrypanum herreri, Endotrypanum monterogeii, Endotrypanum schaudinni, Novymonas esmeraldas, Porcisia deanei, Porcisia hertigi, Zelonia australiensis, or Zelonia costaricensis.

In some embodiments, the compositions described herein are useful for treating skin disease, condition or disorder caused by a virus. Viral pathogens include, but are not limited to, adenoviruses, influenza, human herpes virus, Avibimavirus, HIV, and coronaviruses. In some embodiments, the viruses are poxviruses that cause molluscum contagiosum or human papilloma viruses that cause warts.

In some embodiments, the compositions described herein are useful for treating skin disease, condition or disorder associated with bacterial, fungal or microbial infection. In some embodiments, the bacterial, fungal or microbial infection is otitis media, Staphylococcus aureus infection, Mycobacterium infection, salmonellosis, Chlamydia infection, tuberculosis, Porphyromonas infection, gingivitis or periodontal disease.

In some embodiments, the composition described herein is administered to a face of the subject. In some embodiments, the composition described herein is administered to the body of the subject. In some embodiments, the composition described herein is administered topically to the head, scalp, face, ears, neck, chest, back, inframammary regions, arms, legs, groin intertriginous regions, hands, and/or feet. In some embodiments, the composition covers (e.g. occludes) the skin associated with the disease, condition or disorder. As a non-limiting example, the composition covers a seborrheic keratosis.

In some embodiments, the composition described herein is administered to a face of the subject, thereby treating seborrheic keratosis on the face. In some embodiments, the composition described herein is administered to the body of the subject, thereby treating seborrheic keratosis. In some embodiments, the composition described herein is administered topically to one or more areas selected from head, scalp, face, ears, neck, chest, back, inframammary regions, arms, legs, groin, intertriginous regions, hands, and/or feet thereby treating seborrheic keratosis in any one of these areas.

In some embodiments, the skin disease, condition or disorder to be reduced, ameliorated, treated, or prevented is seborrheic keratosis. In some embodiments, the composition described herein is administered in a therapeutically effective amount to achieve partial or complete resolution, involution, or removal of at least one seborrheic keratosis in a subject. In some embodiments, the composition described herein is administered in a therapeutically effective amount to achieve a partial resolution of at least one seborrheic keratosis in a subject. In some embodiments, the composition described herein is administered in a therapeutically effective amount to achieve a complete resolution of at least one seborrheic keratosis in a subject. In some embodiments, the composition described herein is administered in a therapeutically effective amount to achieve an involution of at least one seborrheic keratosis in a subject. In some embodiments, the composition described herein is administered in a therapeutically effective amount to achieve removal of at least one seborrheic keratosis in a subject.

Compositions disclosed herein may be administered as at least a single dose, multiple times a day, daily, multiple times a week, weekly, multiple times a month or monthly. In some embodiments, the composition is administered twice daily. In some embodiments, the composition is administered at least twice weekly. In some embodiments, the composition is administered at least twice monthly. In some embodiments the composition is administered for 2, 3, 4, or five months. In some embodiments the composition is administered in pulsed dosing. In some embodiments the composition is applied twice daily for 2, 3, 4, 5, or 6 days consecutively followed by a pause (e.g. 1, 2, 3, 4, 5, 6, or 7 days) in treatment then followed by application of the composition twice daily for 2, 3, 4, 5, or 6 days consecutively. In some embodiments this pulsed cycle of application is repeated 2, 3, 4, 5, or 6 times. In some embodiments this pulsed cycle of application is repeated up to 15 times. In some embodiments the composition is administered in pulsed dosing or application. In some embodiments the composition is applied once daily for 2, 3, 4, 5, or 6 days consecutively followed by a pause (e.g. 1, 2, 3, 4, 5, 6, or 7 days) in treatment then followed by application of the composition once daily for 2, 3, 4, 5, or 6 days consecutively. In some embodiments this pulsed cycle of application is repeated 2, 3, 4, 5, or 6 times. In some embodiments this pulsed cycle of application is repeated up to 10, 11, 12, 13, 14, or 15 times. In some embodiments, the composition comprising the compound (i.e., Compound 1) in an amount of from 0.01% to 10% by weight is administered as a single dose, multiple times a day, daily, multiple times a week, weekly, twice weekly, multiple times a month, monthly, twice monthly, or in pulsed applications. In some embodiments, the composition comprising the compound (i.e., Compound 1) in an amount of from 0.01% to 5% by weight is administered as a single dose, multiple times a day, daily, multiple times a week, weekly, twice weekly, multiple times a month, monthly, twice monthly, or in pulsed applications. In some embodiments, the composition comprising the compound (i.e., Compound 1) in an amount of from 0.1% to 10% by weight is administered as a single dose, multiple times a day, daily, multiple times a week, weekly, twice weekly, multiple times a month, monthly, twice monthly, or in pulsed applications. In some embodiments, the composition is administered for a total duration of about 1, 2, 3, 4, 5, or 6 months.

In some embodiments, the composition may be formulated at about 0.01% to about 10%, about 0.01%, or about 10% Compound 1 (e.g., Compound 1 1H tautomer and/or Compound 1 2H tautomer) or a salt(s) thereof. In some embodiments, the composition may be formulated at about 0.01% to about 5%, about 0.01%, or about 5% Compound 1 (e.g., Compound 1 1H tautomer and/or Compound 1 2H tautomer) or a salt(s) thereof. In some embodiments, the composition may be formulated at about 0.1% to about 10%, about 0.1%, or about 10% Compound 1 (e.g., Compound 1 1H tautomer and/or Compound 1 2H tautomer) or a salt(s) thereof. In some embodiments, the composition may be formulated at about 0.1% to about 1%, about 0.1%, or about 1% Compound 1 (e.g., Compound 1 1H tautomer and/or Compound 1 2H tautomer) or a salt(s) thereof. In some cases the composition is administered once or twice daily. For example, the composition is administered twice daily. In some cases, the composition is administered for about 2 weeks to about 8 weeks. In some cases, the composition is administered for about 2 weeks. In some cases, the composition is administered for about 4 weeks. In some cases, the composition is administered in a pulsed cycle of application as described herein. In some cases, the composition is administered under occlusion.

In some embodiments, the composition administered is in the form of a gel, ointment, lotion, foam or emollient.

In some embodiments, the composition administered is a component of a patch, tape, film, wafer, or bandage.

In some embodiments, the subject in need thereof is a human.

### Kits

Also provided are kits for use in methods of treatment of a skin disease, condition or disorder where the subject is in need thereof with the composition as described herein in a tube, flexible aluminum tube or laminated plastic tube. The kits can include a topical composition comprising the compound (i.e., Compound 1), a second agent or composition, and instructions providing information to a health care provider regarding usage for treating a skin disease, condition or disorder. Instructions may be provided in printed form or in the form of an electronic or digital medium such as a floppy disc, CD, or DVD, data storage device, flash drive, or in the form of a website address where such instructions may be obtained. A unit dose of a compound or a topical composition provided herein, or a second agent or composition, can include a dosage such that when administered to a subject, a therapeutically or prophylactically effective level of the compound or the topical composition can be maintained at the site of treatment in the subject for at least 1 day.

In another aspect, the present disclosure provides a kit comprising a topical pharmaceutical composition in a tube, flexible aluminum tube or laminated plastic tube, with instructions for use.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### EXAMPLES

### Example 1: Preparation of Compositions

The topical compositions of the present disclosure can be prepared according to the procedure provided below. Reaction conditions, steps and reactants not provided in the procedure below would be apparent to, and known by, those skilled in the art.

Excipients (i.e., C₂₋₆ alcohol, an organic solvent and/or a penetration enhancer, an antioxidant, a preservative, and/or water) were aliquoted or weighted into individual vessels to form a mixture. The compound (i.e., Compound 1) was added to the mixture to achieve a desired concentration. Then the gelling agent (e.g., HPC) were added accordingly. In some of the compositions where a pH adjuster (e.g., citric acid) was used, pH of the formed mixture was adjusted with an aqueous citric acid solution (e.g., 0.1 M, 0.5 M, or 1 M solution) to a desired apparent pH (e.g., about 5.5 to 6.5). In some of compositions, a second addition of water (if present) was finally used to titrate the composition to 100% by weight. The final mixture was then mixed well to form the composition.

### Example 2: Various Compositions with/without Compound 1

The following compositions were prepared according to the general procedure using excipients of Table 1 to Table 7.

TABLE 1
Various Compositions in HPC Gels

| | Compositions (wt/wt %) | | | |
|---|---|---|---|---|
| | Ex. 2A- | Ex. 2A- | Ex. 2A- | Ex. 2A- |
| | 0% | 1% | 0.1% | 0.01% |
| Components | (100 g) | (100 g) | (100 g) | (300 g) |
| Compound 1 | — | 1.00 | 0.10 | 0.01 |
| Anhydrous Alcohol, 200 | 67.00 | 66.00 | 66.90 | 66.99 |
| Proof, USP | | | | |
| Propylene Glycol, USP | 30.00 | 30.00 | 30.00 | 30.00 |
| Klucel MF Pharm (HPC) | 3.00 | 3.00 | 3.00 | 3.00 |
| Total Amount | 100.0 | 100.0 | 100.0 | 100.0 |

TABLE 2
Various Compositions in Sepineo Gels

| | Compositions (wt/wt %) | | |
|---|---|---|---|
| | Ex. 2B-0% | Ex. 2B-1% | Ex. 2B-0.1% |
| Components | (100 g) | (100 g) | (100 g) |
| Compound 1 | — | 1.00 | 0.10 |
| Propylene Glycol, USP | 68.00 | 67.00 | 67.90 |
| Sterile Water for Irrigation, | 30.00 | 30.00 | 30.00 |
| USP | | | |
| Sepineo P600 | 2.00 | 2.00 | 2.00 |
| Total Amount | 100.0 | 100.0 | 100.0 |

TABLE 3
Various Compositions in Anhydrous Sepineo Gels

| | Compositions (wt/wt %) | | | |
|---|---|---|---|---|
| | Ex. 2C- | Ex. 2C- | Ex. 2C- | Ex. 2C- |
| | 0% | 1% | 0.1% | 0.01% |
| Components | (100 g) | (100 g) | (100 g) | (300 g) |
| Compound 1 | — | 1.00 | 0.10 | 0.01 |
| Anhydrous Alcohol, 200 Proof, | 30.00 | 30.00 | 30.00 | 30.00 |
| USP | | | | |
| Propylene Glycol, USP | 66.00 | 65.00 | 65.90 | 65.99 |
| Sepineo P600 | 4.00 | 4.00 | 4.00 | 4.00 |
| Total Amount | 100.0 | 100.0 | 100.0 | 100.0 |

TABLE 4
Various Compositions Containing High Transcutol in HPC Gels

| | Compositions (wt/wt %) | | |
|---|---|---|---|
| | Ex. 2D-0% | Ex. 2D-1% | Ex. 2D-0.1% |
| Components | (300 g) | (300 g) | (300 g) |
| Compound 1 | — | 1.00 | 0.10 |
| Alcohol, 190 Proof, USP | 15.00 | 15.00 | 15.00 |
| Propylene Glycol, USP | 15.00 | 15.00 | 15.00 |
| Sterile Water for Irrigation, | 20.00 | 20.00 | 20.00 |
| USP | | | |
| Transcutol P (2-(2- | 48.00 | 47.00 | 47.90 |
| ethoxyethoxy)ethanol) | | | |
| Klucel MF Pharm (HPC) | 2.00 | 2.00 | 2.00 |
| Total Amount | 100.0 | 100.0 | 100.0 |

TABLE 5
Various Compositions Containing Low Transcutol in HPC Gels

| | Compositions (wt/wt %) | | |
|---|---|---|---|
| | Ex. 2E-0% | Ex. 2E-1% | Ex. 2E-0.1% |
| Components | (300 g) | (300 g) | (300 g) |
| Compound 1 | — | 1.00 | 0.10 |
| PEG200 | 20.00 | 20.00 | 20.00 |
| Alcohol, 190 Proof, USP | 15.00 | 15.00 | 15.00 |
| Propylene Glycol, USP | 15.00 | 15.00 | 15.00 |
| Sterile Water for Irrigation, | 20.00 | 20.00 | 20.00 |
| USP | | | |
| Transcutol P (2-(2- | 28.00 | 27.00 | 27.90 |
| ethoxyethoxy)ethanol) | | | |
| Klucel MF Pharm (HPC) | 2.00 | 2.00 | 2.00 |
| Total Amount | 100.0 | 100.0 | 100.0 |

TABLE 6
Various Compositions Containing in PEG Ointments

| | Compositions (wt/wt %) | | |
|---|---|---|---|
| | Ex. 2F-0% | Ex. 2F-1% | Ex. 2F-0.1% |
| Components | (300 g) | (300 g) | (300 g) |
| Compound 1 | — | 1.00 | 0.10 |
| PEG200 | 39.00 | 39.00 | 39.00 |
| PEG400, NF | 26.00 | 25.00 | 25.90 |
| Glycerin, USP | 5.00 | 5.00 | 5.00 |
| PEG3350 | 30.00 | 30.00 | 30.00 |
| Total Amount | 100.0 | 100.0 | 100.0 |

TABLE 7
Various Compositions Containing PEG/Transcutol in HPC Gels

| | Compositions (wt/wt %) | | |
|---|---|---|---|
| | Ex. 2G-0% | Ex. 2G-1% | Ex. 2G-0.1% |
| Components | (300 g) | (300 g) | (300 g) |
| Compound 1 | — | 1.00 | 0.10 |
| PEG200 | 39.00 | 39.00 | 39.00 |
| PEG400, NF | 21.00 | 21.00 | 21.00 |
| Transcutol P (2-(2- | 38.00 | 37.00 | 37.90 |
| ethoxyethoxy)ethanol) | | | |
| Klucel MF Pharm (HPC) | 2.00 | 2.00 | 2.00 |
| Total Amount | 100.0 | 100.0 | 100.0 |

### Example 3: A 14-Day Dermal Tolerability Study in Gottingen Minipigs

### Summary

The objective of this study was to determine the local skin tolerability of four (4) compositions of Compound 1 and the respective placebos (Sepineo Gels of Table 2, Anhydrous Sepineo Gels of Table 3, High Transcutol Gels of Table 4, or Low Transcutol Gels of Table 5) following once daily or every other day dermal administration for 14 days to Gottingen Minipigs.

### Materials & Methods

The test articles and placebos (Sepineo Gels of Table 2, Anhydrous Sepineo Gels of Table 3, High Transcutol Gels of Table 4, or Low Transcutol Gels of Table 5) were administered on the left side of each animal once daily for 14 days during the study via dermal application. The test articles and placebos were administered on the right side of each animal once every other day, beginning on Day 2 (Days 2, 4, 6, 8, 10, 12, and 14) via dermal application. The dose concentrations of Compound 1 were 0.01% and 0.1% (the first animal/sex) or 0.1% and 1% (the second animal/sex) and were administered at a dose volume of 0.5 mL/site. On the day prior to the first dose administration, the hair was clipped from the back of the animal. Repeated clipping was done as necessary. Care was taken to avoid abrading the skin. Each exposure site was marked at the corners with an indelible marker. The test articles and placebos were distributed to individual sites (6/side), measuring 1.4×1.4-inch each, on the dorsal surface by gentle inunction with a glass stirring rod or appropriate instrument (e.g., stainless steel spatula). The test articles and placebos were applied evenly with a thin, uniform film of the appropriate dosing material over the test sites. The area was not occluded.

The sites were observed for gross signs of irritation (i.e., erythema and edema, lesion) and any other signs of local or systemic effect and ranked on a scale from 0-5 where 0 corresponded to ‘no effect’. Clinical observations unrelated to application site observations were recorded as unscheduled observations if necessary.

Following the Day 14 dose (2 to 4 hours postdose), all animals were euthanized for skin biopsy sample collection. Prior to the collection, the dosing site(s) were gently washed with a 10% soap solution (e.g., Dawn dish soap) followed by a tepid water rinse using a Wypall®, or equivalent. A hose was utilized to facilitate the rinse using a low-pressure stream of tepid tap water; followed by tape stripping to remove any residual composition prior to performing the punch biopsy. Full thickness samples were collected from each dose site (including placebo sites) and a single naïve site per animal via a 5 mm circular or elliptical punch biopsy instrument or by dissection with a surgical blade.

### Results, Interpretations and Conclusions

All compositions and placebos were generally well-tolerated following once daily or once every other day dermal administration for 14 days. All test articles and placebos were generally well-tolerated. The only finding of irritation occurred in male 1002. With daily dosing, very slight erythema was present on site 3 (Ex. 2D-1% of Table 4) on Days 12 to 14 and on site 6 (Ex. 2E-1% of Table 5) on Days 4 and 7 to 14. No edema was present at any time point in any animal.

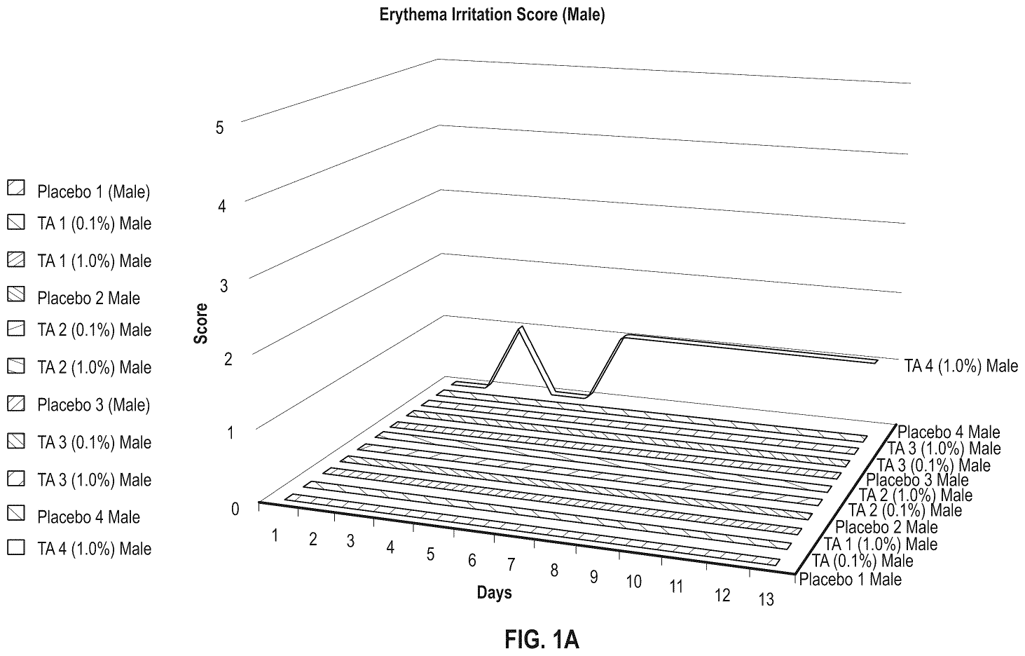

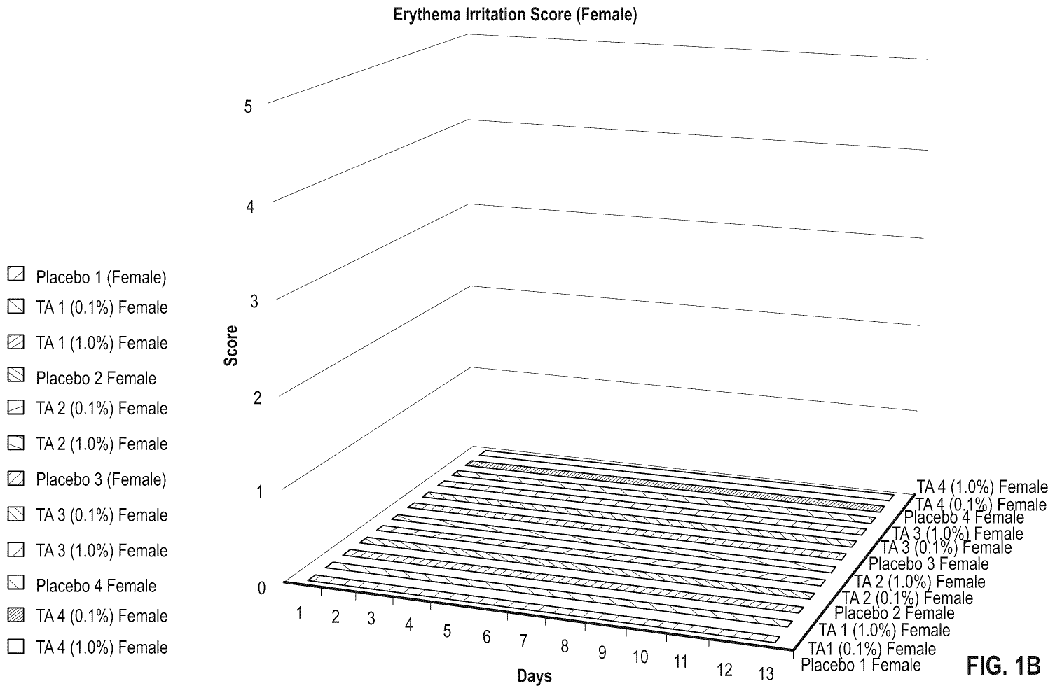

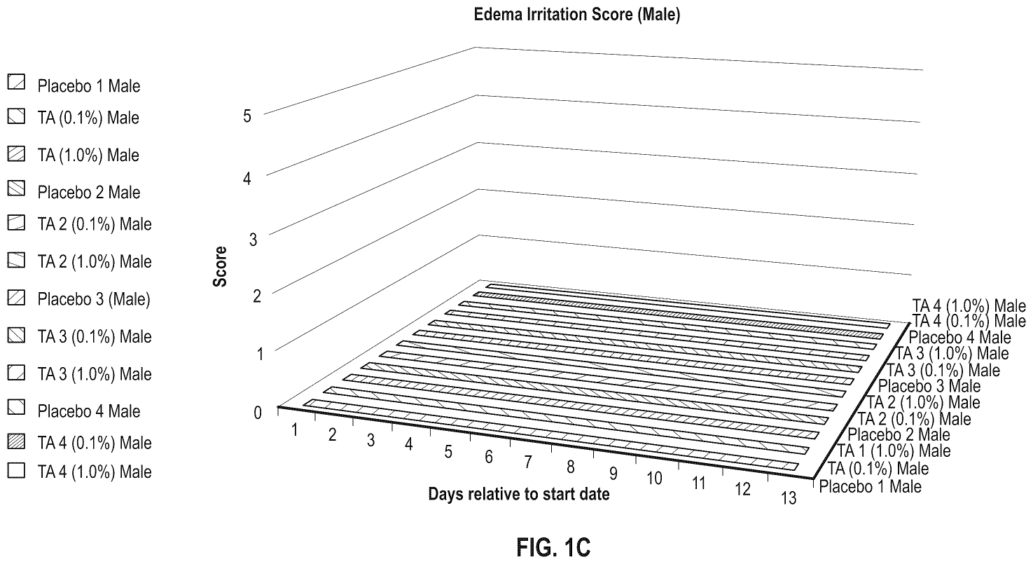

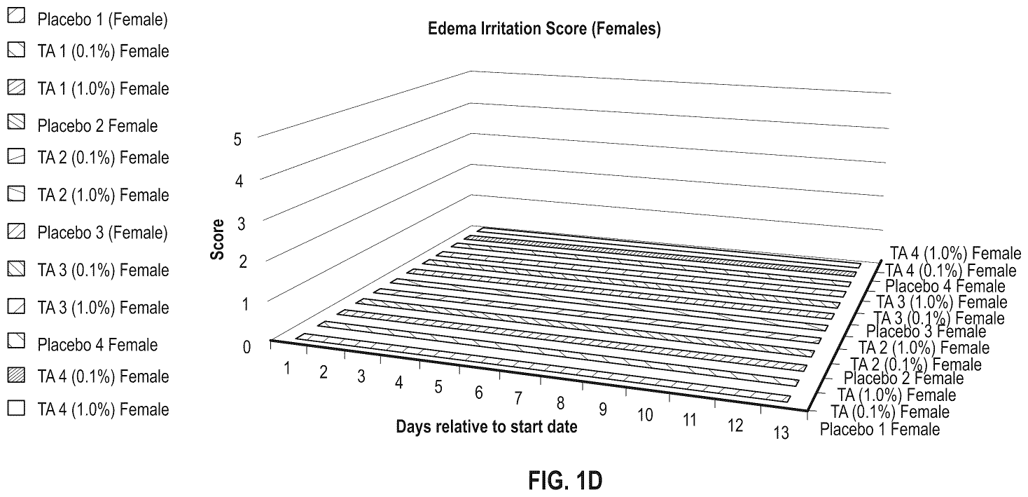

FIG. 1A-FIG. 1D show local skin tolerability of four (4) compositions including Compound 1 and the respective placebos following once daily or every other day dermal administration for 14 days to Gottingen Minipigs, measured by scores of Erythema and Edema. The four (4) compositions correspond to compositions of Table 2 to Table 5 as follows:

| Composition ID in FIG. 1A to FIG. 1D | Composition ID in Example 2 |
|---|---|
| TA 1 (vehicle) | Ex. 2B-0% of Table 2 |
| TA 1 (0.1%) | Ex. 2B-0.1% of Table 2 |
| TA 1 (vehicle) | Ex. 2B-0% of Table 2 |
| TA 1 (1.0%) | Ex. 2B-1% of Table 2 |
| TA 2 (vehicle) | Ex. 2C-0% of Table 3 |
| TA 2 (0.1%) | Ex. 2C-0.1% of Table 3 |
| TA 2 (1.0%) | Ex. 2C-1% of Table 3 |
| TA3 (vehicle) | Ex. 2D-0% of Table 4 |
| TA 3 (0.1%) | Ex. 2D-0.1% of Table 4 |
| TA 3 (1.0%) | Ex. 2D-1% of Table 4 |
| TA 4 (vehicle) | Ex. 2E-0% of Table 5 |
| TA 4 (0.1%) | Ex. 2E-0.1% of Table 5 |
| TA 4 (1.0%) | Ex. 2E-1% of Table 5 |

### Example 4: Further Optimization of Compositions Containing High Transcutol

The compositions of Table 4 that contain about 47-48% of Transcutol P ((2-(2-ethoxyethoxy)ethanol) was further studied with additions of antioxidants (e.g., butylated hydroxytoluene (BHT) and/or butylated hydroxyanisole (BHA), or propyl gallate), preservatives (e.g., phenoxyethanol), and/or a pH adjuster (e.g., aq. citric acid solution). Accordingly, the compositions are shown in Table 8 and Table 9.

TABLE 8
Compositions including Compound 1

| | Compositions (wt/wt %) | | | |
|---|---|---|---|---|
| Components | Ex. 4-1a | Ex. 4-2a | Ex. 4-3a | Ex. 4-4a |
| Compound 1 | 1.06 | 1.06 | 1.06 | 1.06 |
| Alcohol, 200 Proof, USP | 14.25 | 14.25 | 14.25 | 14.25 |
| Propylene Glycol, USP | 15.00 | 15.00 | 15.00 | 15.00 |
| Sterile Water for Irrigation, USP | 20.69 | 9.69 | 9.64 | 9.64 |
| Transcutol P | 47.00 | 47.00 | 47.00 | 47.00 |
| (2-(2-ethoxyethoxy)ethanol) | | | | |
| Propyl Gallate | — | — | — | 0.05 |
| BHT | — | — | 0.05 | — |
| BHA | — | — | — | — |
| Phenoxyethanol | — | 1.00 | 1.00 | 1.00 |
| Aq. Citric Acid | — | adjust to | adjust to | adjust to pH |
| (0.1M/0.5M/1M) | | pH ca. 6.5 | pH ca. 5.5 | ca. 6-6.5 |
| Water 2^{nd} addition | — | Q.S. 100% | Q.S. 100% | Q.S. 100% |
| Klucel MF Pharm (HPC) | 2.00 | 2.00 | 2.00 | 2.00 |
| Total Amount | 100.00 | 100.00 | 100.00 | 100.00 |

| | Compositions (wt/wt %) | | | |
|---|---|---|---|---|
| Components | Ex. 4-1a | Ex. 4-6a | Ex. 4-7a | Ex. 4-8a |
| Compound 1 | 1.06 | 1.06 | 1.06 | 1.06 |
| Alcohol, 200 Proof, USP | 14.25 | 14.25 | 14.25 | 14.25 |
| Propylene Glycol, USP | 15.00 | 15.00 | 15.00 | 15.00 |
| Sterile Water for Irrigation, USP | 20.69 | 19.64 | 9.64 | 9.59 |
| Transcutol P | 47.00 | 47.00 | 47.00 | 47.00 |
| (2-(2-ethoxyethoxy)ethanol) | | | | |
| Propyl Gallate | — | — | — | — |
| BHT | — | 0.05 | 0.05 | 0.05 |
| BHA | — | — | — | 0.05 |
| Phenoxyethanol | — | 1.00 | 1.00 | 1.00 |
| Aq. Citric Acid | — | — | adjust to pH | adjust to pH |
| (0.1M/0.5M/1M) | | | ca. 6-6.5 | ca. 6-6.5 |
| Water 2^{nd} addition | — | — | Q.S. 100% | Q.S. 100% |
| Klucel MF Pharm (HPC) | 2.00 | 2.00 | 2.00 | 2.00 |
| Total Amount | 100.00 | 100.00 | 100.00 | 100.00 |

TABLE 9
Compositions without Compound 1 (Placebo)

| | Compositions (wt/wt %) | | | |
|---|---|---|---|---|
| Components | Ex. 4-1b | Ex. 4-2b | Ex. 4-3b | Ex. 4-4b |
| Alcohol, 200 Proof, USP | 14.25 | 14.25 | 14.25 | 14.25 |
| Propylene Glycol, USP | 15.00 | 15.00 | 15.00 | 15.00 |
| Sterile Water for Irrigation, USP | 21.75 | 10.75 | 10.7 | 10.7 |
| Transcutol P | 47.00 | 47.00 | 47.00 | 47.00 |
| (2-(2-ethoxyethoxy)ethanol) | | | | |
| Propyl Gallate | — | — | — | 0.05 |
| BHT | — | — | 0.05 | — |
| BHA | — | — | — | — |
| Phenoxyethanol | — | 1.00 | 1.00 | 1.00 |
| Aq. Citric Acid | — | adjust to pH | adjust to pH | adjust to pH |
| (0.1M/0.5M/1M) | | ca. 6.5 | ca. 5.5 | ca. 6-6.5 |
| Water 2^{nd} addition | — | Q.S. 100% | Q.S. 100% | Q.S. 100% |
| Klucel MF Pharm (HPC) | 2.00 | 2.00 | 2.00 | 2.00 |
| Total Amount | 100.00 | 100.00 | 100.00 | 100.00 |

| | Compositions (wt/wt %) | | | |
|---|---|---|---|---|
| Components | Ex. 4-1b | Ex. 4-6b | Ex. 4-7b | Ex. 4-8b |
| Alcohol, 200 Proof, USP | 14.25 | 14.25 | 14.25 | 14.25 |
| Propylene Glycol, USP | 15.00 | 15.00 | 15.00 | 15.00 |
| Sterile Water for Irrigation, USP | 21.75 | 20.70 | 10.70 | 10.65 |
| Transcutol P | 47.00 | 47.00 | 47.00 | 47.00 |
| (2-(2-ethoxyethoxy)ethanol) | | | | |
| Propyl Gallate | — | — | — | — |
| BHT | — | 0.05 | 0.05 | 0.05 |
| BHA | — | — | — | 0.05 |
| Phenoxyethanol | — | 1.00 | 1.00 | 1.00 |
| Aq. Citric Acid | — | — | adjust to pH | adjust to pH |
| (0.1M/0.5M/1M) | | | ca. 6-6.5 | ca. 6-6.5 |
| Water 2^{nd} addition | — | — | Q.S. 100% | Q.S. 100% |
| Klucel MF Pharm (HPC) | 2.00 | 2.00 | 2.00 | 2.00 |
| Total Amount | 100.00 | 100.00 | 100.00 | 100.00 |

### Example 5: Induction of Apoptosis in Seborrheic Keratosis

### Summary

A non-clinical ex-vivo human SK explant model was used to test a composition including 1% Compound 1 to induce apoptosis in SK samples. The aim of this study was to investigate the ability of a topically applied Compound to suppress cell viability in SK. The use of human explants allowed for the assay of compositions and the effect of drug product on topical penetration to be assessed in an environment closely mimicking the clinical setting. Shave biopsies of SK were taken from patients undergoing excision of SK for clinical purposes. Pieces of the biopsies, 3-4 mm² in size, were embedded in media and agar for experimentation. Protruding sample surfaces were treated with 2.5 microliter (l) of a composition including 1% Compound 1 (e.g., Ex. 4-6a or Ex. 5-1%), with vehicle only, or left untreated. The explants were then incubated for 72 hours (hr) and fixed for staining and histological assessment. The 72 hr timeframe was used as an efficacy end point and relative apoptosis was assessed by TUNEL staining at the end of the experiment. The resulting images were assessed qualitatively by visual inspection and showed observably more apoptosis in the explants treated with Compound 1 as compared to those treated with vehicle or left untreated. This non-clinical explant study was designed to build confidence in the use of 1% compound 1 for treatment for SK and, indeed, induced an observable increase in apoptosis when applied to SK explants.

### Materials & Methods

Materials required for tissue culture included RPMI 1640 (Thermo Fisher, 11875093), Human serum (GeminiBio #100-110), Amphotericin B (Thermo Fisher, Cat #15290018), and Penicillin Streptomycin (5,000 U/mL, Thermo Fisher, Cat #15140-122), Agarose (Sigma, CAS 9012-36-6) DMSO Sigma Aldrich (#D8418), Transwell Permeable Supports, 12 well plate (Corning 12 mm Transwell® with 3.0 μm PorePolycarbonate Membrane Insert, Sterile Cat #3402).

The composition including Compound 1 and the corresponding vehicle, as shown in Table 10, were used in the study.

TABLE 10
Compositions

| | Compositions (wt/wt %) | |
|---|---|---|
| Components | Ex. 5-1% | Ex. 5-Vehicle |
| Compound 1 | 1.06 | — |
| Alcohol, 200 Proof, USP | 14.25 | 14.25 |
| Propylene Glycol, USP | 15.00 | 15.00 |
| Sterile Water for Irrigation, USP | 19.64 | 19.64 |
| Transcutol P (2-(2-ethoxyethoxy)ethanol) | 47.00 | 47.00 |
| BHT | 0.05 | 0.05 |
| Phenoxyethanol | 1.00 | 1.00 |
| Klucel MF Pharm (HPC) | 2.00 | 2.00 |
| Total Amount | 100.00 | 100.00 |

The ex vivo study was conducted to determine whether Compound 1 in a composition containing a high level of Transcutol (e.g., Ex. 4-6a of Example 4 or Ex. 5-1%) would penetrate a human cutaneous lesion leading to increased apoptosis as measured by TUNEL assay. Biopsies of human seborrheic keratoses were collected and immediately prepared for drug treatment as described in the Experimental Model section.

A total of five (5) seborrheic keratoses were collected from patients under an IRB approved protocol with informed consent. Each SK collected was divided into pieces to be used in different treatment groups. Two separate experiments were performed at different time points. In the first experiment, two keratoses were collected and each was divided in to two pieces. A section of each sample was treated 1% Compound 1 and a section of each was treated with vehicle. The treatment groups were: 1) 1% Compound 1 (N=2); and 2) Vehicle (N=2).

In the second study, three seborrheic keratoses were collected and divided into 3 pieces, each of which were treated with either 1% Compound 1, vehicle only, or were left untreated. The treatment groups were: 1) 1% Compound 1 (N=3); 2) Vehicle (N=3); and 3) Untreated (N=3). In both experiments, either 1% Compound 1 in the composition (Ex. 5-1%) or Vehicle was applied topically to partially submerged tissue samples that were approximately 3.5 mm². Specifically, 2.5 microliter (μl) of the composition of Ex. 5-1% (including 1% Compound 1) was added to the samples, as described above, or 2.5 μl of vehicle was added to the samples as a control. In the second experiment, and additional three pieces of the SKs were left untreated for comparison. Drug product or vehicle was added to each sample topically by pipetting onto the exposed surface area immediately after plating. After a 72 hour incubation at 37° C. and 5% CO₂, the specimen was fixed for 24 hours in 10% formalin and then transferred to 70% ethanol for TUNEL staining.

### Results, Interpretations, and Conclusions

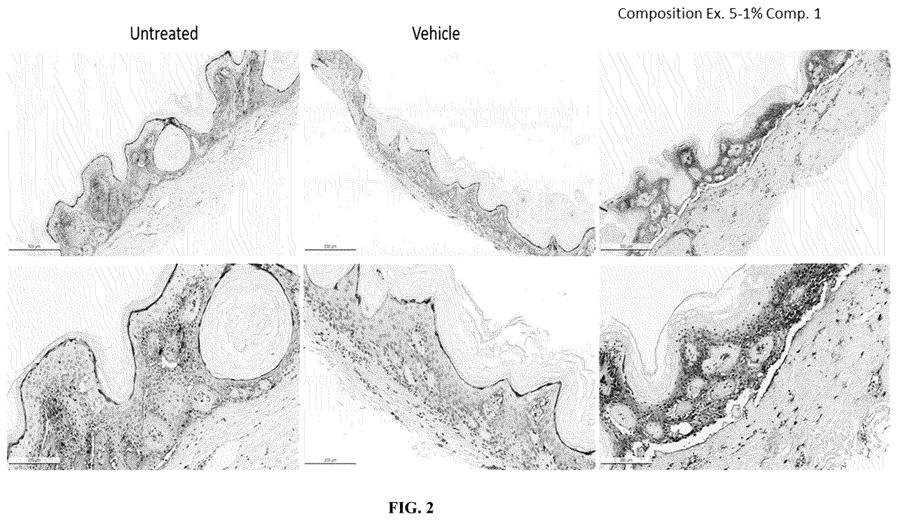

All seborrheic keratosis (n=5) treated with the composition of Ex. 5-1% (including 1% Compound 1) demonstrated a relative increase in apoptosis as measured by TUNEL staining as compared with vehicle (n=5) and untreated samples (n=3). One vehicle treated sample also demonstrated increased apoptosis as compared with untreated but the remaining 4 vehicle treated samples had similar apoptosis levels as compared with untreated controls. FIG. 2 shows explant TUNEL assay images.

It can be concluded that one ex-vivo topical application of the composition (e.g., Ex. 5-1%) can lead to increased apoptosis of the keratinocytes in the seborrheic keratosis 72 hours after application.

### Example 6: Short-Term Stability of Compositions of Example 4

The compositions of Table 8 and Table 9 were subjected to short-term physicochemical stability study following storage up to 8 weeks at 25° C. and 40° C. Parameters evaluated were content and purity of the compound (i.e., Compound 1), apparent pH, macroscopic observations, and microscopic observations.

### Content and Purity of Compound

The content and purity of Compound 1 in compositions following storage up to 8 weeks are detailed in Table 11 and Table 12, respectively (note: only Ex. 4-1a and Ex. 4-6a were further extended to t=8 weeks).

Percentage recovery (0%) compares the response of drug peak to a standard of known concentration whereas percentage peak purity (0 area) compares the area of the drug peak to the sum of the areas of all peaks in the chromatogram. The change in purity (09) of Compound 1 in the compositions following up to 8 weeks of stability testing from t=0 is detailed in Table 13.

TABLE 11
Content of Compound 1 at t = 0 and under Stability Study

Content (wt/wt %)

t = 2 weeks

| Composition ID | t = 0 | 25° C. | 40° C. |
|---|---|---|---|
| Ex. 4-1a | 100.38 | 97.15 | 97.98 |
| (without antioxidant/preservative) | (99.14-101.66) | (95.66-97.99) | (97.71-98.48) |
| Ex. 4-2a | 99.00 | 96.96 | 98.38 |
| (phenoxyethanol, pH 6.5) | (98.09-100.34) | (95.34-97.98) | (97.12-99.34) |
| Ex. 4-3a | 99.50 | 96.37 | 99.36 |
| (BHT/phenoxyethanol, pH 5.5) | (99.23-99.97) | (95.75-97.25) | (98.98-99.94) |
| Ex. 4-4a | 99.15 | 95.74 | 97.99 |
| (propyl gallate/phenoxyethanol, pH 6-6.5) | (98.31-99.84) | (92.58-97.72) | (97.40-98.99) |
| Ex. 4a-6a | 100.31 | 95.29 | 98.84 |
| (with BHT/phenoxyethanol) | (99.41-100.99) | (92.50-99.10) | (97.60-99.77) |
| Ex. 4a-7a | 99.90 | 96.04 | 95.49 |
| (with BHT/phenoxyethanol, pH 6-6.5) | (99.44-100.32) | (95.30-97.41) | (95.00-95.88) |
| Ex. 4a-8a | 99.39 | 95.67 | 96.46 |
| (BHT/BHA/phenoxyethanol, pH 6-6.5) | (99.03-99.94) | (93.20-97.35) | (96.05-96.91) |

| Composition ID | 2-8° C. | 25° C. | 40° C. |
|---|---|---|---|
| | Content (wt/wt %) at t = 4 weeks | | |
| Ex. 4-1a | 99.56 | 100.69 | 99.16 |
| (without antioxidant/preservative) | (98.60-100.44) | (100.52-100.80) | (98.97-99.36) |
| Ex. 4-2a | 99.26 | 98.66 | 97.08 |
| (phenoxyethanol, pH 6.5) | (98.32-100.26) | (98.48-98.85) | (96.49-97.66 |
| Ex. 4-3a | 99.10 | 99.69 | 98.08 |
| (BHT/phenoxyethanol, pH 5.5) | (98.31-100.01) | (99.24-100.49) | (97.05-99.26 |
| Ex. 4-4a | 94.25 | 100.15 | 96.67 |
| (propyl gallate/phenoxyethanol, pH 6-6.5) | (92.62-95.44) | (99.65-101.04) | (95.83-97.14) |
| Ex. 4a-6a | 97.63 | 100.09 | 95.91 |
| (with BHT/phenoxyethanol) | (95.77-99.33) | (99.62-100.66) | (90.97-99.00) |
| Ex. 4a-7a | 96.53 | 97.28 | 96.32 |
| (with BHT/phenoxyethanol, pH 6-6.5) | (95.06-98.75) | (92.82-99.60) | (95.98-96.52) |
| Ex. 4a-8a | 98.96 | 99.66 | 97.31 |
| (BHT/BHA/phenoxyethanol, pH 6-6.5) | (98.11-100.07) | (99.57-99.83) | (96.91-97.72) |
| | Content (wt/wt %) at t = 8 weeks | | |
| Ex. 4-1a | 95.41 | 95.93 | 96.79 |
| (without antioxidant/preservative) | (94.28-97.37) | (95.52-96.56) | (95.88-97.47) |
| Ex. 4a-6a | 96.02 | 95.51 | 95.76 |
| (with BHT/phenoxyethanol) | (94.98-97.47) | (94.36-96.63) | (95.22-96.36) |

TABLE 12
Purity of Compound 1 at t = 0 and under Stability Study

| | Purity (area %) | | |
|---|---|---|---|
| | | t = 2 weeks | |
| Composition ID | t = 0 | 25° C. | 40° C. |
| Ex. 4-1a | 97.64 | 97.73 | 97.51 |
| (without antioxidant/preservative) | | | |
| Ex. 4-2a | 97.92 | 97.65 | 96.72 |
| (phenoxyethanol, pH 6.5) | | | |
| Ex. 4-3a | 97.91 | 97.62 | 96.88 |
| (BHT/phenoxyethanol, pH 5.5) | | | |
| Ex. 4-4a | 97.98 | 97.64 | 96.91 |
| (propyl gallate/phenoxyethanol, pH 6-6.5) | | | |
| Ex. 4a-6a | 97.95 | 97.51 | 97.36 |
| (with BHT/phenoxyethanol) | | | |
| Ex. 4a-7a | 97.98 | 97.58 | 96.87 |
| (with BHT/phenoxyethanol, pH 6-6.5) | | | |
| Ex. 4a-8a | 97.84 | 97.59 | 96.75 |
| (BHT/BHA/phenoxyethanol, pH 6-6.5) | | | |

| Composition ID | 2-8° C. | 25° C. | 40° C. |
|---|---|---|---|
| | Purity (area %) at t = 4 weeks | | |
| Ex. 4-1a | 97.40 | 97.29 | 96.76 |
| (without antioxidant/preservative) | | | |
| Ex. 4-2a | 97.57 | 95.34 | 94.78 |
| (phenoxyethanol, pH 6.5) | | | |
| Ex. 4-3a | 97.63 | 95.41 | 94.77 |
| (BHT/phenoxyethanol, pH 5.5) | | | |
| Ex. 4-4a | 97.68 | 97.45 | 93.85 |
| (propyl gallate/phenoxyethanol, pH 6-6.5) | | | |
| Ex. 4a-6a | 97.59 | 97.50 | 97.08 |
| (with BHT/phenoxyethanol) | | | |
| Ex. 4a-7a | 97.60 | 95.34 | 94.87 |
| (with BHT/phenoxyethanol, pH 6-6.5) | | | |
| Ex. 4a-8a | 97.66 | 97.48 | 94.26 |
| (BHT/BHA/phenoxyethanol, pH 6-6.5) | | | |
| | Purity (area %) at t = 8 weeks | | |
| Ex. 4-1a | 97.34 | 96.93 | 96.24 |
| (without antioxidant/preservative) | | | |
| Ex. 4a-6a | 97.76 | 97.35 | 96.87 |
| (with BHT/phenoxyethanol) | | | |

TABLE 13
Change in Purity of Compound 1 under Stability Study from t = 0

| | | Change in Purity (area %) | |
|---|---|---|---|
| | Purity (area %) | t = 2 weeks | |
| Composition ID | at t = 0 | 25° C. | 40° C. |
| Ex. 4-1a | 97.64 | −0.09 | 0.18 |
| (without antioxidant/preservative) | | | |
| Ex. 4-2a | 97.92 | 0.27 | 1.2 |
| (phenoxyethanol, pH 6.5) | | | |
| Ex. 4-3a | 97.91 | 0.29 | 1.03 |
| (BHT/phenoxyethanol, pH 5.5) | | | |
| Ex. 4-4a | 97.98 | 0.34 | 1.07 |
| (propyl gallate/phenoxyethanol, pH 6-6.5) | | | |
| Ex. 4a-6a | 97.95 | 0.44 | 0.59 |
| (with BHT/phenoxyethanol) | | | |
| Ex. 4a-7a | 97.98 | 0.40 | 1.11 |
| (with BHT/phenoxyethanol, pH 6-6.5) | | | |
| Ex. 4a-8a | 97.84 | 0.25 | 1.09 |
| (BHT/BHA/phenoxyethanol, pH 6-6.5) | | | |

| Composition ID | 2-8° C. | 25° C. | 40° C. |
|---|---|---|---|
| | Change in Purity (area %) at t = 4 weeks | | |
| Ex. 4-1a | 0.24 | 0.40 | 0.88 |
| (without antioxidant/preservative) | | | |
| Ex. 4-2a | 0.35 | 2.58 | 3.14 |
| (phenoxyethanol, pH 6.5) | | | |
| Ex. 4-3a | 0.28 | 2.50 | 3.14 |
| (BHT/phenoxyethanol, pH 5.5) | | | |
| Ex. 4-4a | 0.30 | 0.53 | 4.13 |
| (propyl gallate/phenoxyethanol, pH 6-6.5) | | | |
| Ex. 4a-6a | 0.36 | 0.45 | 0.87 |
| (with BHT/phenoxyethanol) | | | |
| Ex. 4a-7a | 0.38 | 2.64 | 3.11 |
| (with BHT/phenoxyethanol, pH 6-6.5) | | | |
| Ex. 4a-8a | 0.18 | 0.36 | 3.58 |
| (BHT/BHA/phenoxyethanol, pH 6-6.5) | | | |
| | Change in Purity (area %) at t = 8 weeks | | |
| Ex. 4-1a | −0.30 | 0.71 | −1.40 |
| (without antioxidant/preservative) | | | |
| Ex. 4a-6a | −0.19 | −0.60 | −1.08 |
| (with BHT/phenoxyethanol) | | | |

The content of Compound 1 in all seven aqueous gels was within a value of 95-105% from t=0 to 2 weeks. However, following 4 weeks of storage, there was a decrease in drug recovery up to 5% in Ex. 4-4a (94.25% at t=4 weeks, 2-8° C.) and Ex. 4-6a (95.91% at t=4 weeks, 40° C.). At t=0, the purity of Compound 1 in all seven aqueous gels were >97 area %. Following 4 weeks of storage, the drug purity of the compositions decreased by approximately 0.36-2.64 area % at 25° C. and 0.88-4.13 area % area at 40° C. The decrease in drug purity was lower (0.18-0.38 area %) in the compositions stored at 2-8° C. Relative purity of Compound 1 in all seven aqueous gels was maintained in >95% for up to 4 weeks at both 25° C. and 40° C. Ex. 4-4a (without a preservative, an antioxidant, and a pH adjuster) and Ex. 4-6a (based on Ex. 4-4a with BHT) were stable for up to 8 weeks at both 25° C. (<1 area % decrease of purity) and 40° C. (<1.5 area % decrease of purity). The addition of antioxidant and preservative (without adjusting a pH) appeared to further stabilize the composition (see Ex. 4-6a).

### Apparent pH

The apparent pH of compositions with or without Compound 1 at t=0 and 4 weeks (2-8° C., 25° C., and 40° C.) is presented in Table 14.

TABLE 14
Apparent pH of Compositions under Stability Study

| | Apparent pH | | | | | |
|---|---|---|---|---|---|---|
| | | t = 2 weeks | | t = 4 weeks | | |
| Composition ID | t = 0 | 25° C. | 40° C. | 2-8° C. | 25° C. | 40° C. |
| Ex. 4-1a | 9.13 | 9.23 | 9.25 | 9.21 | 9.15 | 9.11 |
| (without antioxidant/preservative) | | | | | | |
| Ex. 4-1b (vehicle) | 8.11 | 7.7 | 7.34 | 8.09 | 7.26 | 7.51 |
| Ex. 4-2a | 6.53 | 6.65 | 6.61 | 6.56 | 6.51 | 6.49 |
| (phenoxyethanol, pH 6.5) | | | | | | |
| Ex. 4-2b (vehicle) | 6.70 | 6.77 | 6.79 | 6.86 | 6.94 | 6.64 |
| Ex. 4-3a | 5.43 | 5.53 | 5.58 | 5.52 | 5.54 | 5.48 |
| (BHT/phenoxyethanol, pH 5.5) | | | | | | |
| Ex. 4-3b (vehicle) | 5.92 | 6.01 | 5.98 | 6.01 | 6.04 | 5.98 |
| Ex. 4-4a | 6.37 | 6.33 | 6.4 | 6.37 | 6.31 | 6.3 |
| (propyl gallate/phenoxyethanol, pH 6-6.5) | | | | | | |
| Ex. 4-4b (vehicle) | 6.27 | 6.59 | 6.44 | 6.01 | 6.04 | 5.98 |
| Ex. 4a-6a | 9.42 | 9.21 | 9.13 | 8.94 | 8.82 | 8.78 |
| (with BHT/phenoxyethanol) | | | | | | |
| Ex. 4-6b (vehicle) | 7.83 | 7.87 | 7.6 | 6.49 | 6.46 | 6.26 |
| Ex. 4a-7a | 6.50 | 6.45 | 6.47 | 6.39 | 6.40 | 6.37 |
| (with BHT/phenoxyethanol, pH 6-6.5) | | | | | | |
| Ex. 4-7b (vehicle) | 6.82 | 6.89 | 6.76 | 8.27 | 8.06 | 7.67 |
| Ex. 4a-8a | 6.37 | 6.33 | 6.35 | 6.29 | 6.25 | 6.16 |
| (BHT/BHA/phenoxyethanol, pH 6-6.5) | | | | | | |
| Ex. 4-8b (vehicle) | 6.55 | 6.65 | 6.54 | 6.52 | 6.54 | 6.52 |

At t=0, the apparent pH of the compositions with pH adjuster ranged from 5.94-7.67 with no notable (±>1 pH unit) difference in pH between the placebo and corresponding active compositions. Following 4 weeks of storage, the pH values ranged from 5.52-6.86 (2-8° C.), 5.54-8.06 (25° C.) and 5.48-7.67 (40° C.), in line with the pH values are t=0. The pH of gel compositions which were not adjusted for pH remained at about pH 9: Ex. 4-1a (active) (9.13 at t=0, 9.21 at t=4 weeks, 2-8° C., 9.15 at t=4 weeks, 25° C., and 9.11 at t=4 weeks, 40° C.); and Ex. 4-6a (active) (9.42 at t=0, 8.94 at t=4 weeks, 2-8° C., 8.82 at t=4 weeks, 25° C., and 8.79 at t=4 weeks, 40° C.).

### Macroscopic Observations

The macroscopic appearance and macroscopic images against light and dark backgrounds were accessed for all seven compositions including Compound 1 and corresponding vehicles.

At t=0, all compositions (both active and vehicles) were clear, had medium viscosity and smooth application. The placebo compositions appeared colorless whereas the active compositions had a light beige coloration. There were no obvious changes to the compositions when stored for t=4 weeks at 2-8° C., 25° C., and 40° C. except slight discoloration observed in Ex. 4-4b (vehicle) at t=4 weeks (40° C.), where propyl gallate was present in the composition.

### Microscopic Observations

The microscopic appearance and microscopic images (non-polarised and polarised light) were accessed for all seven compositions including Compound 1 and corresponding vehicles.

There was no evidence of drug particulates in all compositions (both active and vehicles) over the experimental duration (t=0 to 4 weeks, 2-8° C., 25° C., and 40° C.). Though most of the studied compositions were free of excipients particulates, at t=0 and 2 weeks, excipient particulates were observed in three placebo compositions (Ex. 4-4b, Ex. 4-7b, and Ex. 4-8b) and Ex. 4-8a (active). At t=4 weeks, excipient particulates were observed in Ex. 4-1a (active) (40° C.), Ex. 4-1b (vehicle) (25° C.), Ex. 4-2a (active) and Ex. 4-2b (vehicle) (2-8° C.), Ex. 4-4a (active) (40° C.), and Ex. 4-7a (active) (t=2 weeks and 4 weeks, 25 and 40° C.). It should be noted that the morphology of the particulates was not consistent across the time points and could potentially be un-hydrated gelling agent (HPC), indicating that longer hydration time would be required for the gelling agent, which would be further optimized during process development.

Compositions Ex. 4-6a (active) and Ex. 4-6b (vehicle) were observed to be free of both drug and excipients particulates over the experimental duration (t=0 to 4 weeks, 2-8° C., 25° C., and 40° C.).

### Viscosity

Viscosity of Ex. 4-4a (1.00 wt/wt % Compound 1) (as Ex. 6-4a-1%), the same composition (0.10 wt/wt % Compound 1) (as Ex. 6-4a-0.1%), and Ex. 4-4b (vehicle) (as Ex. 6-4b) was monitored for a period of 3 months, as shown in Table 15.

TABLE 15
Viscosity of Compositions under Stability Study

| | | Apparent viscosity (cP) | | | | |
|---|---|---|---|---|---|---|
| | | | t = 1 month | | t = 3 month | |
| | | | 25° C./60% | 40° C./75% | 25° C./60% | 40° C./75% |
| Composition | Pack | t = 0 | RH | RH | RH | RH |
| Ex. 6-4a-1% | Vial | 20,240 | 20,320 | 21,360 | 26,280 | 25,440 |
| | Tube | | 20,720 | 20,720 | 26,080 | 27,760 |
| Ex. 6-4a-0.1% | Vial | 21,880 | 20,880 | 20,080 | 24,480 | 25,320 |
| | Tube | | 22,560 | 20,280 | 24,840 | 25,320 |
| Ex. 6-4b | Vial | 20,160 | 21,240 | 20,000 | 21,680 | 25,800 |
| | Tube | | 21,800 | 21,040 | 23,320 | 25,920 |

### Example 7: In Human Demonstration of Topical Applications for Treatment of Seborrheic Keratosis

### Summary

Promising in vitro and explant studies prompted further investigation to the benefits of Composition Ex. 5—Compound 1 in human clinical studies for the treatment of SKs. This study is a first-in-human open label adaptive design trial to explore the safety and efficacy of Composition Ex. 5—Compound 1 at a concentration of 1.0% or 0.10% with excipient topical gel in patients with Seborrheic Keratosis (SK).

### Methods

The study enrolls up to 35 subjects with 4 SK Target Lesions (SKTSs) each, across six cohorts with different treatment regimens. The duration of subject participation was approximately 12-16 weeks.

The 3 completed cohorts all had trunkal SKs that were treated with Composition Ex. 5—Compound 1: Cohort 1 was treated with 1.0% twice daily (BID) dosing for 14 days; Cohort 2 was treated with 1.0% BID for 28 days; and Cohort 3 was treated with 1.0% pulsed-BID 4 days on/4 days off over 28 days. All subjects were followed for a minimum of 4 weeks after the last dose at which time all four SKTL lesions were scored using the PLA scale. Forty three lesions were included for primary and secondary analysis. As defined in the protocol, 17 lesions were excluded; 3 were biopsied as non-responsive SKs without 4 weeks of post-treatment follow up, and 14 were histologically confirmed as not being SKs (6 nevi, 4 verrucous keratoses, and 4 lentigos).

Primary and secondary efficacy endpoints were based on PLA scoring, a 4 point scale with a 3 indicating a >1 mm thickness SK, a 2 with <1 mm thickness SK, a 1 with nearly clear but residual surface changes suggestive of an SK and 0 with no visible surface changes remaining.

### Results, Interpretations, and Conclusions

All three cohorts combined showed 81% of lesions had at least 1 point improvement in Physician Lesion Assessment (PLA) score, 74% of SK lesions were clear (PLA of 0) or nearly clear (PLA of 1), and 44% lesion were completely clear (PLA of 0). The study showed early dose response efficacy, whereby the 28 day treatment regimens, Cohorts 2 and 3, showed the highest percentage of improvements in SKTLs: 100% SKTLs with at least one PLA improvement for both cohorts, 79% and 100% clear or nearly clear, and 57% and 46% completely clear, respectively. The treatment was well tolerated with no serious adverse events or adverse events and only mild (16.7%) or moderate (3.3%) irritation in a small percentage of lesions.

TABLE 16
Results from first three cohorts treated with Composition Ex. 5 - Compound 1

| | Cohort 1 | Cohort 2 | Cohort 3 | |
|---|---|---|---|---|
| | (5 pts. | (5 pts. | (4 at pulsed 4 | |
| | at BID | at BID | on/4 off over | All Cohorts |
| | 14 day) | 28 day) | 28 day) | (n = 14) |
| PLA 0: SK lesions | 31% (5/16) | 57% (8/14) | 46% (6/13) | 44% (19/43) |
| clear | PI 14-56% | PI 32-79% | PI 23-71% | PI 30-59% |
| SK lesions clear or | 31% (5/16) | 57% (8/14) | 54% (7/13) | 47% (20/43) |
| nearly clear AND at | | | | |
| least 2 grades | | | | |
| improvement PLA | | | | |
| PLA 0 or 1: SK lesions | 50% (8/16) | 79% (11/14) | 100% (13/13) | 74% (32/43) |
| clear or nearly clear | | | | |
| Primary Endpoint: SK | 50% (8/16) | 100% (14/14) | 100% (13/13) | 81% (35/43) |
| lesions with at least 1 | | | | |
| point improvement PLA | | | | |
| 80 | | | | |

Note:
PI = probability index

TABLE 17a
The number and percentage of lesions with at least 1 unit improvement
in PLA scores from baseline to 4 weeks post treatment.

| | | | Percentage of | |
|---|---|---|---|---|
| | Number of lesions | | lesions with at least | |
| | with at least 1 unit | Total | 1 unit improvement | Bayesian 95% |
| | improvement from | number of | from baseline to | Predictive |
| | baseline to week 4 | lesions | week 4 | Interval |
| All 3 cohorts | 34 | 43 | 79% | 65%-89% |
| Cohort 1 (28 | 8 | 16 | 50% | 28%-72% |
| doses over 14 | | | | |
| days) | | | | |
| Cohort 2 (56 | 13 | 14 | 93% | 68%-98% |
| doses over 28 | | | | |
| days) | | | | |
| Cohort 3 (32 | 13 | 13 | 100% | 77%-100% |
| doses over 4 | | | | |
| cycles over 28 | | | | |
| days) | | | | |

TABLE 17b
The number and percentage of lesions with at least
1 unit improvement in PLA scores by last visit.

| | Number of lesions | | Percentage of | |
|---|---|---|---|---|
| | with at least 1 unit | Total number of | lesions with at least | |
| | improvement from | lesions with | 1 unit improvement | Bayesian 95% |
| | baseline by last | follow-up by | from baseline by | Predictive |
| | visit | last visit | last visit | Interval |
| All 3 cohorts | 35 | 143 | 81% | 67%-90% |
| Cohort 1 (28 | 8 | 16 | 50% | 28%-72% |
| doses over 14 | | | | |
| days) | | | | |
| Cohort 2 (56 | 14 | 14 | 100% | 78%-100% |
| doses over 28 | | | | |
| days) | | | | |
| Cohort 3 (32 | 13 | 13 | 100% | 77%-100% |
| doses over 4 | | | | |
| cycles over 28 | | | | |
| days) | | | | |

TABLE 17c
The number and percentage of lesions with at least
1 unit improvement in PLA scores by end of study.

| | Number of lesions | | Percentage of | |
|---|---|---|---|---|
| | with at least 1 unit | Total number of | lesions with at least | |
| | improvement from | lesions with | 1 unit improvement | Bayesian 95% |
| | baseline by end of | follow-up by | from baseline by | Predictive |
| | study | end of study | end of study | Interval |
| All 3 cohorts | 30 | 30 | 100% | 89%-100% |
| Cohort 1 (28 | 6 | 6 | 100% | 59%-100% |
| doses over 14 | | | | |
| days) | | | | |
| Cohort 2 (56 | 11 | 11 | 100% | 74%-100% |
| doses over 28 | | | | |
| days) | | | | |
| Cohort 3 (32 | 13 | 13 | 100% | 77%-100% |
| doses over 4 | | | | |
| cycles over 28 | | | | |
| days) | | | | |

TABLE 18a
The number and percentage of lesions that are clear
(0) or nearly clear (1) at 4 weeks post treatment.

| | Number of | | Percentage of | |
|---|---|---|---|---|
| | lesions | | lesions | |
| | that are | | that are | |
| | clear (0) | | clear (0) | |
| | or nearly | | or nearly | |
| | clear (1) | Total | clear (1) | Bayesian 95% |
| | at week | number of | at week | Predictive |
| | 4. | lesions | 4. | Interval |
| All 3 cohorts | 31 | 43 | 71% | 57%-83% |
| Cohort 1 | 8 | 16 | 50% | 28%-72% |
| Cohort 2 | 10 | 14 | 71% | 45%-88% |
| Cohort 3 | 13 | 13 | 100% | 77%-100% |

TABLE 18b
The number and percentage of lesions that are clear (0) or nearly clear (1) 4
weeks post treatment; and have at least 2 grades of improvement from baseline.

| | | | Percentage of | |
|---|---|---|---|---|
| | Number of lesions | | lesions that are clear | |
| | that are clear (0) or | | (0) or nearly clear | |
| | nearly clear (1) at | | (1) at week 4; and | |
| | week 4; and have at | | have at least 2 | |
| | least 2 grades of | Total | grades of | Bayesian 95% |
| | improvement from | number of | improvement from | Predictive |
| | baseline. | lesions | baseline. | Interval |
| All 3 cohorts | 13 | 43 | 30% | 19%-45% |
| Cohort 1 | 1 | 16 | 6% | 1%-29% |
| Cohort 2 | 6 | 14 | 43% | 21%-68% |
| Cohort 3 | 6 | 13 | 46% | 19%-45% |

TABLE 18c
The number and percentage of lesions that
are clear (0) at 4 weeks post treatment.

| | Number of | | Percentage of | |
|---|---|---|---|---|
| | lesions | | lesions | |
| | that are | | that are | |
| | clear (0) | Total | clear (0) | Bayesian 95% |
| | at week | number of | at week | Predictive |
| | 4. | lesions | 4. | Interval |
| All 3 cohorts | 11 | 43 | 27% | 16%-42% |
| Cohort 1 | 1 | 16 | 6% | 1%-29% |
| Cohort 2 | 6 | 14 | 43% | 21%-68% |
| Cohort 3 | 4 | 13 | 31% | 13%-58% |

TABLE 18d
The number and percentage of lesions that are
clear (0) or nearly clear (1) by last visit.

| | Number of | | Percentage of | |
|---|---|---|---|---|
| | lesions | | lesions | |
| | that are | | that are | |
| | clear (0) | | clear (0) | |
| | or nearly | | or nearly | |
| | clear (1) | Total | clear (1) | Bayesian 95% |
| | by last | number of | by last | Predictive |
| | visit. | lesions | visit. | Interval |
| All 3 cohorts | 32 | 43 | 74% | 60%-85% |
| Cohort 1 | 8 | 16 | 50% | 28%-72% |
| Cohort 2 | 11 | 14 | 79% | 52%-92% |
| Cohort 3 | 13 | 13 | 100% | 77%-100% |

TABLE 18e
The number and percentage of lesions that are clear (0) or nearly clear (1)
by last visit; and have at least 2 grades of improvement from baseline.

| | | | Percentage of | |
|---|---|---|---|---|
| | Number of lesions | | lesions that are clear | |
| | that are clear (0) or | | (0) or nearly clear | |
| | nearly clear (1) by | | (1) by last visit; and | |
| | last visit; and have | | have at least 2 | |
| | at least 2 grades of | Total | grades of | Bayesian 95% |
| | improvement from | number of | improvement from | Predictive |
| | baseline. | lesions | baseline. | Interval |
| All 3 cohorts | 20 | 43 | 47% | 32%-61% |
| Cohort 1 | 5 | 16 | 31% | 14%-56% |
| Cohort 2 | 8 | 14 | 57% | 32%-79% |
| Cohort 3 | 7 | 13 | 54% | 29%-77% |

TABLE 18f
The number and percentage of lesions
that are clear (0) by last visit.

| | Number of | | Percentage of | |
|---|---|---|---|---|
| | lesions | | lesions | |
| | that are | | that are | |
| | clear (0) | Total | clear (0) | Bayesian 95% |
| | by last | number of | by last | Predictive |
| | visit. | lesions | visit. | Interval |
| All 3 cohorts | 19 | 43 | 44% | 30%-59% |
| Cohort 1 | 5 | 16 | 31% | 14%-56% |
| Cohort 2 | 8 | 14 | 57% | 32%-79% |
| Cohort 3 | 6 | 13 | 46% | 23%-71% |

TABLE 18g
The number and percentage of lesions that are
clear (0) or nearly clear (1) by end of study.

| | Number of | | Percentage of | |
|---|---|---|---|---|
| | lesions | Total | lesions | |
| | that are | number of | that are | |
| | clear (0) | lesions | clear (0) | |
| | or nearly | with | or nearly | |
| | clear (1) | follow-up | clear (1) | Bayesian 95% |
| | by end | by end | by end of | Predictive |
| | of study. | of study | study. | Interval |
| All 3 cohorts | 30 | 30 | 100% | 89%-100% |
| Cohort 1 | 6 | 6 | 100% | 59%-100% |
| Cohort 2 | 11 | 11 | 100% | 74%-100% |
| Cohort 3 | 13 | 13 | 100% | 77%-100% |

TABLE 18h
The number and percentage of lesions that are clear (0) or nearly clear (1);
and have at least 2 grades of improvement from baseline, by end of study.

| | Number of lesions | | Percentage of | |
|---|---|---|---|---|
| | that are clear (0) or | | lesions that are clear | |
| | nearly clear (1) by | | (0) or nearly clear | |
| | end of study; and | | (1) by end of study; | |
| | have at least 2 | Total number of | and have at least 2 | |
| | grades of | lesions with | grades of | Bayesian 95% |
| | improvement from | follow-up by | improvement from | Predictive |
| | baseline. | end of study | baseline. | Interval |
| All 3 cohorts | 19 | 30 | 63% | 45%-78% |
| Cohort 1 | 5 | 6 | 83% | 42%-96% |
| Cohort 2 | 8 | 11 | 73% | 43%-90% |
| Cohort 3 | 7 | 13 | 54% | 29%-79% |

TABLE 18i
The number and percentage of lesions that are clear (0) by end of study.

| | Number of | Total number of | Percentage of | |
|---|---|---|---|---|
| | lesions that are | lesions with | lesions that are | Bayesian 95% |
| | clear (0) by end | follow-up by end | clear (0) by end of | Predictive |
| | of study. | of study | study. | Interval |
| All 3 cohorts | 20 | 30 | 67% | 49%-81% |
| Cohort 1 | 5 | 6 | 83% | 42%-96% |
| Cohort 2 | 8 | 11 | 73% | 43%-90% |
| Cohort 3 | 6 | 13 | 46% | 23%-71% |

TABLE 19a
The number and percentage of subjects that are clear (0) in all
4 lesions or in at least 3 lesions at 4 weeks post treatment.

| | Number of | Number of |
|---|---|---|
| | subjects | subjects |
| | that are | that are |
| | clear (0) | clear (0) |
| | in all 4 | in at least |
| | lesions at | 3 lesions at |
| | week 4. | week 4. |
| All 3 cohorts | 0 | 2 |
| Cohort 1 | 0 | 0 |
| Cohort 2 | 0 | 1 |
| Cohort 3 | 0 | 1 |

TABLE 19b
The number and percentage of subjects that are clear (0)
in all 4 lesions or in at least 3 lesions by last visit.

| | Number of | Number of |
|---|---|---|
| | subjects | subjects |
| | that are | that are |
| | clear (0) | clear (0) |
| | in all 4 | in at least |
| | lesions | 3 lesions |
| | by last | by last |
| | visit. | visit |
| All 3 cohorts | 0 | 4 |
| Cohort 1 | 0 | 1 |
| Cohort 2 | 0 | 2 |
| Cohort 3 | 0 | 1 |

TABLE 19c
The number and percentage of subjects that are clear (0) in
all 4 lesions or in at least 3 lesions by end of study.

| | Number of | Number of |
|---|---|---|
| | subjects | subjects |
| | that are | that are |
| | clear (0) | clear (0) |
| | in all 4 | in at least |
| | lesions | 3 lesions |
| | by end | by end |
| | of study. | of study. |
| All 3 cohorts | 0 | 4 |
| Cohort 1 | 0 | 1 |
| Cohort 2 | 0 | 2 |
| Cohort 3 | 0 | 1 |

TABLE 20
Between the lesions with baseline PLA score 2 and baseline
PLA score 3, the lesions with PLA scores of ≤1 and at
least two grades of improvements at 4 weeks post treatment.

| | | Number of | | Percentage of | |
|---|---|---|---|---|---|
| | | lesions with PLA | | lesions with PLA | |
| | | scores of ≤1 and | | scores of ≤1 and | |
| | | at least two | | at least two | |
| | | grades of | Total | grades of | Bayesian 95% |
| | | improvements at | number of | improvements at | Predictive |
| | | week 4 | lesions | week 4 | Interval |
| Baseline | All 3 | 9 | 34 | 28% | 16%-46% |
| PLA = 2 | cohorts | | | | |
| | Cohort 1 | 1 | 14 | 7% | 2%-32% |
| | Cohort 2 | 5 | 10 | 50% | 23%-77% |
| | Cohort 3 | 3 | 10 | 30% | 11%-61% |
| Baseline | All 3 | 4 | 9 | 44% | 19%-74% |
| PLA = 3 | cohorts | | | | |
| | Cohort 1 | 0 | 2 | 0% | 0%-71% |
| | Cohort 2 | 1 | 4 | 25% | 5%-72% |
| | Cohort 3 | 3 | 3 | 100% | 40%-100% |

TABLE 21a
PLA scores of Cohorts 1, 2, 3 lesions at various time points between the baseline and Day 56.

| | | | Days | | | | | | | | | | | | | |
|---|---|---|---|---|---|---|---|---|---|---|---|---|---|---|---|---|
| | Baseline | | 7 | | 14 | | 21 | | 28 | | 35 | | 42 | | 56 | |
| PLA 0 | 0 | (0.0%) | 0 | (0.0%) | 0 | (0.0%) | 0 | (0.0%) | 1 | (2.4%) | 3 | (11.1%) | 5 | (11.6%) | 11 | (35.5%) |
| PLA 1 | 0 | (0.0%) | 0 | (0.0%) | 3 | (7.0%) | 17 | (39.5%) | 22 | (52.4%) | 19 | (70.4%) | 25 | (58.1%) | 16 | (51.6%) |
| PLA 2 | 34 | (79.1%) | 37 | (86.0%) | 35 | (81.4%) | 24 | (55.8%) | 17 | (40.5%) | 5 | (18.5%) | 11 | (25.6%) | 4 | (12.9%) |
| PLA 3 | 9 | (20.9%) | 6 | (14.0%) | 5 | (11.6%) | 2 | (4.7%) | 2 | (4.8%) | 0 | (0.0%) | 2 | (4.7%) | 0 | (0.0%) |

TABLE 21b
PLA scores of Cohorts 2 & 3 lesions at various time points between the baseline and Day 56.

| | | | Days | | | | | | | | | | | | | |
|---|---|---|---|---|---|---|---|---|---|---|---|---|---|---|---|---|
| | Baseline | | 7 | | 14 | | 21 | | 28 | | 35 | | 42 | | 56 | |
| PLA 0 | 0 | (0.0%) | 0 | (0.0%) | 0 | (0.0%) | 0 | (0.0%) | 1 | (3.8%) | 3 | (11.1%) | 4 | (14.8%) | 10 | (40.0%) |
| PLA 1 | 0 | (0.0%) | 0 | (0.0%) | 0 | (0.0%) | 15 | (55.6%) | 18 | (69.2%) | 19 | (70.4%) | 18 | (66.7%) | 11 | (44.0%) |
| PLA 2 | 20 | (74.1%) | 23 | (85.2%) | 24 | (88.9%) | 12 | (44.4%) | 7 | (26.9%) | 5 | (18.5%) | 5 | (18.5%) | 4 | (16.0%) |
| PLA 3 | 7 | (25.9%) | 4 | (14.8%) | 3 | (11.1%) | 0 | (0.0%) | 0 | (0.0%) | 0 | (0.0%) | 0 | (0.0%) | 0 | (0.0%) |

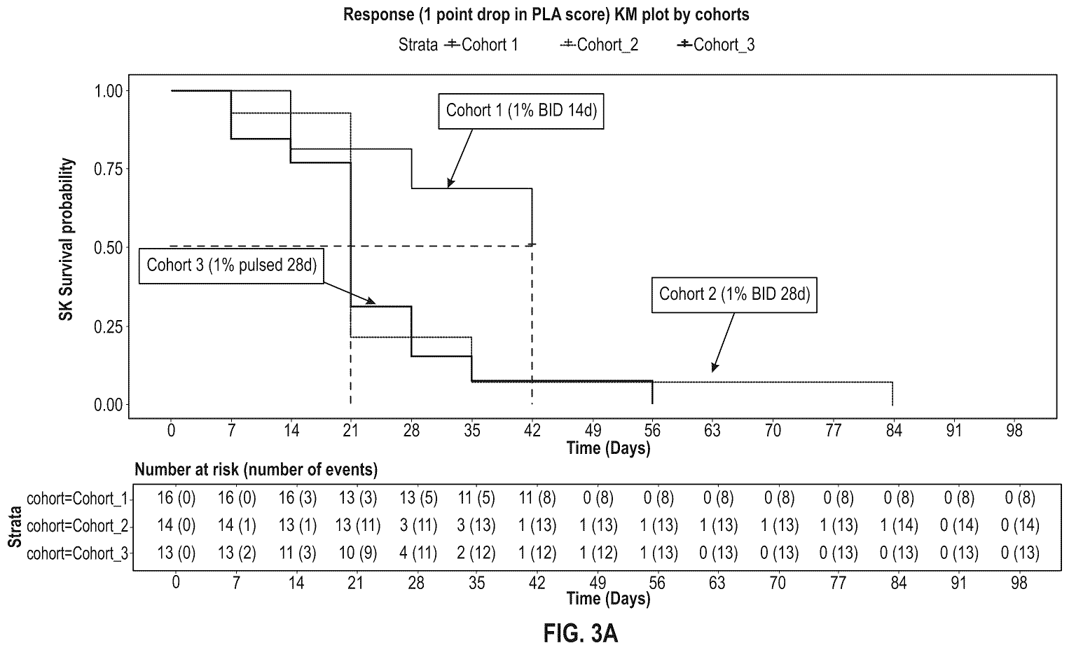

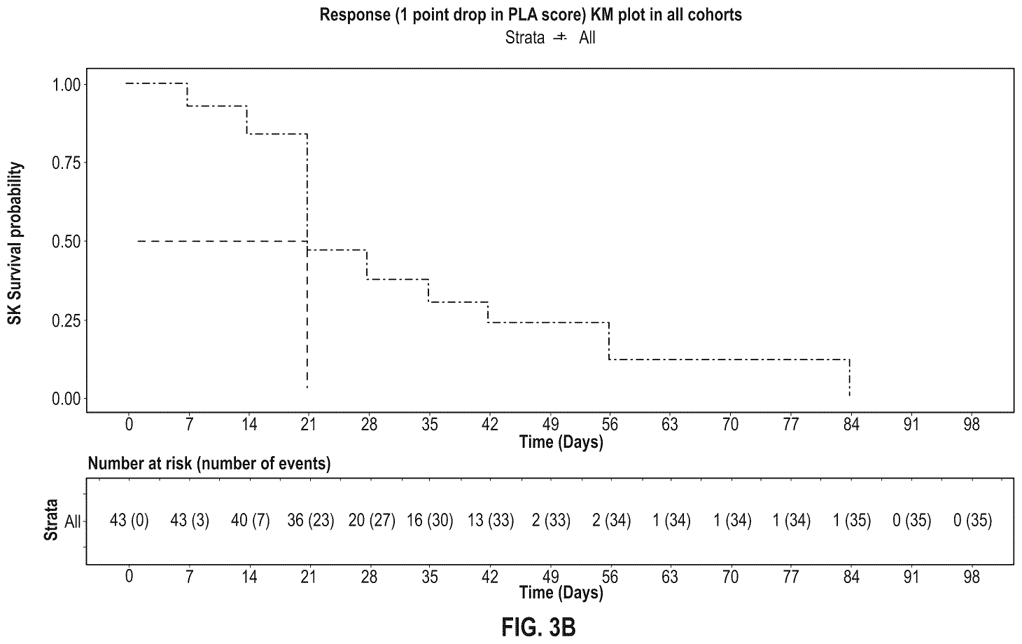

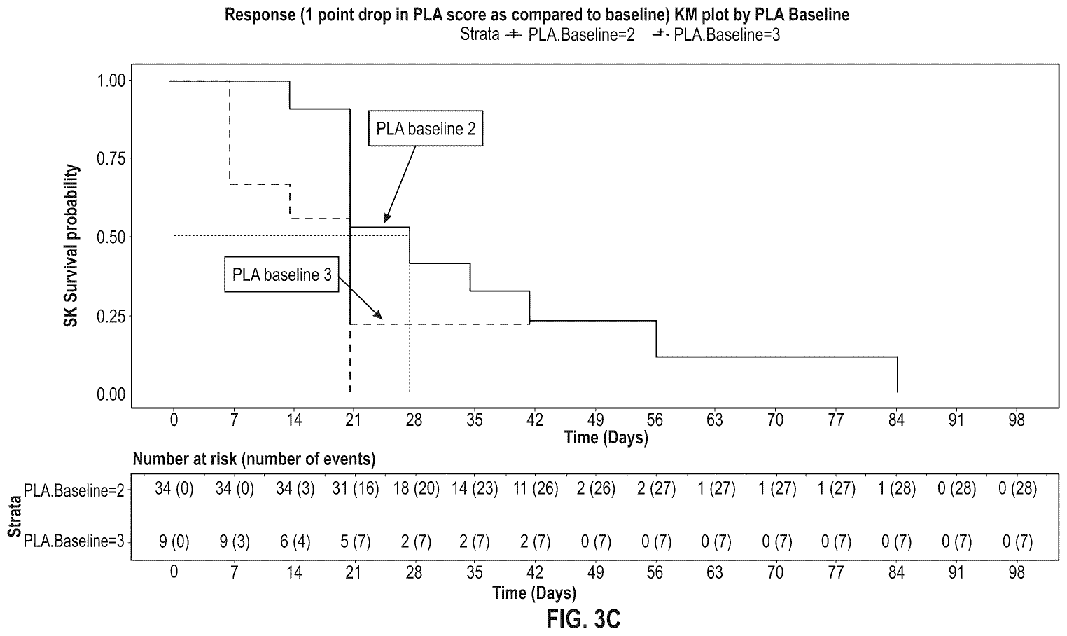

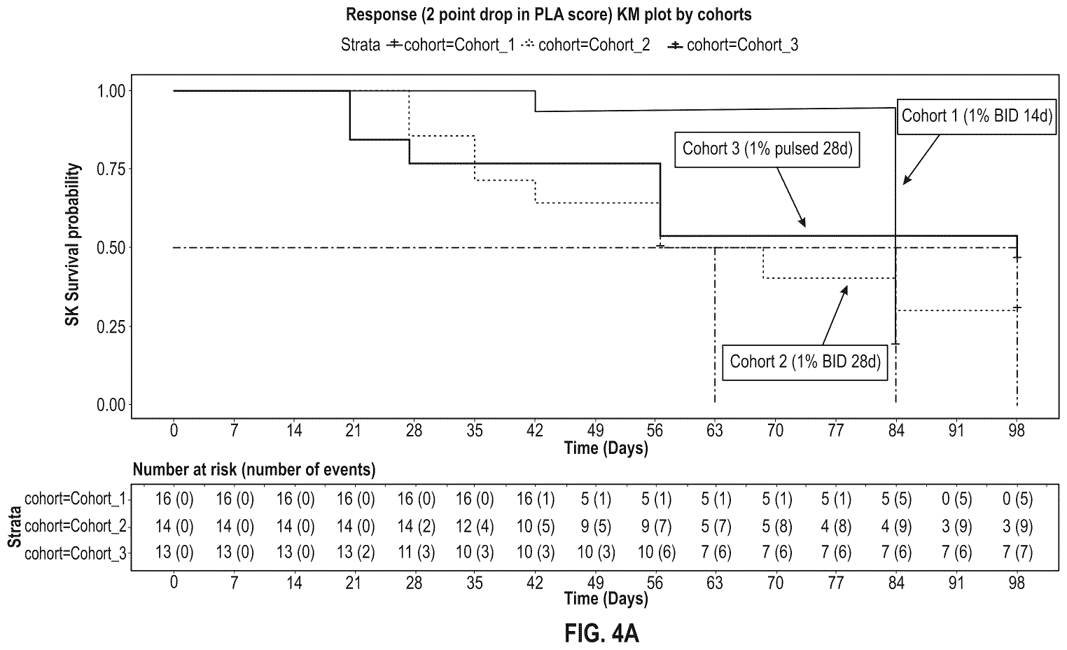

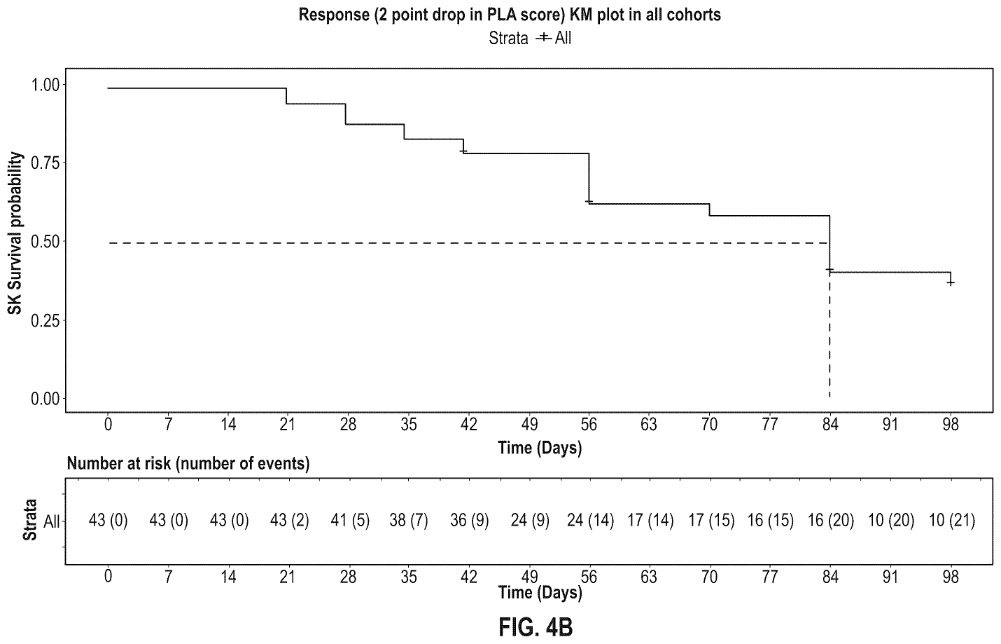

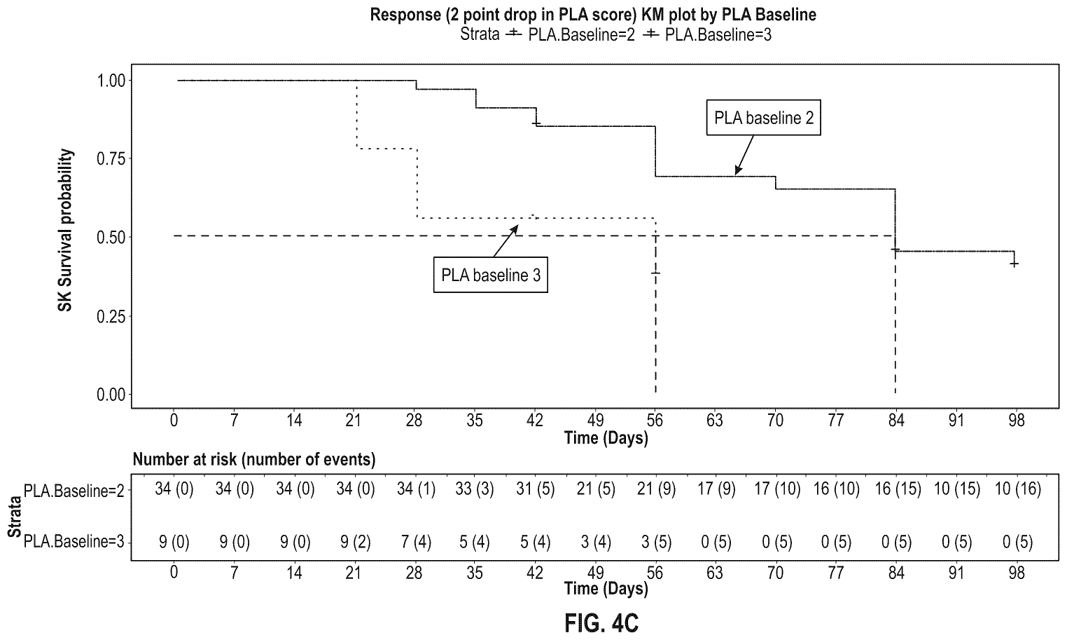

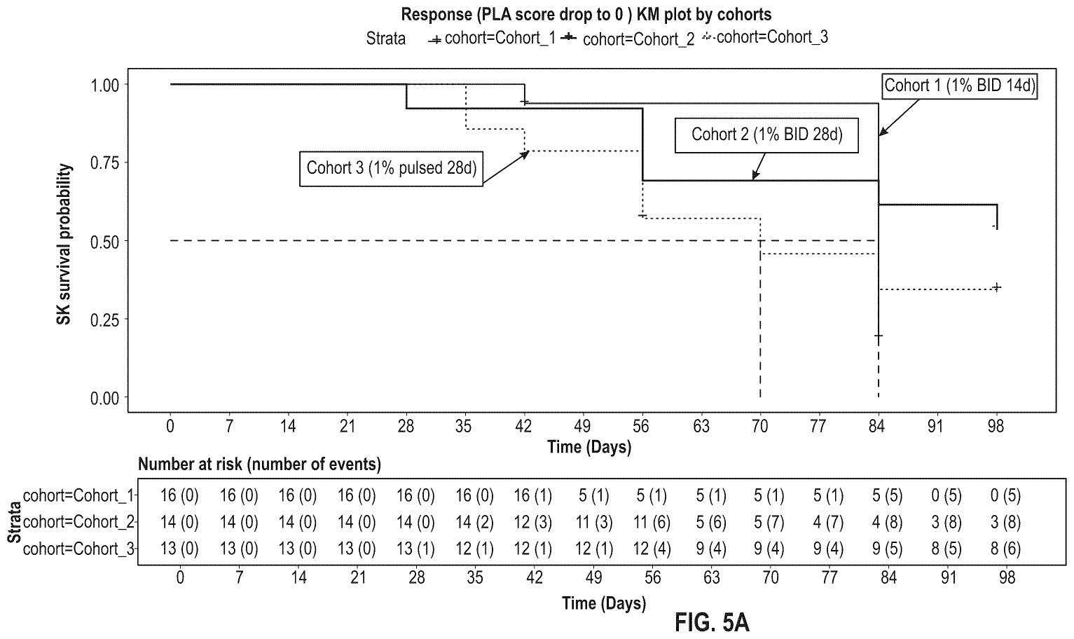

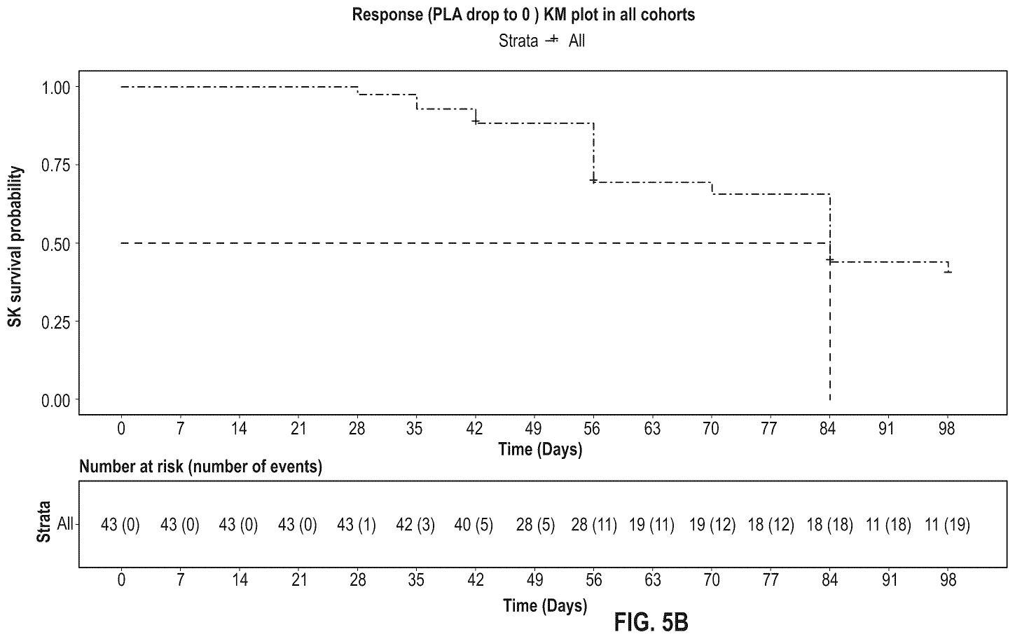

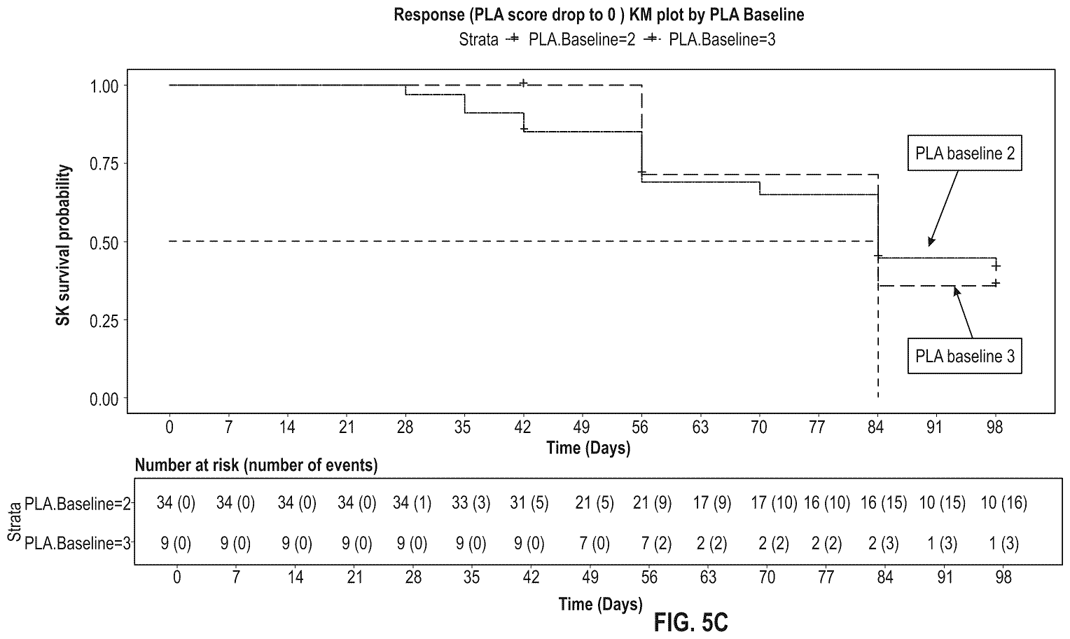

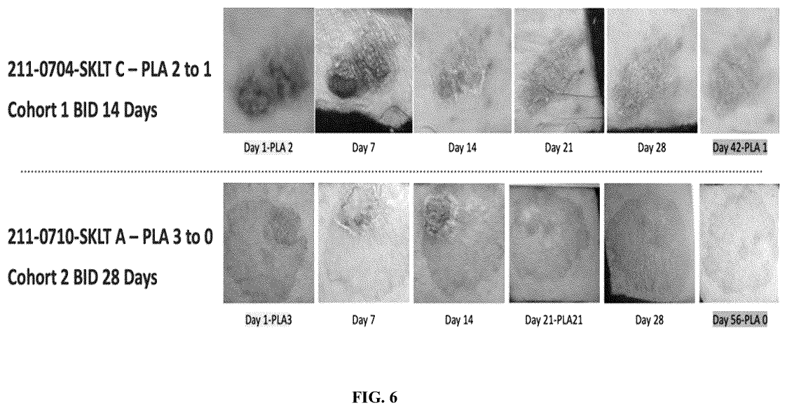

FIGS. 3A-5C provide time-to-event KM plots, as described in the Brief Description of the Drawings. Representative photos demonstrating the response of Composition Ex. 5—Compound 1 to an SK in Cohort 1 (BID for 14 days) and an SK in a background lentigo in Cohort 2 (BID for 28 days), respectively, are shown in FIG. 6. Table 22. In the first three cohorts of the study, there were no SAEs or AEs. Pain, pruritus, erythema, edema and scabbing were rated as none (0), mild (1), moderate (2) or severe (3).

| Visit (Day) | Mild (1) | Moderate (2) | Severe (3) |
|---|---|---|---|
| Baseline | 0 (0.0%) | 0 (0.0%) | 0 (0.0%) |
| 7 | 6 (10.0%) | 0 (0.0%) | 0 (0.0%) |
| 14 | 10 (16.7%) | 2 (3.3%) | 0 (0.0%) |
| 21 | 0 (0.0%) | 0 (0.0%) | 0 (0.0%) |
| 28 | 0 (0.0%) | 0 (0.0%) | 0 (0.0%) |
| 35 | 0 (0.0%) | 0 (0.0%) | 0 (0.0%) |
| 42 | 0 (0.0%) | 0 (0.0%) | 0 (0.0%) |
| 56 | 0 (0.0%) | 0 (0.0%) | 0 (0.0%) |
| 70 | 0 (0.0%) | 0 (0.0%) | 0 (0.0%) |
| 84 | 0 (0.0%) | 0 (0.0%) | 0 (0.0%) |
| 98 | 0 (0.0%) | 0 (0.0%) | 0 (0.0%) |

These data demonstrate that Composition Ex. 5—Compound 1 is an effective, selective and safe treatment for SKs, improving on current treatment methods.

### Example 8: In Human Demonstration of Topical Applications for Treatment of Facial and Occluded Seborrheic Keratosis

### Summary

Promising in vitro and explant studies prompted further investigation to the benefits of Composition Ex. 5—Compound 1 in human clinical studies for the treatment of SKs. This study is a first-in-human open label adaptive design trial to explore the safety and efficacy of Composition Ex. 5—Compound 1 at a concentration of 1.0% or 0.10% with excipient topical gel in patients with Seborrheic Keratosis (SK). A cohort will have a two-week treatment period of twice daily applications followed by a four-week follow-up period. Based on the results at any time from the first and subsequent cohorts, additional cohorts will explore different dosing regimens.

### Methods

The study enrolls up to 35 subjects with 4 SK Target Lesions (SKTLs) each, across seven cohorts with different treatment regimens. The duration of subject participation was approximately 12-16 weeks. Cohorts 4, 5, 6, and 7 were enrolled or planned to be enrolled in the second part of this adaptive design study (results from Cohorts 1, 2, and 3 are described in Example 7). All cohorts had SKs that were treated with Composition Ex. 5—Compound 1.

Cohort 4 (n=5 subjects) each subject receives Composition Ex. 5-1.0% compound 1 application to four Seborrheic Keratosis target lesions (SKTLs) on the face BID (twice a day). Twice daily Composition Ex. 5-1.0% compound 1 is applied to the facial SKTLs for 28 days.

Cohort 5 (n=5 subjects) each subject receives Composition Ex. 5-1.0% compound 1 application to four Seborrheic Keratosis target lesions (SKTLs) on the trunk and extremities under (Tegaderm) occlusion QD (once daily)/TIW (three times a week). The Composition Ex. 5-1.0% compound 1 is applied and kept under occlusion once a day, three times a week.

Cohort 6 (n=5 subjects) each subject receives Composition Ex. 5-1.0% compound 1 application to four Seborrheic Keratosis target lesions (SKTLs) on the Trunk (including intertriginous) or Extremity BID. The Composition Ex. 5-1.0% compound 1 is applied twice daily for 28 days.

Cohort 7 (n=5 subjects) each subject receives Composition Ex. 5-0.1% compound 1 application to four Seborrheic Keratosis target lesions (SKTLs) on the face BID. The Composition Ex. 5-0.10% compound 1 is applied twice daily for 28 days.

All subjects were followed for a minimum of 4 weeks after the last dose. All four SKTL lesions were scored using the PLA scale at each visit.

Primary and secondary efficacy endpoints were based on PLA scoring, a 4 point scale with a 3 indicating a >1 mm thickness SK, a 2 with <1 mm thickness SK, a 1 with nearly clear but residual surface changes suggestive of an SK and 0 with no visible surface changes remaining.

In particular, the primary outcome measure is
1. Proportion of treated SKs with at least a 1 grade improvement in PLA score, change from state of the treatment period through 4 weeks safety follow up [Time Frame: 4 weeks after last dose]

Secondary primary outcome measures include:
2. Proportion of treated SKs with a Physician's Lesion Assessment (PLA) of 0 or 1, Change from start of the treatment period through 4 weeks safety follow-up [Time Frame: 4 weeks after last dose]
3. Proportion of treated SKs with a PLA of 0, Change from start of the treatment period through 4 weeks safety follow-up [Time Frame: 4 weeks after last dose]
4. Proportion of treated SKs with a PLA of 0 or 1, Change from start of the treatment period to week 12 [Time Frame: At week 12]
5. Proportion of treated SKs with a PLA of 0, Change from start of the treatment period to week 12 [Time Frame: At week 12]
6. Proportion of treated SKs with a Subject Self Assessment (SSA) of 0 or 1, Change from start of the treatment period through 4 weeks safety follow-up [Time Frame: 4 weeks after last dose]
7. Proportion of treated SKs with a SSA of 0, Change from start of the treatment period through 4 weeks safety follow-up [Time Frame: 4 weeks after last dose]
8. Proportion of treated SKs with a SSA of 0 or 1, Change from start of the treatment period to week 12 [Time Frame: At week 12]
9. Proportion of treated SKs with a SSA of 0, Change from start of the treatment period to week 12 [Time Frame: At week 12]
10. Time to treated SKs achieving a PLA of 0 or 1, Duration of time from Baseline/Day 1 (PLA 3 or 2) to PLA of 0 or 1 [Time Frame: At study exit. In this adaptive design trial, study exit will be 4 to 10 weeks after completion of the treatment period.]
11. Time to treated SKs achieving a PLA of 0, Duration of time from Baseline/Day 1 (PLA 3 or 2) to PLA of 0 [Time Frame: At study exit. In this adaptive design trial, study exit will be 4 to 10 weeks after completion of the treatment period.]

Inclusion criteria include:
1. At least 18 years of age.
2. Have four eligible SKs on the face, trunk, or extremities. An eligible SK
must: a. Have a clinically typical appearance, b. Have a Physician's Lesion Assessment (PLA) of >2, c. Have a length that is >1 mm and <15 mm, d. Have a width that is >1 mm and <15 mm, e. Have a thickness that is <2 mm, f. Be a discrete lesion, g. Not be covered with hair which, in the Investigator's opinion, would interfere with the study medication treatment or the study evaluations, h. Not be pedunculated
3. Must be in good general health and free of any known disease state or physical condition which, in the Investigator's opinion, might impair evaluation of any target SK lesion or which exposes the subject to an unacceptable risk by study participation.
4. Must be willing and able to follow all study instructions and to attend all study visits.
5. As applicable, technical ability and willingness to apply Investigational Product (IP).
6. Must be able to comprehend and willing to sign an informed consent form (ICF).

Exclusion criteria include:
1. Positive urine pregnancy test, pregnant, lactating, or female of childbearing potential who does not agree to use an active method of birth control for the duration of the study.
2. Have SK lesions that are clinically atypical and/or rapidly growing in size.
3. Presence of multiple eruptive SK lesions (sign of Leser-Trelat)
4. Current systemic malignancy.
5. Any use of the following systemic therapies within the specified period prior to the Screening visit:
a. Retinoids; 180 days, b. Glucocorticosteroids; 28 days, c. Anti-metabolites (e.g., methotrexate); 28 days
6. Any use of the following topical therapies within the specified period prior to the Screening visit on, or in a proximity to any target SK lesion, that in the Investigator's opinion could interfere with the study medication treatment or the study assessments:
a. Laser, light or other energy-based therapy [e.g., intense pulsed, light (IPL), photo-dynamic therapy (PDT)]; 180 days, b. Liquid nitrogen, electrodesiccation, curettage, imiquimod, 5-flurouracil, or ingenol mebutate; 60 days, c. Microdermabrasion or superficial chemical peels; 14 days, d. Glucocorticosteroids or antibiotics; 14 days
7. Occurrence or presence of any of the following within the specified period prior to the Screening visit on or in the proximity of any target SK lesion that, in the Investigator's opinion, could interfere with the study medication treatment or the study assessments:
a. Cutaneous malignancy; 180 days, b. Sunburn; currently, c. Excessive suntan; currently, d. A pre-malignancy (e.g., actinic keratosis); currently, e. Body art (e.g., tattoos, piercing, etc.); currently
8. History of sensitivity to any of the ingredients in the study medications.
9. Any current skin disease (e.g., psoriasis, atopic dermatitis, eczema, sun damage, etc.), or condition (e.g., sunburn, excessive hair, open wounds) that, in the opinion of the Investigator, might put the subject at undue risk by study participation or interfere with the study conduct or evaluations.
10. Participation in an investigational drug trial in which administration of an investigational study medication occurred within 30 days prior to the Screening visit.

TABLE 23
Composition Ex. 5 with 1.0% and 0.1% of compound 1.

| | Compositions (wt/wt %) | |
|---|---|---|
| Components | Ex. 5-1.0% | Ex. 5-Vehicle |
| Compound 1 | 1.06 | — |
| Alcohol (ethanol), 200 Proof, USP | 14.25 | 14.25 |
| Propylene Glycol, USP | 15.00 | 15.00 |
| Sterile Water for Irrigation, USP | 19.64 | 19.64 |
| Transcutol P (2-(2-ethoxyethoxy)ethanol) | 47.00 | 47.00 |
| BHT | 0.05 | 0.05 |
| Phenoxyethanol | 1.00 | 1.00 |
| Klucel MF Pharm (HPC) | 2.00 | 2.00 |
| Total Amount | 100.00 | 100.00 |
| | Compositions (wt/wt %) | |
| Components | Ex. 5-0.1% | Ex. 5-Vehicle |
| Compound 1 | 0.10 | — |
| Alcohol, 200 Proof, USP | 14.25 | 14.25 |
| Propylene Glycol, USP | 15.00 | 15.00 |
| Sterile Water for Irrigation, USP | 20.60 | 19.64 |
| Transcutol P (2-(2-ethoxyethoxy)ethanol) | 47.00 | 47.00 |
| BHT | 0.05 | 0.05 |
| Phenoxyethanol | 1.00 | 1.00 |
| Klucel MF Pharm (HPC) | 2.00 | 2.00 |
| Total Amount | 100.00 | 100.00 |

### Results

The clinical trial is ongoing, and the results reported here are interim results. Cohort 7 is fully enrolled and participants have completed up to the day 28 visit, Cohort 4 is fully enrolled and visits completed to day 14 and some participants up to day 21, Cohort 5 has 4 out 5 potential participants enrolled and participants have completed up to the screening visit and some participants up to day 7, Cohort 6 has not yet been enrolled.

The column in tables 24-29 entitled “Missing” indicates whether there are any participants or lesions of enrolled participants that have not yet completed the visit and have not been assessed on the designated day. The percentage of all anticipated lesions that or individuals who have not yet been scored is also given.

The treatments are well tolerated and an analysis of all SK lesions across all treatments show only mild local tolerability reactions, if any, and no moderate or severe reactions. The highest number of mild tolerability reactions by lesion was 8 or 15.4% of the lesions on day 7 (Table 24) and all other lesions had no reactions. The local tolerability was also assessed on a patient level where each patient had multiple lesions treated. The majority of patients across all treatment groups had no local tolerability reactions and the highest number of mild tolerability reactions was on day 7 when 3 patients or 27.3% of the patients had mild tolerability reactions. There were no moderate or severe reactions (Table 25).

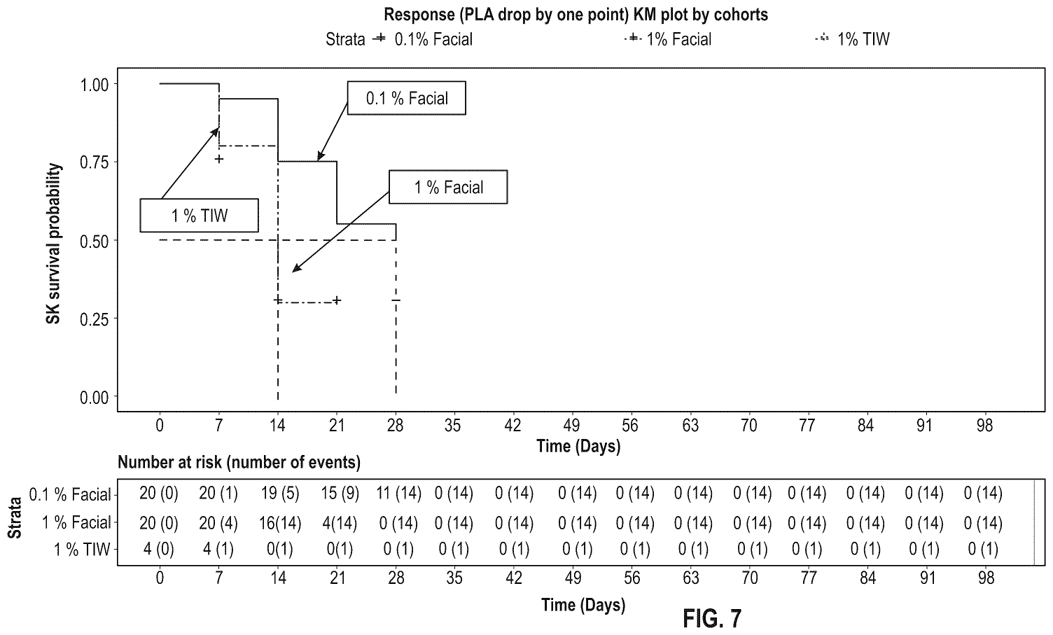

In Cohorts 4 (1% facial) and 7 (0.1% facial), the majority (approximately 70%) of patients saw at least a 1 point decrease in PLA score after 28 days or 21 days, respectively, of treatment (FIG. 7; Table 27 and Table 29). In cohort 5 (1% TIW), at least one patient out of three who had completed the day 7 screening, saw a 1 point decrease in PLA score (Table 28).

TABLE 24
Local Tolerability score for all lesions treated.

| | LTR | LTR | LTR | LTR | |
|---|---|---|---|---|---|
| Visit | none | Mild | Moderate | Severe | |
| (day) | (0) | (1) | (2) | (3) | Missing |
| Screening | 49 (94.2%) | 3 (5.8%) | 0 (0.0%) | 0 (0.0%) | 0 (0.0%) |
| 7 | 36 (69.2%) | 8 (15.4%) | 0 (0.0%) | 0 (0.0%) | 8 (15.4%) |
| 14 | 33 (63.5%) | 7 (13.5%) | 0 (0.0%) | 0 (0.0%) | 12 (23.1%) |
| 21 | 26 (50.0%) | 2 (3.8%) | 0 (0.0%) | 0 (0.0%) | 24 (46.2%) |
| 28 | 20 (38.5%) | 0 (0.0%) | 0 (0.0%) | 0 (0.0%) | 32 (61.5%) |

TABLE 25
Local tolerability score per patient.

| | | | LTR | | |
|---|---|---|---|---|---|
| | LTR | LTR | Moder- | LTR | |
| Visit | none | Mild | ate | Severe | |
| (day) | (0) | (1) | (2) | (3) | Missing |
| Screening | 11 (84.6%) | 2 (15.4%) | 0 (0.0%) | 0 (0.0%) | 0 (0.0%) |
| 7 | 8 (72.7%) | 3 (27.3%) | 0 (0.0%) | 0 (0.0%) | 0 (0.0%) |
| 14 | 7 (70.0%) | 3 (30.0%) | 0 (0.0%) | 0 (0.0%) | 0 (0.0%) |
| 21 | 6 (85.7%) | 1 (14.3%) | 0 (0.0%) | 0 (0.0%) | 0 (0.0%) |
| 28 | 5 (100.0%) | 0 (0.0%) | 0 (0.0%) | 0 (0.0%) | 0 (0.0%) |

TABLE 26
PLA scores for all patients

| | | PLA 1 | | | |
|---|---|---|---|---|---|
| Visit | PLA 0 | (Near | PLA 2 | PLA 3 | |
| (day) | (Clear) | Clear) | (<1 mm) | (>1 mm) | Missing |
| Screening | 0 (0.0%) | 0 (0.0%) | 24 (46.2%) | 28 (53.8%) | 0 (0.0%) |
| 7 | 0 (0.0%) | 5 (9.6%) | 17 (32.7%) | 22 (42.3%) | 8 (15.4%) |
| 14 | 0 (0.0%) | 4 (7.7%) | 30 (57.7%) | 6 (11.5%) | 12 (23.1%) |
| 21 | 0 (0.0%) | 3 (5.8%) | 21 (40.4%) | 4 (7.7%) | 24 (46.2%) |
| 28 | 0 (0.0%) | 11 (21.2%) | 8 (15.4%) | 1 (1.9%) | 32 (61.5%) |

TABLE 27
PLA scores for Cohort 4 (1% facial)

| | | PLA 1 | | | |
|---|---|---|---|---|---|
| Visit | PLA 0 | (Near | PLA 2 | PLA 3 | |
| (day) | (Clear) | Clear) | (<1 mm) | (>1 mm) | Missing |
| Screening | 0 (0.0%) | 0 (0.0%) | 7 (35.0%) | 13 (65.0%) | 0 (0.0%) |
| 7 | 0 (0.0%) | 4 (20.0%) | 4 (20.0%) | 12 (60.0%) | 0 (0.0%) |
| 14 | 0 (0.0%) | 4 (20.0%) | 14 (70.0%) | 2 (10.0%) | 0 (0.0%) |
| 21 | 0 (0.0%) | 0 (0.0%) | 6 (30.0%) | 2 (10.0%) | 12 (60.0%) |
| 28 | 0 (0.0%) | 0 (0.0%) | 0 (0.0%) | 0 (0.0%) | 20 (100.0%) |

TABLE 28
PLA scores for Cohort 5 (1% TIW)

| | | PLA 1 | | | |
|---|---|---|---|---|---|
| | PLA 0 | (Near | PLA 2 | PLA 3 | |
| Visit | (Clear) | Clear) | (<1 mm) | (>1 mm) | Missing |
| Screening | 0 (0.0%) | 0 (0.0%) | 6 (50.0%) | 6 (50.0%) | 0 (0.0%) |
| 7 | 0 (0.0%) | 1 (8.3%) | 1 (8.3%) | 2 (16.7%) | 8 (66.7%) |
| 14 | 0 (0.0%) | 0 (0.0%) | 0 (0.0%) | 0 (0.0%) | 12 (100.0%) |
| 21 | 0 (0.0%) | 0 (0.0%) | 0 (0.0%) | 0 (0.0%) | 12 (100.0%) |
| 28 | 0 (0.0%) | 0 (0.0%) | 0 (0.0%) | 0 (0.0%) | 12 (100.0%) |

TABLE 29
PLA Cohort 7 (0.1% facial)

| Visit | PLA 0 | Near | | | |
|---|---|---|---|---|---|
| (day) | (Clear) | Clear | Thin | Thick | Missing |
| Screen- | 0 (0.0%) | 0 (0.0%) | 11 (55.0%) | 9 (45.0%) | 0 (0.0%) |
| ing | | | | | |
| 7 | 0 (0.0%) | 0 (0.0%) | 12 (60.0%) | 8 (40.0%) | 0 (0.0%) |
| 14 | 0 (0.0%) | 0 (0.0%) | 16 (80.0%) | 4 (20.0%) | 0 (0.0%) |
| 21 | 0 (0.0%) | 3 (15.0%) | 15 (75.0%) | 2 (10.0%) | 0 (0.0%) |
| 28 | 0 (0.0%) | 11 (55.0%) | 8 (40.0%) | 1 (5.0%) | 0 (0.0%) |

### Example 9: Stability of Composition Ex. 5-0.1% and 1.0% Compound 1

The content and purity of Compound 1 in Composition Ex. 5-0.1% and Ex. 5-1.0% following storage up to 6 months are detailed in Table 30.

Percentage recovery (%) compares the response of drug peak to a standard of known concentration in a chromatogram using standard HPLC methods. The change in content (%) of Compound 1 in the compositions following up to 6 months of stability testing was measured starting from t=0 and three technical replicates were performed at each stability time point for each storage condition and each content of Compound 1. There were no deviations from the expected range of content of Compound 1 in Composition Ex. 5-0.1% and 1.0% over time. The acceptable range of Compound 1 content was 90.0%-110.0%

### Apparent pH

The apparent pH of Composition Ex. 5-0.1% Compound 1 and Composition Ex. 5-1.0% Compound 1 following storage up to 6 months at 25° C. and 40° C. is presented in Table 24. Apparent pH was measured using standard equipment and methods in the field.

Apparent pH remained consistent from 0 to 6 months at 25° C. and at 40° C. for Ex. 5-0.1% Compound 1 and Ex. 5-1.0% Compound 1. The pH of Ex. 5-1.0% Compound 1 at both temperatures ranged from 8.49 to 8.62. The pH of Ex. 5-0.1% Compound 1 at both temperatures ranged from 7.78 to 8.18.

### Macroscopic Observations

The macroscopic appearance and macroscopic images against light and dark backgrounds were assessed for Compositions Ex. 5-0.1% Compound 1 and Ex. 5-1.0% Compound 1 following storage up to 6 months at 25° C. and 40° C. (Table 30).

At t=0, all compositions were yellow or slightly yellow, slightly turbid, had medium viscosity and smooth application. The color, viscosity and feel remained consistent over time and temperature. The color in Composition Ex. 5-1.0% Compound 1 was scored as stronger than Composition Ex. 5-0.1% Compound 1.

### Microscopic Observations

The microscopic appearance and microscopic images (non-polarized and polarized light) were assessed using standard microscopy methods for all compositions at the designated time points and storage temperatures.

There was no evidence of drug particulates or crystals in any of the compositions (Table 30).

### Viscosity

The viscosity was assessed for Compositions Ex. 5-0.1% Compound 1 and Ex. 5-1.0% Compound 1 following storage up to 6 months at 25° C. and 40° C. (Table 24). Viscosity was measured using standard methods. The viscosity of Ex. 5-1.0% Compound 1 ranged from 22440 cp to 29600 cp. The viscosity of Composition Ex. 5-0.1% Compound 1 ranged from 12800 cp to 28400 cp.

TABLE 30
Macroscopic, microscopic, apparent pH and average content of Composition Ex.
5 with 1.0% and 0.1% of compound 1. RH = Relative humidity, cP = centipoise.

| | | | | |
|---|---|---|---|---|
| | | | | Average |
| | | | | Compound 1 |
| Macroscopic | Microscopic | Apparent | | Content |
| Appearance | appearance | pH | Viscosity | (n = 3) |

Compound 1 Ex. 5-1.0%

| | | | | | |
|---|---|---|---|---|---|
| T = 0 | pale yellow gel | no crystals | 8.58 | 23800 cp | 98.10% |
| T = 1 mo | pale yellow gel | no crystals | 8.55 | 29600 cp | 97.60% |
| 25° C./60% | | | | | |
| RH | | | | | |
| T = 1 mo | Pale yellow gel | no crystals | 8.57 | 28600 cp | 99.60% |
| 40° C./75% | | | | | |
| RH | | | | | |
| T = 3 mo | light yellow gel | no crystals | 8.62 | 22440 cp | 94.80% |
| 25° C./60% | | | | | |
| RH | | | | | |
| T = 3 mo | Beige gel | no crystals | 8.55 | 23720 cp | 92.6 |
| 40° C./75% | | | | | |
| RH | | | | | |
| T = 6 mo | Pale yellow gel | no crystals | 8.6 | 29200 cp | 104.00% |
| 25° C./60% | | | | | |
| RH | | | | | |
| T = 6 mo | Pale yellow gel | no crystals | 8.49 | 26720 cp | 100.80% |
| 40° C./75% | | | | | |
| RH | | | | | |

Compound 1 Ex. 5-0.1%

| | | | | | |
|---|---|---|---|---|---|
| T = 0 | very slightly | no crystals | 8.05 | 26200 cp | 103.70% |
| | pale yellow gel | | | | |
| T = 1 mo | very slightly | no crystals | 7.94 | 28400 cp | 104% |
| 25° C./60% | pale yellow gel | | | | |
| RH | | | | | |
| T = 1 mo | very pale | no crystals | 7.78 | 27180 cp | 100.60% |
| 40° C./75% | yellow gel | | | | |
| RH | | | | | |
| T = 3 mo | Very light | no crystals | 8.16 | 12800 cp | 102.20% |
| 25° C./60% | yellow gel | | | | |
| RH | | | | | |
| T = 3 mo | very light pale | no crystals | 8.18 | 13920 cp | 98.00% |
| 40° C./75% | yellow gel | | | | |
| RH | | | | | |
| T = 6 mo | very slightly | no crystals | 8.08 | 26800 cp | 103.90% |
| 25° C./60% | pale yellow gel | | | | |
| RH | | | | | |
| T = 6 mo | very slightly | no crystals | 7.86 | 28200 cp | 97.70% |
| 40° C./75% | pale yellow gel | | | | |
| RH | | | | | |

Compound 1 Ex. 5-1.0%

| | | | | | |
|---|---|---|---|---|---|
| T = 0 | pale yellow gel | no crystals | 8.58 | 23800 cp | 98.10% |
| T = 1 mo | pale yellow gel | no crystals | 8.55 | 29600 cp | 97.60% |
| 25° C./60% | | | | | |
| RH | | | | | |
| T = 1 mo | Pale yellow gel | no crystals | 8.57 | 28600 cp | 99.60% |
| 40° C./75% | | | | | |
| RH | | | | | |
| T = 3 mo | light yellow gel | no crystals | 8.62 | 22440 cp | 94.80% |
| 25° C./60% | | | | | |
| RH | | | | | |
| T = 3 mo | Beige gel | no crystals | 8.55 | 23720 cp | 92.6 |
| 40° C./75% | | | | | |
| RH | | | | | |
| T = 6 mo | Pale yellow gel | no crystals | 8.6 | 29200 cp | 104.00% |
| 25° C./60% | | | | | |
| RH | | | | | |
| T = 6 mo | Pale yellow gel | no crystals | 8.49 | 26720 cp | 100.80% |
| 40° C./75% | | | | | |
| RH | | | | | |

Compound 1 Ex. 5-0.1%

| | | | | | |
|---|---|---|---|---|---|
| T = 0 | very slightly | no crystals | 8.05 | 26200 cp | 103.70% |
| | pale yellow gel | | | | |
| T = 1 mo | very slightly | no crystals | 7.94 | 28400 cp | 104% |
| 25° C./60% | pale yellow gel | | | | |
| RH | | | | | |
| T = 1 mo | very pale | no crystals | 7.78 | 27180 cp | 100.60% |
| 40° C./75% | yellow gel | | | | |
| RH | | | | | |
| T = 3 mo | | | | | |
| 25° C./60% | Very light | no crystals | 8.16 | 12800 cp | 102.20% |
| RH | yellow gel | | | | |
| T = 3 mo | very light pale | no crystals | 8.18 | 13920 cp | 98.00% |
| 40° C./75% | yellow gel | | | | |
| RH | | | | | |
| T = 6 mo | very slightly | no crystals | 8.08 | 26800 cp | 103.90% |
| 25° C./60% | pale yellow gel | | | | |
| RH | | | | | |
| T = 6 mo | very slightly | no crystals | 7.86 | 28200 cp | 97.70% |
| 40° C./75% | pale yellow gel | | | | |
| RH | | | | | |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating a cutaneous lesion, the method comprising topically administering to the cutaneous lesion a composition comprising:
(i) a compound having the structure: 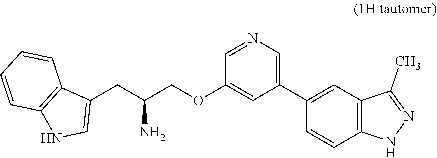 [SMILES: Cc1n[nH]c2ccc(-c3cncc(OC[C@@H](N)Cc4c[nH]c5ccccc45)c3)cc12]
or a salt thereof, and/or
(ii) a compound having the structure: 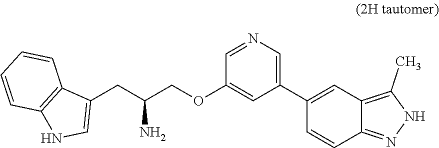 [SMILES: Cc1[nH]nc2ccc(-c3cncc(OC[C@@H](N)Cc4c[nH]c5ccccc45)c3)cc12]
or a salt thereof,
wherein the total amount of (i), (ii), or (i) and (ii) present in the composition is 0.01% to 10%, 0.01% to 5%, or 0.1% to 1% by weight.

2. The method of claim 1, wherein the cutaneous lesion is a keratosis.

3. The method of claim 2, wherein the keratosis is a seborrheic keratosis.

4. The method of claim 1, wherein the composition is administered one, two, three, or four times a day.

5. The method of claim 4, wherein the composition is administered two times a day for about 14 days to about 28 days, or for about 2 weeks to about 8 weeks.

6. (canceled)

7. The method of claim 1, wherein the composition is administered in a cycle comprising twice daily administration for about 4 days, followed by no administration for about 4 days.

8. (canceled)

9. (canceled)

10. (canceled)

11. (canceled)

12. (canceled)

13. The method of claim 1, further comprising occlusion of the cutaneous lesion.

14. The method of claim 1, wherein the cutaneous lesion is present on a human subject.

15. (canceled)

16. The method of claim 1, wherein treating comprises an improvement of one grade or more in Physician's Lesion Assessment (PLA) score as compared to before administering the composition.

17. (canceled)

18. (canceled)

19. (canceled)

20. (canceled)

21. The method of claim 1, wherein treating comprises inducing apoptosis of a keratinocyte in the cutaneous lesion.

22. (canceled)

23. The method of claim 1, wherein the composition is a gel formulation.

24. (canceled)

25. A composition comprising:
(i) a compound having the structure: 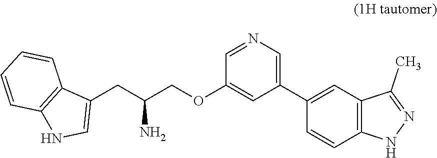 [SMILES: Cc1n[nH]c2ccc(-c3cncc(OC[C@@H](N)Cc4c[nH]c5ccccc45)c3)cc12]
or a salt thereof, and/or
(ii) a compound having the structure: 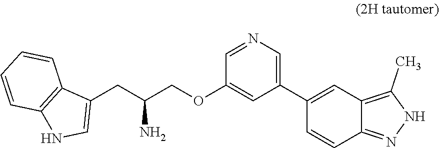 [SMILES: Cc1[nH]nc2ccc(-c3cncc(OC[C@@H](N)Cc4c[nH]c5ccccc45)c3)cc12]
or a salt thereof,
wherein the total amount of (i), (ii), or (i) and (ii) present in the composition is 0.01% to 10%, 0.01% to 5%, or 0.1% to 1% by weight; and wherein the composition further comprises ethanol, propylene glycol, 2-(2-ethoxyethoxy)ethanol, and hydroxypropyl cellulose.

26. The composition of claim 25, wherein the composition is a gel formulation.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The composition of claim 25, wherein the propylene glycol is present in an amount of 10% to 20% by weight of the composition.

40. The composition of claim 25, wherein the 2-(2-ethoxyethoxy)ethanol is present in an amount of 40% to 60% by weight of the composition.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The composition of claim 25, wherein the hydroxypropyl cellulose is present in an amount of from 0.5% to 5% by weight of the composition.

56. (canceled)

57. The composition of claim 25, wherein ethanol is present in an amount of 10% to 20% by weight; propylene glycol is present in an amount of from 10% to 20% by weight; 2-(2-ethoxyethoxy)ethanol is present in an amount of 40% to 60% by weight; and hydroxypropyl cellulose is present in an amount of 1% to 3% by weight of the composition.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. The composition of claim 25, wherein the composition is a topical composition.

73. (canceled)

74. (canceled)

75. A method of treating a skin disease, condition or disorder in a subject in need thereof, comprising administering to the subject a composition of claim 25.

76. (canceled)

77. The method of claim 75, wherein the skin disease, condition or disorder is seborrheic keratosis.

78. (canceled)

79. (canceled)

80. (canceled)

81. (canceled)

82. (canceled)

83. (canceled)

84. (canceled)

85. (canceled)

86. (canceled)

87. (canceled)
